SEQUENCE LISTING (38 sequences)

SEQ ID NO: 1 - DNA, 5193 bp, Artificial Sequence
  plasmid-pCP4-N
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaccggtggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgattaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 2 - DNA, 5338 bp, Artificial Sequence
  plasmid-pCP4-N1
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggaggcaggtggacaggaaggttctaatgttcttaaggcacaggaactgggacatctgggcccggaaagcctttttctctgtgatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgattaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 3 - DNA, 5348 bp, Artificial Sequence
  plasmid-pCP4-N2
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggtaccaagcttcgaggcaggtggacagcttggttctaatgaagttaaccctgtcgttctgcgacatctgggcccggaaagcgtttaactgatggatggaacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgattaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 4 - DNA, 5324 bp, Artificial Sequence
  plasmid-pCP4-N3
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaagggcgaattcggatccgcggccgccttaagctcgaggcaggtggacaggaaggttctaatgttctatagggtctgcttgtcgctcatctgggcccggagatgcgtaaagtcagacatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgattaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 5 - DNA, 4980 bp, Artificial Sequence
  plasmid-pCP5-P
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaccggtggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacttaagagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 6 - DNA, 5123 bp, Artificial Sequence
  plasmid-pCP5-P1
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggaggcaggtggacaggaaggttctaatgttcttaaggcacaggaactgggacatctgggcccggaaagcctttttctctgtgatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacttaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 7 - DNA, 5133 bp, Artificial Sequence
  plasmid-pCP5-P2
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggtaccaagcttcgaggcaggtggacagcttggttctaatgaagttaaccctgtcgttctgcgacatctgggcccggaaagcgtttaactgatggatggaacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacttaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 8 - DNA, 5109 bp, Artificial Sequence
  plasmid-pCP5-P3
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaagggcgaattcggatccgcggccgccttaagctcgaggcaggtggacaggaaggttctaatgttctatagggtctgcttgtcgctcatctgggcccggagatgcgtaaagtcagacatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacttaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 9 - DNA, 5400 bp, Artificial Sequence
  plasmid-pCP4-H
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaccggtggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 10 - DNA, 5545 bp, Artificial Sequence
  plasmid-pCP4-H1
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggaggcaggtggacaggaaggttctaatgttcttaaggcacaggaactgggacatctgggcccggaaagcctttttctctgtgatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 11 - DNA, 5555 bp, Artificial Sequence
  plasmid-pCP4-H2
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggtaccaagcttcgaggcaggtggacagcttggttctaatgaagttaaccctgtcgttctgcgacatctgggcccggaaagcgtttaactgatggatggaacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 12 - DNA, 5531 bp, Artificial Sequence
  plasmid-pCP4-H3
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaagggcgaattcggatccgcggccgccttaagctcgaggcaggtggacaggaaggttctaatgttctatagggtctgcttgtcgctcatctgggcccggagatgcgtaaagtcagacatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 13 - DNA, 4749 bp, Artificial Sequence
  plasmid-pCP4-Z
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaccggtggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 14 - DNA, 4894 bp, Artificial Sequence
  plasmid-pCP4-Z1
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggaggcaggtggacaggaaggttctaatgttcttaaggcacaggaactgggacatctgggcccggaaagcctttttctctgtgatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 15 - DNA, 4904 bp, Artificial Sequence
  plasmid-pCP4-Z2
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggtaccaagcttcgaggcaggtggacagcttggttctaatgaagttaaccctgtcgttctgcgacatctgggcccggaaagcgtttaactgatggatggaacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 16 - DNA, 4880 bp, Artificial Sequence
  plasmid-pCP4-Z3
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaagggcgaattcggatccgcggccgccttaagctcgaggcaggtggacaggaaggttctaatgttctatagggtctgcttgtcgctcatctgggcccggagatgcgtaaagtcagacatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgatgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 17 - DNA, 4803 bp, Artificial Sequence
  plasmid-pCP5-B
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaccggtggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacttaagagacaggatgaggatcgtttcgcaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 18 - DNA, 4948 bp, Artificial Sequence
  plasmid-pCP5-B1
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggaggcaggtggacaggaaggttctaatgttcttaaggcacaggaactgggacatctgggcccggaaagcctttttctctgtgatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacttaagagacaggatgaggatcgtttcgcaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 19 - DNA, 4958 bp, Artificial Sequence
  plasmid-pCP5-B2
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccagggcgaattccagcacactggcggccgttactagtggatccgagctcggtaccaagcttcgaggcaggtggacagcttggttctaatgaagttaaccctgtcgttctgcgacatctgggcccggaaagcgtttaactgatggatggaacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacttaagagacaggatgaggatcgtttcgcaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 20 - DNA, 4934 bp, Artificial Sequence
  plasmid-pCP5-B3
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctcgttaattaaaagcttctcgagagtttaaacaggcgcgccaagggcgaattcggatccgcggccgccttaagctcgaggcaggtggacaggaaggttctaatgttctatagggtctgcttgtcgctcatctgggcccggagatgcgtaaagtcagacatccggtacagtccttctgcggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggtacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaatatcgatatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacttaagagacaggatgaggatcgtttcgcaagagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 21 - DNA, 34 bp, artificial sequence
  signaling probe to Chromo-Tag A
gccagtcccagttcctgtgccttaagaacctcgc

SEQ ID NO: 22 - DNA, 34 bp, artificial sequence
  signaling probe to Chromo-Tag B
gcgagtcgcagaacgacagggttaacttcctcgc

SEQ ID NO: 23 - DNA, 34 bp, artificial sequence
  signaling probe to Chromo-Tag C
gcgagagcgacaagcagaccctatagaacctcgc

SEQ ID NO: 24 - DNA, 34 bp, artificial sequence
  tas1r2-a
gcgaggagacggatgaggtgaaatagaagctcgc

SEQ ID NO: 25 - DNA, 34 bp, artificial sequence
  tas1r2-b
gcgagattgtgaagcagaggggaaagaggctcgc

SEQ ID NO: 26 - DNA, 34 bp, artificial sequence
  tas1r2-c
gcgagaccttcacttcatactccttgcacctcgc

SEQ ID NO: 27 - DNA, 34 bp, artificial sequence
  tas1r3-a
gcgaggtttcccctgatttcctgtgttccgtcgc

SEQ ID NO: 28 - DNA, 34 bp, artificial sequence
  tas1r3-b
gcgagcacccacagcttcaggtcgtactcctcgc

SEQ ID NO: 29 - DNA, 34 bp, artificial sequence
  tas1r3-c
gcgaggaccactcatcctggccacaaaagctcgc

SEQ ID NO: 30 - DNA, 99 bp, artificial sequence
  Chromo-Tag A
gcaggtggacaggaaggttctaatgttcttaaggcacaggaactgggacatctgggcccggaaagcctttttctctgtgatccggtacagtccttctgc

SEQ ID NO: 31 - DNA, 97 bp, artificial sequence
  Chromo-Tag B
gcaggtggacagcttggttctaatgaagttaaccctgtcgttctgcgacatctgggcccggaaagcgtttaactgatggatggaacagtccttctgc

SEQ ID NO: 32 - DNA, 99 bp, artificial sequence
  Chromo-Tag C
gcaggtggacaggaaggttctaatgttctatagggtctgcttgtcgctcatctgggcccggagatgcgtaaagtcagacatccggtacagtccttctgc

SEQ ID NO: 33 - DNA, 52 bp, artificial sequence
  Spacer 1
agggcgaattccagcacactggcggccgttactagtggatccgagctcggag

SEQ ID NO: 34 - DNA, 64 bp, artificial sequence
  Spacer 2
agggcgaattccagcacactggcggccgttactagtggatccgagctcggtaccaagcttcgag

SEQ ID NO: 35 - DNA, 38 bp, artificial sequence
  Spacer 3
aagggcgaattcggatccgcggccgccttaagctcgag

SEQ ID NO: 36 - RNA, 99 nt, Artificial Sequence
  Synthetic Construct
gcagguggacaggaagguucuaauguucuuaaggcacaggaacugggacaucugggcccggaaagccuuuuucucugugauccgguacaguccuucugc

SEQ ID NO: 37 - RNA, 97 nt, Artificial Sequence
  Synthetic Construct
gcagguggacagcuugguucuaaugaaguuaacccugucguucugcgacaucugggcccggaaagcguuuaacugauggauggaacaguccuucugc

SEQ ID NO: 38 - RNA, 99 nt, Artificial Sequence
  Synthetic Construct
gcagguggacaggaagguucuaauguucuauagggucugcuugucgcucaucugggcccggagaugcguaaagucagacauccgguacaguccuucugc

(19) United States
(12) Patent Application Publication
(10) Pub. No.: US 2024/0117417 A1
(43) Pub. Date: Apr. 11, 2024

(54) CELL ENGINEERING COMPOSITIONS AND METHODS USING FLUOROGENIC OLIGONUCLEOTIDE SIGNALING PROBES AND FLOW CYTOMETRY

(71) Applicants: Secondcell Bio, LLC, Brooklyn, NY (US); Chromocell Corporation, North Brunswick, NJ (US)

(72) Inventors: Dennis James SAWCHUK, Fanwood, NJ (US); Olga Anatoliyevna DEDOVA, East Brunswick, NJ (US); Kambiz SHEKDAR, Brooklyn, NY (US)

(21) Appl. No.: 18/261,606

(22) PCT Filed: Jan. 14, 2022
(86) PCT No.: PCT/US2022/012533

§ 371 Date: Jul. 14, 2023

Related U.S. Application Data
(60) Provisional application No. 63/138,257, filed on Jan. 15, 2021.

(51) Int. Cl. C12Q 1/6841; C12N 15/85; G01N 15/01; G01N 15/1434

(52) U.S. Cl. CPC C12Q 1/6841; C12N 15/85; G01N 15/01; G01N 15/1434; G01N 2015/1006

(57) ABSTRACT

The present disclosure relates to novel expression vectors, host cells and methods useful to produce cells comprising one or more sequences of interest.

### CROSS-REFERENCE TO RELATED APPLICATIONS

This application claims priority to U.S. provisional application 63/138,257, filed Jan. 15, 2021, the contents of which are hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 217352000140SEQLIST.TXT, date recorded: Jan. 12, 2022, size: 136,524 bytes).

### FIELD

Chromovert® Technology is disclosed as a cell engineering technology to detect and purify living cells based on expression of one or more sequences of interest. Novel compositions and methods to practice the invention are disclosed.

### BACKGROUND

Despite multiple advances in cell engineering including Clustered Regularly Interspaced Short Palindromic Repeats (CRISPR®), the engineering of cells to comprise one or more added sequences of interest or genes of interest remains challenging (Nowogrodzki 2019). Chromovert® Technology relies on fluorogenic oligonucleotide signaling probes originally reported for in tubo qRT-PCR applications (Marras 1999) transfected into living cells for use to detect and isolate cells that express desired genetic sequences. Briefly, each terminus of the signaling probe is covalently linked to a fluorophore or quencher paired to absorb its emission, for instance via fluorescence-resonance energy transfer (FRET). The termini are designed to form a 4 to 7 base-pair stem juxtaposing the fluorophore and quencher pair. In the presence of target sequence, hybridization of the sequence-specific probe results in a fluorogenic conformational change. Flow cytometry is then used to detect and isolate positive cells that fluoresce above background. Thousands of individual clones comprising and/or expressing one or more sequences of interest are isolated and expanded using manual or automated cell culture methods. Functional testing over time in the absence of selective pressure is used to select final clones.

### SUMMARY

Provided herein is an expression vector comprising a sequence of interest, the vector comprising: in the following operative order, a promoter, a multiple cloning site, a spacer, and a detection tag. In some embodiments, the detection tag is an untranslated sequence that is transcribed.

In some embodiments, the spacer sequence comprises from about 4 to about 630 nucleotides. In some embodiments, the spacer sequence comprises the nucleic acid sequence of SEQ ID NO: 33, 34, or 35.

In some embodiments, the detection tag comprises the nucleotide sequence set forth in SEQ ID NOs: 30, 31, or 32.

In some embodiments, the promoter is selected from the group consisting of a CMV promoter, a TK promoter, a SV40 promoter and an EF-la promoter. In some embodiments, the promoter is a CMV promoter.

In some embodiments, the expression vector further comprises a first antibiotic selection marker and a second antibiotic selection marker. In some embodiments, the first antibiotic selection marker is a bacterial selection marker. In some embodiments, the second antibiotic selection marker is a mammalian selection marker.

In some embodiments, the expression vector comprises the nucleotide sequence set forth in SEQ ID NO: 2, 3, 4, 6, 7, 8, 10, 11, 12, 14, 15, 16, 18, 19, or 20.

In some embodiments, the expression vector further comprises a sequence of interest. In some embodiments, the sequence of interest is located between the promoter and the spacer. In some embodiments, the sequence of interest comprises a gene, a cDNA, or an RNA. In some embodiments, the sequence of interest comprises a mutated, spliced, or processed form of a gene, cDNA, or an RNA. In some embodiments, the sequence of interest is selected from the group consisting of a GPCR, ion channel and ion channel subunit. In some embodiments, the ion channel and ion channel subunit is selected from GABAA, CFTR, ENAC and NaV. In some embodiments, the sequence interest comprises a human sensory gene, a taste gene, or an odorant receptor gene. In some embodiments, the human sensory gene, taste gene, or odorant receptor gene is selected form the group consisting of bitter, salt, sour, umami, hot, pepper, cool, mint, fat, fatty acid, kokumi, mouth feel, touch, touch sensation, mechano sensation, pressure sensing and tingle receptor genes. In some embodiments, the sequence of interest comprises a gene from a DNA virus, a gene from an RNA virus, a gene from a retrovirus, a gene from an adeno-associated virus, a gene from an adenovirus, a gene from a lentivirus, a gene from a herpes virus, an HIV gene, or a gene from a coronavirus. In some embodiments, the sequence of interest comprises a gene from SARS, a gene from MERS, or a gene from SARS-CoV-2 virus. In some embodiments, the sequence of interest is selected from a viral gene used to produce viral particles for use in gene or cell therapy. In some embodiments, the sequence of interest is selected from the group consisting of a gene encoding a cellular protein, a gene encoding a membrane protein, a gene encoding a cytosolic protein, a gene encoding a nuclear protein, a gene encoding a multi-subunit protein, and a gene encoding a secreted protein. In some embodiments, the sequence of interest comprises a drug target or a gene encoding a biologic. In some embodiments, the sequence of interest is selected from a gene, an mRNA, or a cDNA involved in unfolded protein response, cell viability, protein production, protein folding, protein assembly, protein modification, glycosylation, proteolysis, secretion, cell membrane integration, or cell surface presentation. In some embodiments, the sequence of interest is selected from the group consisting of full length and spliced ATF, ATF6, ATF6-Alpha, ATF6A, ATF6B, IRE1 alpha, IRE1α, IRE1 beta, IRE1β, PERCDC, ATF4, YY1, NF-YA, NF-YB, NF-YC, XBP1, XBP2, EDEM1, EDEM2, NRF2, HERP, XIAP, GADD34, PPIA, PPIB, PPIG, DNAJC3, DNAJC6, PRDM1, BLIMP-1, CRT, CALR, CNX, PDIA3, ERp57, HSPA5, BiP, SIL1, BAP, DNAJB11, ERdj3, CaBP1, HSP90B1, GRP-94, PDIA4, ERp72, cyclophilin B, SDF2, SDF2L1, ERO1A, ERO1B, ERAD pathway gene, MAN1B1, mannosidase 1, SYN1, HRD1, STC1, STC2, SERCA1, SERCA2, RPGR, COD1, ISYNA1, INO1, SREBP1, SREBP2, SREBP1DC, SREBP2DC, PC, PYC, Sec61A1, Sec61A2, Sec61B, Sec61G, BCL2, BCL2L1, Bcl-2sp, Bcl-XL, BCL2L11, Bim, XRCC6, Ku70, VDAC2, BCAP31, BAP31, YWHAE, and 14-3-3.

Also provided herein is a method for isolating a cell expressing a gene of interest comprising (a) providing a mixed population of cells at a concentration from about 0.125 to about 20×10⁶ cells/mL in a medium wherein at least a portion of the mixed population of cells expresses the gene of interest; (b) exposing the mixed population of cells to about 0.01625 nM to 27.778 uM of a fluorescent signaling probe; (c) detecting fluoresce of cells within the mixed population of cells; and (d) isolating a cell that fluoresces above background, wherein fluorescence above background indicates that the cell expresses the gene of interest. In some embodiments, the mixed population of cells is exposed to the fluorescent signaling probe is provided at a concentration from about 0.25 uM to about 50 uM in a second medium in step (b). In some embodiments, the signaling probe hybridizes with to the gene of interest. In some embodiments, the fluorescent signaling probe further comprises a quencher. In some embodiments, the cell expresses a second gene of interest.

In some embodiments, step (b) further comprises exposing the mixed population of cells to a second fluorescent signaling probe. In some embodiments, the first and second fluorescent signaling probes comprise different fluorophores. In some embodiments, step (b) is performed using a transfection reagent.

In some embodiments, the transfection reagent is Lipofectamine or jetPEI. In some embodiments, the transfection agent is provided at a concentration from about 1.875 pM to 13.636 uM.

In some embodiments, step (b) is performed using electroporation. In some embodiments, the medium in step (a) is a serum-free medium. In some embodiments, the second medium in step (b) is a serum-free medium. In some embodiments, the media in step (a) and step (b) are selected from the group consisting of Dulbecco's Modified Eagle Medium (DMEM), Opi-MEM Reduced Serum Media and Ham's F12 Nutrient Mixture media.

In some embodiments, the method further comprises isolating a cell that fluoresces above background using flow cytometry.

In some embodiments, the cell is a eukaryotic or prokaryotic cell. In some embodiments, the cell is a mammalian cell. In some embodiments, the mammalian cell is selected from NS0, CHO, COS, Perc6, HEK-293, HEK 293T, HUVECs, 3T3 and HeLa cells. In some embodiments, the mammalian cell is selected from CHO, HEK-293 and HEK 293T cells. In some embodiments, the cell is a yeast, insect, fungus, or plant, human, primate, bovine, porcine, feline, marsupial, or murine cell. In some embodiments, the cell is selected from primary, transformed, oncogenically transformed, virally transformed, immortalized, conditionally transformed, explants, or a cell from a tissue section. In some embodiments, the cell is selected from the group consisting of Chinese hamster ovary (CHO) cells, human embryonic kidney (HEK) cells, cells of a cell line, established neuronal cell lines, pheochrornocytomas, neuroblastomas fibroblasts, rhabdomyosarcomas, dorsal root ganglion cells, NS0 cells, CV-1 (ATCC CCL 70), COS-1 (ATCC CRL 1650), COS-7 (ATCC CRL 1651), CHO/dhFr- (ATCC CCR-9096), CHO 1-15 (ATCC CRL 9609), 16.4 CHO (ATCC CRL-12023) CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), L-cells, HEK-293 (ATCC CRL1573), HEK-293 (ECACC 85120602), HEK-293T (ECACC 12022001), 293-Hektor (ECACC 05030204), 293 N3S (ECACC 92052131), 293 GTP-AC-free (ECACC 05011003), 293TGPRT+R1 (ECACC 04072121), 293TGPRT+R1-A (ECACC 04072120), PC12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), CHO-K1 (ECACC 85051005), Vero (ATCC CCL 81), Caco-2 (ATCC HTB 37.), K562 (ATCC CCL 243), Jurkat (ATCC TB-152), Per.C6, Huvec (ATCC Human Primary PCS 100-010, Mouse CRL 2514, CRL 2515, CRL 2516), HuH-7D12 (ECACC 01042712), 293 (ATCC CRL 10852), A549 (ATCC CCL 185), IMR-90 (ATCC CCL 186), MCF-7 (ATC HTB-22), U-2 OS (ATCC HTB-96), and T84 (ATCC CCL 248), any established cell line (polarized or nonpolarized), any ATCC and any ECACCcell line.

Also provided herein is a method for preparing a cell comprising a sequence of interest, the method comprising: (a) incubating the expression vector of any one of claims 1-26 comprising the sequence of interest with a cell; (b)selecting a cell comprising an integrated expression vector. In some embodiments, antibiotic resistance is used to select cells in step (b).

Also provided herein is a cell comprising any one of the expression vectors disclosed herein.

This application further provides a cell prepared by the methods disclosed herein. In some embodiments, the cell is a mammalian cell. In some embodiments, the mammalian cell is selected from the group consisting of HEK293T and CHO cells.

Also provided herein is a composition comprising the cell disclosed herein.

In some embodiments, provided herein is a kit comprising an expression vector disclosed herein. In some embodiments, the kit further comprises a cell. In some embodiments, the cell is selected from the group consisting of a NS0, CHO, COS, Perc6, HEK-293, HEK 293T, HUVECs, 3T3 and a HeLa cell. In some embodiments, the kit further comprises a signaling probe. In some embodiments, the signaling probe comprises a fluorophore and a quencher. In some embodiments, the signaling probe comprises the nucleotide sequence set forth in SEQ ID NO: 21, 22 or 23. In some embodiments, the kit comprises a transfection agent. In some embodiments, the transfection agent is selected from Lipofectamine and jetPEI®.

All references disclosed herein are hereby incorporated by reference in their entireties, including the following references.
Babich O, Garyantes T, Luo R, Palling D, Venkatachalan S, Wang-Fischer Y (2016) Sodium channel modulators for the treatment of pain and diabetes, U.S. Pat. No. 9,458,118 Fornius M (2013) Treatment of pulmonary edema by ENaC activators/stimulators, Curr Mol Pharmacol 6:13-27
Marras S A, Kramer F R and Tyagi S (1999) Multiplex detection of single-nucleotide variations using molecular beacons, Genet Anal 14:151-6
Matalon S, Bartoszewski R, Collawn J F (2015) Role of epithelial sodium channels in the regulation of lung fluid homeostasis, Am J Physiol Lung Cell Mol Physiol 309:L1229-38
Nowogrodzki A (2019) The challenge of using CRISPR to knock in genes, The Scientist
Obergrussberger A et al (2014) Automated patch clamp analysis of nAChα7 and Na(V)1.7 channels, Curr Protoc Pharmacol 65:11.13.1-48
O'Malley H A, Isom L L (2015) Sodium channel R subunits: emerging targets in channelopathies, Annu Rev Physiol 77:481-504
Olsen R W, Sieghart W (2008) International Union of Pharmacology LXX subtypes of γ Aminobutyric Acid A receptors: classification on the basis of subunit composition, pharmacology, and function, Update Pharmacol Rev Vol. 60:243-60
Roper S D (2015) The taste of table salt, Pflugers Arch Vol. 467:457-463
Shekdar K and Langer J (2017) Cell lines expressing ENaC and methods of using them, U.S. Pat. No. 9,534,035
Shekdar K and Langer J (2011) Cell lines expressing GABA receptor and methods of using them, US Pat. Publ. No. 2011/0003711
Shekdar K, Sawchuk D, Shah P (2012) Novel cell lines and methods, US Pat. Publ. No. 2012/0015841
Shekdar K, Venkatachalan S, Langer J and Sawchuk D (2012) Cell lines expressing CFTR and methods of using them, US Pat. Publ. No. 2012/0058918
Zuker M (2003) Mfold web server for nucleic acid folding and hybridization prediction, Nucleic Acids Res 31:3406-15
Shekdar et al. (2021) Cell engineer method using fluorogenic oligonucleotide signaling probes and flow cytometry, Biotechnology Letters 43:949-58.

### BRIEF DESCRIPTION OF THE DRAWINGS

FIG. 1 is a schematic representation of expression vector pCP4-N.

FIG. 2 is a schematic representation of expression vector pCP4-N1.

FIG. 3 is a schematic representation of expression vector pCP4-N2.

FIG. 4 is a schematic representation of expression vector pCP4-N3.

FIG. 5 is a schematic representation of expression vector pCP5-P.

FIG. 6 is a schematic representation of expression vector pCP5-P1.

FIG. 7 is a schematic representation of expression vector pCP5-P2.

FIG. 8 is a schematic representation of expression vector pCP5-P3.

FIG. 9 is a schematic representation of expression vector pCP4-H.

FIG. 10 is a schematic representation of expression vector pCP4-H1.

FIG. 11 is a schematic representation of expression vector pCP4-H2.

FIG. 12 is a schematic representation of expression vector pCP4-H3.

FIG. 13 is a schematic representation of expression vector pCP4-Z.

FIG. 14 is a schematic representation of expression vector pCP4-Z1.

FIG. 15 is a schematic representation of expression vector pCP4-Z2.

FIG. 16 is a schematic representation of expression vector pCP4-Z3.

FIG. 17 is a schematic representation of expression vector pCP5-B.

FIG. 18 is a schematic representation of expression vector pCP5-B1.

FIG. 19 is a schematic representation of expression vector pCP5-B2.

FIG. 20 is a schematic representation of expression vector pCP5-B3.

FIG. 21A shows RNA sequences corresponding to three plasmid or vector-encoded detection tags termed detection tags A, B and C. Shading indicates sequence variations between detection tag A and either detection tag B or C. Detection tags A, B and C vary from each other by at least 50% within the 24-nucleotide region indicated.

FIG. 21B shows predicted mRNA folding of detection tags A, B and C based on the mFold algorithm.

FIGS. 21C-E show representative results for the multiplexed detection of the three detection tags expressed in cells co-transfected with expression plasmids each comprising an expression cassette encoding a detection tag using differentially-labeled signaling probes directed to the 24-nucleotide region of the detection tags. FIG. 21C shows detection of a detection tag by a corresponding signaling probe in transfected cells (test cells, left panel) vs. control-transfected cells (control cells, right panel) using fluorescence microscopy. FIG. 21D shows cell cytometric analysis for the detection of detection tags A and B using differentially-labeled signaling probes corresponding to detection tags A, B and C in transfected cells (test cells) versus control-transfected cells (control cells), as indicated. Fluorescence above background corresponding to signaling probes directed to detection tags A and B is shown in the left and center panels, respectively. Background fluorescence corresponding to control signaling probe directed to detection tag C is shown in the right panel. X-axis: fluorescence signal corresponding to the indicated probe, Y-axis: normalized cell count. FIG. 21E shows 2D dot plots for the simultaneous detection of detection tags A and B in transfected cells (left panel) versus control-transfected cells (right panel). Percentages of cells falling in each quadrant are indicated. X-axis and Y-axis: fluorescence signal corresponding to the indicated probe.

FIG. 21F shows the functional cell-based response of the heterodimeric human T1R2/T1R3 GPCR sweet taste receptor cell line made using gene-specific probes (left panel) to sucrose. The functional cell-based response of the NaV1.7-αβ1β2 cell line to a NaV1.7 inhibitor CC8464 (U.S. Pat. No. 9,458,118), is shown in the center panel (n=7). X-axis: CC8464 concentration, Y-axis: fraction of normalized current blocked using a whole-cell patch-clamp assay. Functional fluorescent membrane potential cell-based response of a combinatorial panel of cell lines for four GABA_{A} receptor ion channel subunit combinations including α1β3γ2s, α2β3γ2s, α3β3γ2s or α5β3γ2s to GABA was also generated and is shown in the right panel.

FIG. 22A shows a dose-response curve for the CFTR cell line to Forskolin resulting in an EC50 value of 256 nM (left panel) and that a CFTR-A508 cell line enables a functional cell-based assay for CFTR-A508 to 30 μM Forskolin+100 μM IBMX (3-isobutyl-1-methyl xanthine) versus DMSO+Buffer in the absence of any added trafficking correctors (right panel).

FIG. 22B shows the functional cell-based responses of multiple Chromovert® Technology-enabled CFTR cell lines compared to transiently-transfected cells. One Chromovert Technology-enabled cell line (Clone 1) compared to transient-transfected cells is shown (left panel). Clone 1 is compared to additional Chromovert-Technology enabled cell lines (right panel).

FIG. 23A shows ENaC is cytotoxic when expressed in transfected cell cultures (left panel) compared to control cells (right panel) as visualized by phase-contrast microscopy.

FIG. 23B shows a functional cell-based assay selective for αβγ-ENaC using cells transiently-transfected cells. X-axis: time, Y-axis, Arrows: addition of solution to increase sodium by 75 mM (left panel) and a functional cell-based response of a Chromovert® Technology enabled an αβγ-ENaC cell line in response to changes in sodium concentration (center panel) and inhibition of the response to a 75 mM increase in sodium concentration by amiloride (right panel) using the cell-based assay selective for αβγ-ENaC activity.

FIG. 23C shows limited proteolysis of the ENaC cell line to achieve functionally distinct proteolyzed variants of αβγ-ENaC in the context of the cell-based assay (n=9).

FIG. 23D shows high throughput screening (HTS) of a compound library against non-proteolyzed vs. proteolyzed ENaC variants resulted in largely non-overlapping hits. Only four of the top 500 hits identified in HTS of proteolyzed αβγ-ENaC were also identified among the top 500 hits identified in HTS of the same compound library against non-proteolyzed ENaC, as rank-ordered.

FIG. 23E shows The chemical structure (left panel) and human sensory taste testing results (STE1, right panel) for 4-propylphenyl 2-furoate, one potentiator of proteolyzed αβγ-ENaC that was identified during HTS and assessed and confirmed in human sensory studies to enhance the salty taste of sodium chloride (right panel). The sensory studies used pairwise comparisons of 45 mM NaCl+15 pg STE1/mL using a human sensory taste testing protocol approved by an institutional review board, 13 out of 15 participants selected the sample containing STE1 as more salty.

### DETAILED DESCRIPTION

The present disclosure describes technology utilizing novel expression vectors, fluorogenic oligonucleotide signaling probes and cells expressing one or more sequences of interest and kits comprising one or more of the foregoing.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, or method that comprises a list of elements is not necessarily limited to only those elements but may include other elements not expressly listed or inherent to such composition, mixture, process or method. For example, the sequence of interest in a cell that comprises a sequence of interest can be a sequence that is endogenous to the cell or a sequence that is introduced into the cell wherein the introduced sequences can be contained in the genome, chromosomes, nucleus or cytoplasm of the cell. A sequence of interest that is comprised in a cell may be permanently, stably, or semi-permanently or semi-stably contained in the cell over time. For instance, a cell comprising a sequence of interest may contain up to 100%, 90%, 85%, 80%, 75%, 70%, 65%, 60%, 55%, 50% or less than 50% of the level, amount or copy number of the sequence of interest or expression products encoded by the sequence of interest over time, for instance over 1 to 7 days, 1 to 5 weeks, 1 to 12 months or up to 1 to 5 years of continuous cell culture of the cells in the presence or absence of selective pressure wherein the selective pressure is designed to maintain the level, amount or copy number of the sequence of interest and/or its expression products. The sequence of interest in a cell produced to comprise a sequence of interest can be present at the DNA or chromosomal level only. The sequence of interest in a cell produced to comprise a sequence of interest can be present at the DNA or chromosomal level wherein it can additionally be expressed and present at the RNA and/or protein level, for instance as mRNA and/or as a polypeptide.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claims. The term “consisting essentially of” occupies a middle ground between “comprising” and “consisting of”.

Where applicants have defined a disclosure or a portion thereof with an open-ended term such as “comprising,” it should be readily understood that (unless otherwise stated) the description should be interpreted to also describe such a disclosure using the terms “consisting essentially of” or “consisting of.”

Further, unless expressly stated to the contrary, “or” refers to an inclusive or and not to an exclusive or. For example, a condition A or B is satisfied by any one of the following: A is true (or present), and B is false (or not present), A is false (or not present) and B is true (or present), and both A and B are true (or present).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this disclosure. Any methods and materials similar or equivalent to those described herein can be used in the practice or testing of the present teachings. Numeric ranges provided herein are inclusive of the numbers defining the range.

Definitions unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference. Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

The term “polynucleotide” is intended to encompass a singular nucleic acid as well as plural nucleic acids, and refers to a nucleic acid molecule or construct, e.g., messenger RNA (mRNA) or plasmid DNA (pDNA). A polynucleotide can contain the nucleotide sequence of the full-length cDNA sequence, or a fragment thereof, including the untranslated 5′ and 3′ sequences and the coding sequences. The polynucleotide can be composed of any polyribo-nucleotide or polydeoxyribonucleotide, which may be unmodified RNA or DNA or modified RNA or DNA. For example, polynucleotides can be composed of single- and double stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double stranded or a mixture of single- and double-stranded regions. “Polynucleotide” embraces chemically, enzymatically, or metabolically modified form.

As used herein, a “nucleic acid” refers to two or more deoxyribonucleotides and/or ribonucleotides covalently joined together in either single or double-stranded form. By “recombinant nucleic acid” is meant a nucleic acid of interest that is free of one or more nucleic acids (e.g., a sequence of interest or a gene of interest) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences.

By “heterologous nucleic acid” is meant a nucleic acid sequence derived from a different organism, species or strain than the host cell. In some embodiments, the heterologous nucleic acid is not identical to a wild-type nucleic acid that is found in the same host cell in nature. A polynucleotide sequence may be referred to as “isolated,” in which it has been removed from its native environment. For example, a heterologous polynucleotide encoding a polypeptide or polypeptide fragment having 5-Methyl D-tryptophan transferase activity contained in a vector is considered isolated for the purposes of the present disclosure. Further examples of an isolated polynucleotide include recombinant polynucleotides maintained in heterologous host cells or purified (partially or substantially) polynucleotides in solution. Isolated polynucleotides or nucleic acids according to the present disclosure further include such molecules produced synthetically. An isolated polynucleotide fragment in the form of a polymer of DNA may be comprised of one or more segments of cDNA, genomic DNA or synthetic DNA. The term “gene” refers to a nucleic acid fragment or sequence of interest that is capable of being expressed as a specific protein, optionally including regulatory sequences preceding (5′ non-coding sequences) and following (3′ non-coding sequences) the coding sequence. “Native gene” refers to a gene sequence of interest or as found in nature with its own regulatory sequences. “Endogenous gene” refers to a native gene sequence of interest or in its natural location in the genome of an organism. A “heterologous gene” refers to a gene sequence of interest or not normally found in the host organism, but that is introduced into the host organism by gene transfer. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. Foreign genes can comprise native genes inserted into a non-native organism, or chimeric genes. In another example, a heterologous gene can be a gene reintroduced into the source organism in a location that is different from that in the unaltered host organism.

As used herein the term “coding region” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. As used herein, an “expression control sequence” means a nucleic acid sequence that directs transcription of a nucleic acid of interest. An expression control sequence can be a promoter, such as a constitutive or an inducible promoter, or an enhancer. An expression control sequence can be “native” or heterologous. A native expression control sequence is derived from the same organism, species, or strain as the gene being expressed. A heterologous expression control sequence is derived from a different organism, species, or strain as the gene being expressed. An “inducible promoter” is a promoter that is active under environmental or developmental regulation.

By “operably linked” is meant a functional linkage between a nucleic acid expression control sequence (such as a promoter) and a second nucleic acid sequence, wherein the expression control sequence directs transcription of the nucleic acid corresponding to the second sequence. As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. A polypeptide may be derived from a natural biological source or produced by recombinant technology, but is not necessarily translated from a designated nucleic acid sequence. It may be generated in any manner, including by chemical synthesis. As used herein, the term “variant” refers to a polypeptide differing from a specifically recited polypeptide of the disclosure by amino acid insertions, deletions, mutations, and substitutions, created using, e.g., recombinant DNA techniques, such as mutagenesis. Guidance in determining which amino acid residues may be replaced, added, or deleted without abolishing activities of interest, may be found by comparing the sequence of the particular polypeptide with that of homologous polypeptides, e.g., yeast or bacterial, and minimizing the number of amino acid sequence changes made in regions of high homology (conserved regions) or by replacing amino acids with consensus sequences. By “heterologous polypeptide” is meant a polypeptide encoded by a nucleic acid sequence derived from a different organism, species, or strain than the host cell. In some embodiments, a heterologous polypeptide is not identical to a wild-type polypeptide that is found in the same host cell in nature.

The terms “host cell” or “host microorganism” refer to a cell culture, cell line, cells of a cell culture or line, a mixed population of cells, mammalian cells, a mammalian cell culture, cells, a cell line or cell cultures obtained from a repository including ATCC or ECACC, including CHO and HEK 293 and HEK 293T cells and/or microorganism. A host cell may be capable of receiving any sequence of interest including a foreign or heterologous sequence of interest or genes of interest. A host cell may be capable of receiving any sequence of interest including a foreign or heterologous sequence of interest or genes of interest and of containing, comprising and/or expressing those sequences of interest or genes of interest to produce an expression or active gene product including mRNA or polypeptide sequence.

In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products. Moreover, the skilled artisan recognizes that substantially similar sequences encompassed by this disclosure are also defined by their ability to hybridize, under stringent conditions (0.1×SSC, 0.1% SDS, 65° C. and washed with 2×SSC, 0.1% SDS followed by 0.1×SSC, 0.1% SDS), with the sequences exemplified herein. Preferred substantially similar nucleic acid fragments of the instant disclosure are those nucleic acid fragments whose DNA sequences are at least 80% identical to the DNA sequence of the nucleic acid fragments reported herein. More preferred nucleic acid fragments are at least 90% identical to the DNA sequence of the nucleic acid fragments reported herein. Most preferred are nucleic acid fragments that are at least 95% identical to the DNA Sequence of the nucleic acid fragments reported herein. A nucleic acid fragment is “hybridizable” to another nucleic acid fragment, such as a cDNA, genomic DNA, or RNA, when a single stranded form of the nucleic acid fragment can anneal to the other nucleic acid fragment under the appropriate conditions of temperature and Solution ionic strength. Hybridization and washing conditions are well known and exemplified in Sambrook, J., Fritsch, E. F. and Maniatis, T. Molecular Cloning. A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic Strength determine the “Stringency” of the hybridization. For preliminary Screening for homologous nucleic acids, low Stringency hybridization conditions, corresponding to a Tm of 55, can be used, e.g., 5×SSC, 0.1% SDS, 0.25% milk, and no formamide; or 30% formamide, 5×SSC, 0.5% SDS. Moderate stringency hybridization conditions correspond to a higher Tm, e.g., 40% formamide, with 5x or 6×SSC. Hybridization requires that the two nucleic acids contain complementary sequences, although depending on the Stringency of the hybridization, mismatches between bases are possible. The appropriate Stringency for hybridizing nucleic acids depends on the length of the nucleic acids and the degree of complementary sequences, although depending on the Stringency of the hybridization, mismatches between bases are possible. The appropriate Stringency for hybridizing nucleic acids depends on the length of the nucleic acids and the degree of complementation, variables well known in the art. The greater the degree of Similarity or homology between two nucleotide sequences, the greater the value of Tm for hybrids of nucleic acids having those sequences. The relative stability (corresponding to higher Tm) of nucleic acid hybridization decreases in the following order: RNA:RNA, DNA:RNA, DNA:DNA. For hybrids of greater than 100 nucleotides in length, equations for calculating Tm have been derived (see Sambrook et al., Supra, 9.50-9.51). For hybridization with Shorter nucleic acids, i.e., oligonucleotides, the position of mismatches becomes more important, and the length of the oligonucleotide determines its specificity (see Sambrook et al., Supra, 11.7-11.8). In one embodiment the length for a hybridizable nucleic acid is at least about 10 nucleotides. Preferable a minimum length for a hybridizable nucleic acid is at least about 15 nucleotides, more preferably at least about 20 nucleotides, and most preferably the length is at least 30 nucleotides. Furthermore, the skilled artisan will recognize that the temperature and wash Solution Salt con centration may be adjusted as necessary according to factors Such as length of the probe. A “substantial portion” refers to an amino acid or nucleotide sequence which comprises enough of the amino acid sequence of a polypeptide or the nucleotide sequence of a sequence of interest or gene to afford putative identification of that polypeptide or sequence of interest or gene, either by manual evaluation of the Sequence by one skilled in the art, or by computer-automated Sequence comparison and identification using algorithms. Such as BLAST (Basic Local Alignment Search Tool; Altschul et al., J. Mol. Biol. 215:403-410 (1993); see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene-specific oligonucleotide probes comprising 20-30, 20-40, 30-50, 25 to 70, 20 to 80, 30 to 150, 30 to 200 or over 200 contiguous nucleotides may be used in sequence-dependent methods of gene identification or identification of a sequence of interest or (e.g., Southern hybridization) and isolation (e.g., in Situ hybridization of bacterial colonies or bacteriophage plaques). In addition, short oligonucleotides of 12-15, 12 to 30, or more than 30 bases may be used as amplification primers in PCR in order to obtain a particular nucleic acid molecule comprising the primers. Accordingly, a “Substantial portion” of a nucleotide sequence comprises enough of the sequence to afford Specific identification and/or isolation of a nucleic acid molecule comprising the sequence. The instant specification teaches partial or complete amino acid and nucleotide sequences encoding one or more particular proteins. The skilled artisan, having the benefit of the sequences as reported herein, may now use all or a Substantial portion of the disclosed sequences for the purpose known to those skilled in the art. Accordingly, the instant disclosure comprises the complete sequences as reported in the accompanying Sequence Listing, as well as substantial portions of those sequences as defined above.

The term “complementary” describes the relationship between nucleotide bases that are capable to hybridizing to one another. For example, with respect to DNA, adenosine is complementary to thymine and cytosine is complementary to guanine. Accordingly, the instant disclosure also includes isolated nucleic acid molecules that are complementary to the complete sequences as reported in the accompanying Sequence Listing as well as those substantially similar nucleic acid sequences. Moreover, the instant disclosure includes novel plasmids encoding a spacer sequence and a detection tag wherein any nucleic acid sequence of interest may be subcloned into the plasmid. The present disclosures includes any plasmid comprising a spacer sequence, detection tags and a sequence of interest.

The term “percent identity”, as known in the art, is a relationship between two or more polypeptide sequences or two or more polynucleotide sequences, as determined by comparing the sequences. In the art, “identity also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology, Lesk, A. M., Ed., Oxford University Press: New York, 1988, Biocomputing. Informatics and Genome Projects; Smith, D. W., Ed.; Academic Press: New York, 1993, Computer Analysis of Sequence Data, Part I; Griffin, A. M. and Griffin, H. G., Eds.; Humana Press: New Jersey, 1994, Sequence Analysis in Molecular Biology, von Heinje, G., Ed.; Academic Press: New York, 1987; and Sequence Analysis Primer, Gribskov, M. and Devereux, J., Eds.; Stockton Press: New York, 1991. Preferred methods to determine identity are designed to give the largest match between the Sequences tested.

Methods to determine identity and similarity are codified in publicly available computer programs. Preferred computer program methods to determine identity and Similarity between two Sequences include, but are not limited to, the GCG Pileup program found in the GCG program package, using the Needleman and Wunsch algorithm with their Standard default values of gap creation penalty=12 and gap extension penalty=4 (Devereux et al., Nucleic Acids Res. 12:387-395 (1984)), BLASTP, BLASTN, and FASTA (Pearson et al., Proc. Natl. Acad. Sci. USA 85:2444-2448 (1988). The BLASTX program is publicly available from NCBI and other sources (BLAST Manual, Altschul et al., Natl. Cent. Biotechnol. Inf, Natl. Library Med. (NCBI NLM) NIH, Bethesda, Md. 20894; Altschul et al., J. Mol. Biol. 215:403410 (1990); Altschul et al., “Gapped BLAST and PSI-BLAST: a new generation of protein database search programs”, Nucleic Acids Res. 25:3389-3402 (1997)). Another preferred method to determine percent identity, is by the method of DNASTAR protein alignment protocol using the Jotun-Hein algorithm (Hein et al., Methods Enzymol. 183:626-645 (1990)). Default parameters for the Jotun Hein method for alignments are: for multiple alignments, gap penalty=11, gap length penalty=3; for pairwise alignments ktuple=6. As an illustration, by a polynucleotide having a nucleotide Sequence having at least, for example, 95% “identity” to a reference nucleotide sequence it is intended that the nucleotide sequence of the polynucleotide is identical to the reference sequence except that the poly nucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having an amino acid sequence having at least, for example, 95% identity to a reference amino acid Sequence is intended that the amino acid sequence of the polypeptide is identical to the reference sequence except that the polypeptide sequence may include up to five amino acid alterations per each 100 amino acids of the reference amino acid. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

The term “percent homology” refers to the extent of amino acid sequence identity between polypeptides. When a first amino acid sequence is identical to a second amino acid sequence, then the first and second amino acid sequences exhibit 100% homology. The homology between any two polypeptides is a direct function of the total number of matching amino acids at a given position in either sequence, e.g., if half of the total number of amino acids in either of the two sequences are the same then the two sequences are said to exhibit 50% homology.

“Codon degeneracy” refers to divergence in the genetic code permitting variation of the nucleotide sequence without effecting the amino acid sequence of an encoded polypeptide. The skilled artisan is well aware of the “codon bias’ exhibited by a specific host cell in usage of nucleotide codons to specify a given amino acid. Therefore, when synthesizing a sequence of interest or gene for improved expression in a host cell, it is desirable to design the sequence of interest or gene such that its frequency of codon usage approaches the frequency of preferred codon usage of the host cell.

Modifications to the sequence, such as deletions, insertions, or substitutions in the sequence which produce silent changes that do not substantially affect the functional properties of the resulting protein molecule are also contemplated. For example, alteration in the sequence of interest or gene sequence which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as Valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product. Nucleotide changes which result in alteration of the N-terminal and C-terminal portions of the protein molecule would also not be expected to alter the activity of the protein. In some cases, it may in fact be desirable to make mutants of the sequence in order to study the effect of alteration on the biological activity of the protein. Each of the proposed modifications is well within the routine skill in the art, as is determination of retention of biological activity in the encoded products. Moreover, the skilled artisan recognizes that sequences encompassed by this disclosure are also defined by their ability to hybridize, under stringent conditions (0.1×SSC, 0.1% SDS, 65° C.), with the sequences exemplified herein.

Modifications to the sequence, such as deletions, insertions, substitutions, mutations or recombination in the sequence which produce changes that do substantially affect the functional properties of the resulting protein molecule are also contemplated. For instance, cells comprising altered variants of a sequence of interest may be generated to detect variants or their expression products comprising improved function or characteristics.

The term “expression” refers to the transcription and/or translation of a nucleic acid sequence of interest. Expression includes the transcription of a DNA nucleic acid sequence into mRNA. Expression includes the translation of an mRNA into a polypeptide. Expression includes the transcription of a DNA nucleic acid sequence into an RNA product that is not translated into a polypeptide. The expression of a sequence of interest in cells produced to comprise a sequence of interest according to the present disclosure can be characterized, for instance, the level of expression of mRNA or polypeptides can be measured and the consistency or stability of the expression level of the mRNA or polypeptides over time in culture can be measured, including using a functional cell-based assay. The present disclosure includes the production of cells comprising a sequence of interest wherein the expression level of the sequence of interest is stable over time in cell culture. As used herein, the term expression can also mean the transcription and translation to gene product from a gene coding for the sequence of the gene product.

The terms “plasmid”, “vector”, and “cassette” refer to an extra chromosomal element often containing, carrying or comprising one or more sequences of interest or genes which are not part of the central metabolism of the cell, and usually in the form of circular double-stranded DNA molecules. Such elements may be autonomously replicating sequences, genome integrating sequences, phage or nucleotide sequences, linear or circular, of a single- or double stranded DNA or RNA, derived from any source, in which a number of nucleotide Sequences have been joined or recombined into a unique construction which is capable of introducing a promoter fragment and DNA sequence for a selected sequence of interest or gene product along with appropriate 3′ untranslated sequence into a cell. “Transformation cassette” refers to a specific vector containing a foreign gene and having elements in addition to the foreign gene that facilitates transformation of a particular host cell. “Expression cassette” refers to a specific vector containing a sequence of interest or foreign gene and having elements in addition to the sequence of interest or foreign gene that may optionally allow for enhanced expression of that sequence of interest or gene in a foreign host.

As used herein a “mixed cell population” may be a cell population of cell types of different origin, a cell population of cells of one cell type that are genetically heterologous. A mixed population of cells can be used as host cells or cell lines. A mixed population of cells can mean a cell culture or culture of cells, including a cell line, a clonal cell line, an immortalized cell line, a transformed cell line, cells or cell lines obtained from ATCC, ECACC or a laboratory or repository of cells, or a mixture or combination of any of these, wherein at least one of the cells of the culture varies from at least one other cell in the culture. A mixed population of cells can include cell cultures wherein the cells of the cell culture can vary genetically, phenotypically, functionally, biochemically, by their expression of one or more endogenous or introduced genes or sequences of interest or by any property that may be measured using one or more laboratory tests. A mixed population of cells can include cell cultures wherein the cells of the cell culture can vary with respect to their capacity for DNA, RNA or protein expression, production, assembly and/or folding. A mixed population of cells can include cell cultures wherein the cells of the cell culture can vary with respect to their capacity for production of proteins, drug targets, biologics, biologic drugs and/or gene therapy agents. A mixed population of cells can include cell cultures wherein the cells of the cell culture can vary with respect to their capacity for production, post-translational modification, folding, membrane integration or secretion of membrane proteins, secreted proteins or virus particles, including multi-subunit proteins or proteins comprising more than one polypeptide. A mixed population of cells can include cell cultures wherein the cells of the cell culture can vary with respect to their capacity for production and assembly of virus particles, including adeno-associated virus, recombinant adeno-associated virus, herpes virus, lentivirus, DNA or RNA virus, retrovirus, HIV or coronavirus, including SARS, MERS, SAR-CoV-2, including virus for use in drug discovery, vaccine development and/or gene therapy. A mixed populations of cells includes cell cultures wherein at least one cell that has a genome that is different than the genome of at least one other cell or at least one cell that has a different gene expression profile compared to at least one other cell. A mixed populations of cells includes cell cultures that are exposed to at least one sequence of interest wherein at least one cell in the population comprises or expresses at least one of the sequences of interest at a different level compared to at least one other cell of the cell culture. A mixed population of cells includes a clonal cell line comprising identical cells wherein at least one of the identical cells is altered or changed. A mixed population of cells includes cell cultures within which individual cells with desired properties may be detected and/or isolated, including using signaling probes. A mixed population of cells includes cell cultures wherein individual cells differ with respect to their expression levels of one or more sequences of interest wherein cells with desired expression levels or no expression of the one or more sequences of interest may be detected and/or isolated, including using signaling probes. In some embodiments, a mixed population of cells comprises cells that include one or more sequences of interest and cells that do not include one or more sequences of interest.

As used herein, a “signaling probe” is a molecule that may be used to report the presence of a sequence of interest. A signaling probe may be an oligonucleotide that comprises one or more fluorophores. A signaling probe may be an oligonucleotide that comprises one or more fluorophores and one or more quenchers. A signaling probe may be a molecular beacon. A signaling probes may be an oligonucleotide that undergoes a fluorogenic conformational change in the presence of target sequence of interest. A signaling probe may be directed to detect, bind to, or hybridize with a sequence of interest, including a nucleic acid, chromosomal, gene, plasmid, vector, DNA, detection tag, RNA, mRNA, nuclear or cytoplasmic sequence. A signaling probe may be a fluorescent or fluorogenic oligonucleotide. A signaling probe may be used to detect and/or isolate cells of a mixed population of cells that comprise or express a sequence of interest. A signaling probe may be used in combination with one or more additional differentially fluorescently labeled signaling probes for the detection of at least two different sequences of interest. A signaling probe may be introduced into cells. A signaling probe may be detected using flow cytometry, fluorescence-activated cell sorting (FACS®) or fluorescence microscopy. A signaling probe may comprise natural nucleic acid bases, including DNA or RNA bases, natural nucleic acid chemistry, artificial nucleic acid bases, artificial nucleic acid chemistry, nucleic acid bases that are chemically modified, nucleic acid base that are covalently linked to other molecules and peptide nucleic acid sequences.

As used herein, the term “sequence of interest” or “sequence” refers to a DNA or RNA nucleic acid sequence. A sequence of interest can be a polynucleotide, a polynucleotide sequence, an isolated polynucleotide, an isolated polynucleotide sequence, a heterologous nucleic acid, an endogenous nucleic acid, a coding region, a regulatory sequence, a suitable regulatory sequence, a expression control sequence or a functional nucleic acid sequence. A sequence of interest can be a genetic sequence including a chromosome or chromosomal sequence, a genomic sequence, a gene (that is, a sequence of DNA that encodes a polypeptide), a gene of interest, an exon, an intron, cDNA, plasmid or vector sequence. A sequence of interest that is an RNA can be any type of RNA, including a messenger RNA or mRNA, an alternatively spliced RNA, a ribosomal RNA or rRNA, a transfer RNA or tRNA, an antisense RNA or a ribozyme. A sequence of interest can have a function or activity including a hybridization, binding, enzymatic, or localization activity. A sequence of interest can elicit an immune or other biological response. A sequence of interest may be used to modify the genome of a cell or organism. A sequence of interest may encode a protein, amino acid sequence, polypeptide, peptide, peptide nucleic acid, receptor, enzyme, functional protein, membrane protein, structural protein, signaling protein, signal peptide, drug target, biologic, biologic drug, antibody, virus or gene therapy agent. A sequence of interest may be an oligonucleotide. A sequence of interest may comprise 5 to 15, 10 to 100, 50 to 250, 100 to 500, 250 to 1,000, 500 to 2,500, 1,000 to 5,000, 2,500 to 10,000, 5,000 to 25,000, 10,000 to 100,000, 50,000 to 250,000, 100,000 to 500,000, 250,000 to 1,000,000, 500,000 to 5,000,000 or greater than 5,000,000 DNA or RNA residues, nucleotides or bases. A sequence of interest may derive from or correspond to any cell, virus, bacterium, microbe, yeast, plant, insect, animal, invertebrate, vertebrate, mammal or organism. A sequence of interest may be a naturally-occurring sequence, a modification of a naturally-occurring sequence, an artificial sequence or a sequence created through in silico design, in vitro evolution, mutation, shuffling, subcloning or recombination. A sequence of interest or expression products of the sequence of interest may be detected using a signaling probe including a fluorogenic probe and/or flow cytometry. A sequence of interest may be a detection tag or may correspond to a detection tag sequence. A sequence of interest may be introduced into cells, including living cells, mammalian cells, and cells of a cell culture or cell lines. A sequence of interest may be introduced into organisms including viruses, microbes, plants, insects and animals.

As used herein, the term “expression plasmid” refers to any vector that may be used to introduce or deliver a sequence of interest into a virus, microbe, cell, host cell, mixed population of cells, cell culture, cell line or mammalian cell. An expression plasmid can encode a sequence of interest, a nucleic acid, genomic, DNA, cDNA, plasmid, detection tag or RNA sequence. Expression plasmids may encode a sequence of interest wherein the sequence is not expressed in cells or wherein the sequence is expressed in cells. Cells comprising an expression plasmid may be detected and/or isolated using signaling probes, including fluorogenic signaling probes, directed to the sequence of interest or to a detection tag or other nucleic acid sequence that is associated with the sequence of interest. Signaling probes may be directed to the expression plasmid or to expression products expressed from the expression plasmids. Expression plasmids may comprise selectable markers including sequence of interest or genes that confer resistance to antibiotics and/or drugs.

The capacity of cells to produce protein may be increased under certain conditions, for instance when unfolded proteins in the ER trigger the unfolded protein response, termed “UPR”. The present disclosure provides for the creation of cells that express proteins to trigger, regulate, maintain or contribute to UPR as a means to upregulate the protein production capacity of cells for use to create new host cell lines for biologics production (e.g., antibody and protein drugs) and/or the production of viruses (e.g., rAAV) for use in gene, genetic or cell therapy. Sustained UPR can be cytotoxic in cells. The present disclosure provides for the creation of cells that exhibit decreased or no cytotoxicity under conditions that elicit sustained UPR, including following the introduction of one or more sequences of interest into cells.

One or more sequences of interest, genes or RNAs (including mutated, spliced, and processed forms) and expression products encoded by these that may be involved in biological pathways (e.g., those involved in the unfolded protein response (UPR), cell viability, protein production, folding, assembly, modification, glycosylation, proteolysis, secretion, integration into membrane of a cell, cell surface presentation or a combination of these) including, but not limited to: ATF6a spliced, IREI a, IREI b, PERCDC, ATF4, YYI, NF-YA, NF-YB, NF-YC, XBP1 spliced, and EDEM1 (UPR genes); NRF2, HERP, XIAP, GADD34, PPI a, b and g, and DNAJC3 (switch-off genes); BLIMP-1 and XBP1 spliced (genes expressed in B cells); CRT (CaBP3), CNX, ERp57 (PDIA3), BiP, BAP, ERdj3, CaBP1, GRP94 (CaBP4), ERp72 (PDIA4), and cyclophilin B (folding/secretion genes—Class 1 Chaperones); BiP, BAP, ERdj3, CaBP1, GRP94 (CaBP4), ERp72 (PDIA4) and cyclophilin B (Class 2 Chaperones); SDF2-L (glycosylation gene); EROI a and b, ERAD, mannosidase 1, HRD1 (oxidation genes); STC1 and 2, SERCA1 and 2, COD1 (calcium pumps); INO1, SREBP1 DC, SREBP2DC, and PYC (lipogenesis/metabolism genes); Sec61 Pa,b and g (transport/membrane integration genes); and Bcl-2sp, Bcl-xL, Bim, Ku70, VDAC2, BAP31 and 14-3-3 (cell viability/anti-apoptosis genes).

In certain embodiments, a cell is engineered to express at least 2, 5, 10, 15, 20, 25, 30, 40, 50, 75, 100, or 150 sequences of interest including one or more protein expression accessory factors. Illustrative sequences of interest or protein expression accessory factors include: proteins that regulate the unfolded protein response (UPR) and genes that encode proteins that are regulated in the UPR (e.g. Λ ATF6α (spliced), IRE1 α, IRE1 β, PERCΔC, ATF4, YYI, NF-YA, NF-YB, NF-YC, XBP1 (spliced), EDEM1); genes that encode proteins that switch-off the apoptotic pathway induced by the UPR (e.g. Λ NRF2, HERP XIAP, GADD34, PPIα, PPIβ, PPIγ, DNAJC3); genes that encode proteins that affect the growth of cells, the viability of cells, cell death, and cell size; B-cell genes (e.g., BLIMP-1, XBP1 (spliced)); genes that encode proteins involved in protein transport (e.g., Sec61 Pa, Sec61 PP, Sec61 PT); genes that encode proteins involved in glycosylation (e.g., SDF2-L); genes that encode proteins involved in oxidation (e.g., ERO1 α, ERO1 β); genes that encode anti-apoptotic proteins (e.g., Bcl-2sp, BcI-xL, Bim trunk. Mut., Ku70, 14-3-3q mut., VDAC2, BAP31 mut.); genes that encode proteins implicated in endoplasmic reticulum-associated degradation (e.g., mannosidase 1, HRD1); genes that encode proteins involved in calcium transport (e.g., STC1, STC2, SERCA1, SERCA2, COD1); genes that encode proteins implicated in lipogenesis/metabolism (e.g., INO1, PYC, SREBP1 ΔC, SREBP2ΔC); and genes that encode proteins implicated in protein folding and secretion (e.g. Λ CRT (CaBP3), CNX, ERp57 (PDIA3), BiP, BAP, ERdj3, CaBP1, GRP94 (CaBP4), ERp72 (PDIA4), cyclophilin B), protein assembly, the integration of proteins into membranes, cell surface presentation of proteins, and post-translational modification of proteins.

In a specific embodiment, the cells are engineered to express any one or a combination of sequences of interest implicated in the UPR. In another specific embodiment, the cells are engineered to express any one or a combination of sequences of interest implicated in the UPR as well as at least one other gene that encodes a protein suspected or known to be beneficial for protein production or cell viability. Sequences of interest that regulate UPR or are regulated in UPR; sequences of interest that alter cell growth, viability, apoptosis, cell death, cell size; sequences of interest encoding chaperones or factors implicated in protein folding, assembly, membrane integration, cell surface presentation or secretion, post-translational modification including glycosylation/phosphoylation/proteolysis can be used. In certain embodiments, a protein expression accessory factor alters a cell physiological property. Identification of cells that express one or a combination of sequences of interest that encode proteins suspected or known to be beneficial for protein production or cell viability can be accomplished using the methods described herein, e.g., signaling probes that bind to target sequences in the sequences of interest or genes/mRNA of interest could be generated and the presence of the sequences of interest or gene/mRNA of interest then could be verified by FACS analysis, which may also be used to isolate positive cells.

In an illustrative embodiment, a cell is transfected with a first nucleic acid encoding the protein of interest and a second nucleic acid encoding a protein expression accessory factor; a fluorogenic oligonucleotide capable of detecting the transcript of the first nucleic acid and a fluorogenic oligonucleotide capable of detecting the transcript of the second nucleic acid are introduced into the cell; selection of a cell that expresses the protein of interest and the protein expression accessory factor. A cell line that expresses the protein of interest consistently and reproducibly can then be established. The cell line can be further tested for physiological properties related to protein expression as discussed above.

In one embodiment, cells are first engineered to express a protein expression accessory factor; cell lines expressing the protein expression accessory factor are established, cells of the cell line are then engineered to express a protein of interest. In another embodiment, cells are first engineered to express a protein of interest; cell lines expressing the protein of interest are established, cells of the cell line are then engineered to express a protein expression accessory factor.

In even another embodiment, cells are concurrently engineered to express a protein of interest and a protein expression accessory factor. Cell lines that express the protein of interest and/or the protein expression accessory factor consistently and reproducibly can then be established as described herein.

In one embodiment, creation of cells and cell lines comprising one or more plasmids for the production of viruses for use in gene, genetic engineering or cell therapy, including a plasmid comprising genetic material for packing into the virus particles, a plasmid comprising viral genes, and, optionally, a third plasmid comprising accessory or helper genes to facilitate or required for viral production, forms part of the present disclosure. As used herein, the plasmids can additionally comprise detection tags. In some embodiments, the plasmids comprising viral sequences of interest can include genes from a DNA virus, RNA virus, retrovirus, adeno-associated virus, adenovirus, lentivirus, herpes virus, HIV and coronavirus, including SARS, MERS, and SARS-CoV-2.

In one embodiment, creation of cells and cell lines comprising one or more plasmids for rAAV (recombinant adeno-associated virus) production, including a plasmid comprising genetic material for packing into virus particles, a plasmid comprising adeno-associated virus genes for rep and cap genes, and a third plasmid comprising adenovirus genes needed for production of the virus (e.g., E2A, E4, VA genes) form part of the present disclosure. rAAV is used in gene therapy applications. The virus is typically produced using transient transfection of three plasmids into mammalian cells, typically HEK 293T cells. Robust stable cell lines comprising one or more of the plasmids are challenging to produce. The methods of the present disclosure may be used to produce stable cell lines comprising one or more of the plasmids required to produce rAAV. The plasmids required to produce rAAV may comprise one or more detection tag sequences. Expression products encoded by these plasmids may be under the control of constitutive and/or inducible promotors.

AAV belongs to the Adenovirus genus and depends on a co-infecting helper virus (usually adenovirus) for productive infection to occur. Additionally, the recombinant AAV genome has two essential genes removed to prevent integration and replication to make AAV a safe and effective tool for gene delivery. Therefore, in order to generate more AAV particles, essential genes must be provided in trans. A triple transfection strategy for AAV packaging involves co-transfecting the packaging cell line (usually HEK 293T) with three plasmids: 1) the recombinant AAV plasmid containing the genetic payload to be packaged into virus particles for use in gene therapy, 2) a plasmid containing the essential AAV rep and cap genes, and 3) a third plasmid comprising one or more helper genes derived from adenovirus (e.g., E2A, E4, VA genes) supplying genes needed for virus production.

In one embodiment, creation of cells and cell lines comprising one or more plasmids comprising sequences of interest or genes encoding the human sensory, taste and odorant receptors, including sweet, bitter, salt, sour, umami, hot, pepper, cool, mint, fat, fatty acid, kokumi, mouth feel, touch, touch sensation, mechano sensation, pressure sensing and tingle receptor genes and odorant receptor genes.

In some embodiments a gene of interest is selected from ENaC, CFTR, Nav1.7, and GABA_{A}. In one embodiment, creation of cells and cell lines comprising one or more plasmids comprising ENaC, CFTR, Nav1.7, and GABA_{A} forms part of the present disclosure.

Construction of Recombinant Organisms. Recombinant organisms containing a gene of interest may be constructed using techniques well known in the art. Isolation of sequences of interest or genes. Methods of obtaining desired sequences of interest or genes from a genome are common and well known in the art of molecular biology. For example, if the sequence of the gene is known, suitable genomic libraries may be created by restriction endonuclease digestion and may be screened with probes complementary to the desired gene sequence. Once the sequence is isolated, the DNA may be amplified using standard primer directed amplification methods Such as polymerase chain reaction (PCR) (U.S. Pat. No. 4,683,202) to obtain amounts of DNA suitable for transformation using appropriate vectors.

Alternatively, cosmid libraries may be created where large segments of genomic DNA (3545 kb) may be packaged into vectors and used to transform appropriate hosts. Cosmid vectors are unique in being able to accommodate large quantities of DNA. Generally cosmid vectors have at least one copy of the cos DNA sequence which is needed for packaging and subsequent circularization of the foreign DNA. In addition to the cos sequence these vectors will also contain an origin of replication Such as Col. 1 and drug resistance markers such as a gene resistant to ampicillin or neomycin. Methods of using cosmid vectors for the transformation of suitable bacterial hosts are well described in Sambrook, J. et al., Supra.

Typically to clone cosmids, foreign DNA is isolated using the appropriate restriction endonucleases and ligated, adjacent to the cos region of the cosmid vector using the appropriate ligases. Cosmid vectors containing the linearized foreign DNA are then reacted with a DNA packaging vehicle such as bacteriophage. During the packaging process the cos sites are cleaved and the foreign DNA is packaged into the head portion of the bacterial viral particle. These particles are then used to transfect suitable host cells Such as E. coli. Once injected into the cell, the foreign DNA circularizes under the influence of the cos sticky ends. In this manner large Segments of foreign DNA can be introduced and expressed in recombinant host cells.

Host Cells. Suitable host cells useful for practicing the present disclosure include, but are not limited to: Chinese hamster ovary (CHO) cells, human embryonic kidney (HEK) cells, established neuronal cell lines, pheochromocytomas, neuroblastomas fibroblasts, rhabdomyosarcomas, dorsal root ganglion cells, NS0 cells, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), L-cells, HEK-293 (ATCC CRL1573) and PC12 (ATCC CRL-1721), HEK293T (ATCC CRL-1 1268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), Vero (ATCC CCL 81), Caco-2 (ATCC HTB 37), K562 (ATCC CCL 243), Jurkat (ATCC TIB-152), Per.C6 (Crucell, Leiden, The Netherlands), Huvec (ATCC Human Primary PCS 100-010, Mouse CRL 2514, CRL 2515, CRL 2516), HuH-7D12 (ECACC 01042712), 293 (ATCC CRL 10852), A549 (ATCC CCL 185), IMR-90 (ATCC CCL 186), MCF-7 (ATC HTB-22), U-2 OS (ATCC HTB-96), T84 (ATCC CCL 248), or any established cell line (polarized or nonpolarized) or any cell line available from repositories such as American Type Culture Collection (ATCC, 10801 University Blvd. Manassas, Va. 201 10-2209 USA) or European Collection of Cell Cultures (ECACC, Salisbury Wiltshire SP4 OJG England).

The host cells disclosed herein may be eukaryotic, prokaryotic, mammalian, human, primate, bovine, porcine, feline, rodent, marsupial, murine or other cells. The host cells disclosed herein include, but are not limited to nonmammalian cells, such as yeast, insect, fungus, and plant cells, and cells from lower eukaryotes and prokaryotes.

The host cells disclosed herein include, but are not limited to: transformed, oncogenically transformed, virally transformed, immortalized, conditionally transformed, explants, cells of tissue sections, animals, plants, fungi, protists, archaebacteria and eubacteria, mammals, birds, fish, reptiles, amphibians, and arthropods, avian, chicken, reptile, amphibian, frog, lizard, snake, fish, worms, squid, lobster, sea urchin, sea slug, sea squirt, fly, squid, hydra, arthropods, beetles, chicken, lamprey, ricefish, zebra finch, pufferfish, and zebrafish cells.

Vectors. The present disclosure provides a variety of vectors and transformation and expression cassettes suitable for the cloning, transformation and expression of a gene of interest into a suitable host cell. Virtually any promoter capable of driving genes of interest is suitable for the present disclosure including but not limited to Termination control regions may also be derived from various genes native to the preferred hosts. Optionally, a termination site may be unnecessary. Suitable promoters, for example, include but are not limited to CMV, TK, SV40 and EF-1α. In some embodiments, the promoters are inducible, temperature regulated, tissue specific, repressible, heat-shock, developmental, cell lineage specific, eukaryotic, prokaryotic or temporal promoters or a combination or recombination of unmodified or mutagenized, randomized, shuffled sequences of any one or more of the above.

In certain embodiments, vectors disclosed herein comprise a spacer sequence. In one embodiment, the spacer sequence comprises about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 52, 53, 54, 55, 56, 67, 58, 59, 60, 62, 62, 63, 64, 65. 66, 67, 68, 69, 70, 71, 72, 73, 74, 75, 76, 77, 78, 79, 80, between 4 to 630, 152 to 252, 76 to 126, 15 to 35, 36 to 70, 50 to 90, 75 to 125, 30 to 100, 30 to 200, 30 to 500, 30 to 1,000, 100 to 150, 125 to 200, 150 to 300, 200 to 500, 400 to 1,000, 1,000 to 5,000 or greater than 5,000 nucleotides. In one embodiment, the spacer sequence disclosed herein comprises from about 4 to about 630 nucleotides.

In the present disclosure, Chromovert® Technology was used to create cell lines for a diversity of proteins. Cell lines were created both using gene-specific signaling probes or signaling probes directed to plasmid-encoded RNA tags, defined herein as “detection tag”. Detection tags are 3′ untranslated RNA sequence tags subcloned for expression downstream of the stop codon of cDNAs of interest. Unlike epitope tags, detection tags are not translated into protein such that the amino acid sequences of expression products are not altered. Detection tags streamline the cell engineering method by obviating the need to design and develop gene-specific probes. The NaV1.7 (U.S. Pat. No. 9,458,118 issued Oct. 4, 2016), GABA_{A} (US Pat. Publ. No. 2011/0003711), CFTR (US Pat. Publ. No. 2012/0058918) and ENaC (U.S. Pat. No. 9,534,035, issued Jan. 3, 2017) cell lines were all produced using the same detection tags and protocols. Whereas numerous cell-based approaches have been reported to access each of these proteins, the Chromovert® Technology-enabled cell lines and cell-based assays contributes to research and development efforts relating to each of these proteins, as described herein.

In some embodiments, the vectors provided herein comprise a gene of interest selected from the group consisting of Activating Transcription Factor (AFT), Activating Transcription Factor 6 (ATF6), Activating Transcription Factor 6 Alpha (ATF6-Alpha), Activating Transcription Factor 6 Beta (ATF6B), Endoplasmic Reticulum To Nucleus Signaling 1 (IRE1), Endoplasmic Reticulum To Nucleus Signaling 1 Alpha (IRE1 Alpha), Endoplasmic Reticulum To Nucleus Signaling 1 Beta (IRE1 Beta), PERCDC, Activating Transcription Factor 4 (ATF4), YY1 Transcription Factor (YY1), Nuclear Transcription Factor Y Subunit Alpha (NF-YA), Nuclear Transcription Factor Y Subunit Beta (NF-YB), Nuclear Transcription Factor Y Subunit Gamma (NF-YG), X-Box Binding Protein 1 (XBP1), X-Box Binding Protein 1 (XBP2), ER Degradation Enhancing Alpha-Mannosidase Like Protein 1 (EDEM1), ER Degradation Enhancing Alpha-Mannosidase Like Protein 2 (EDEM2), NRF2 Nuclear Factor, Erythroid 2 Like 2 (NRF2), Homocysteine-induced endoplasmic reticulum protein (HERP), X-Linked Inhibitor Of Apoptosis (XIAP), Growth Arrest And DNA Damage-Inducible Protein (GADD34), Peptidylprolyl Isomerase A (PPIA), Peptidylprolyl Isomerase B (PPIB), Peptidylprolyl Isomerase G (PPIG), DnaJ Heat Shock Protein Family (Hsp40) Member C3 (DNAJC3), DnaJ Heat Shock Protein Family (Hsp40) Member C6 (DNAJC6), Positive Regulatory Domain I-Binding Factor 1 (PRDM1), B-Lymphocyte-Induced Maturation Protein 1 (BLIMP1), Calreticulin (CRT, CALR), Calnexin (CNX), Protein Disulfide Isomerase Family A Member 3 (PDIA3), Endoplasmic Reticulum Resident Protein 57 (ERp57), Heat Shock Protein Family A (Hsp70) Member 5 (HSPA5), Binding-Immunoglobulin Protein (BiP), SIL1 Nucleotide Exchange Factor (SIL1), BiP-Associated Protein (BAP), DnaJ Heat Shock Protein Family (Hsp40) Member B 11 (DNAJB11), Endoplasmic Reticulum DNA J Domain-Containing Protein 3 (ERdj3), Calcium Binding Protein 1 (CaBP1), Heat Shock Protein 90 Beta Family Member 1 (HSP90B1), 94 KDa Glucose-Regulated Protein (GRP-94), Protein Disulfide Isomerase Family A Member 4 (PDIA4), Endoplasmic Reticulum Resident Protein 72 (ERp72), Cyclophilin B, Stromal Cell Derived Factor 2 (SDF2), Stromal Cell Derived Factor 2 Like 1 (SDF2L1), Endoplasmic Reticulum Oxidoreductase 1 Alpha (ERO1A), Endoplasmic Reticulum Oxidoreductase 1 Beta (ERO1B), Endoplasmic Reticulum-Associated Degradation (ERAD) pathway gene, Endoplasmic reticulum mannosyl-oligosaccharide 1,2-alpha-mannosidase (MAN1B1), Mannosidase 1, Synoviolin 1 (SYN1), HMG-CoA Reductase Degradation 1 Homolog (HRD1), Stanniocalcin 1 (STC1), Stanniocalcin 1 (STC2), Sarcoplasmic/Endoplasmic Reticulum Calcium ATPase 1 (SERCA1), Sarcoplasmic/Endoplasmic Reticulum Calcium ATPase 1 (SERCA2), Retinitis Pigmentosa GTPase Regulator (RPGR), Cone Dystrophy 1 (COD1), Inositol-3-Phosphate Synthase 1 (ISYNA1), INO1, Sterol Regulatory Element Binding (SREB), Sterol Regulatory Element Binding Transcription Factor 1 (SREBP1), Sterol Regulatory Element Binding Transcription Factor 2 (SREBP2), SREBP1DC, SREBP2DC, Pyruvic Carboxylase (PC), SEC61 Translocon Subunit Alpha 1 (Sec61A1), SEC61 Translocon Subunit Alpha 2 (Sec61A2), SEC61 Translocon Subunit Beta (Sec61B), SEC61 Translocon Subunit Gamma (Sec61G), BCL2 Apoptosis Regulator (BCL2), BCL2 Like 1 (BCL2L1), Bcl-2sp, Bcl-2XL, BCL2 Like 11 (BCL2L11), Bcl-2 Interacting Protein (Bim), X-Ray Repair Cross Complementing 6 (XRCC6), Ku Autoantigen, 70 kDa (Ku70), Voltage Dependent Anion Channel 2 (VDAC2), B Cell Receptor Associated Protein 31 (BCAP31), BCR-Associated Protein 31 (BAP31), Tyrosine 3-Monooxygenase/Tryptophan 5-Monooxygenase Activation Protein Epsilon (YWHAE), and 14-3-3 Protein Epsilon (14-3-3).

In some embodiments, the vectors provided herein comprises a gene of interest as set forth in table 1.

TABLE 1
Exemplary genes of interest

Activating Transcription Factor (AFT)
Activating Transcription Factor 6 (ATF6)
Activating Transcription Factor 6 Alpha (ATF6-Alpha)
Activating Transcription Factor 6 Beta (ATF6B)
Endoplasmic Reticulum To Nucleus Signaling 1 (IRE1)
Endoplasmic Reticulum To Nucleus Signaling 1 Alpha (IRE1 Alpha)
Endoplasmic Reticulum To Nucleus Signaling 1 Beta (IRE1 Beta)
PERCDC
Activating Transcription Factor 4 (ATF4)
YY1 Transcription Factor (YY1)
Nuclear Transcription Factor Y Subunit Alpha (NF-YA)
Nuclear Transcription Factor Y Subunit Beta (NF-YB)
Nuclear Transcription Factor Y Subunit Gamma (NF-YG)
X-Box Binding Protein 1 (XBP1)
X-Box Binding Protein 1 (XBP2)
ER Degradation Enhancing Alpha-Mannosidase Like Protein 1 (EDEM1)
ER Degradation Enhancing Alpha-Mannosidase Like Protein 2 (EDEM2)
NRF2 Nuclear Factor, Erythroid 2 Like 2 (NRF2)
Homocysteine-induced endoplasmic reticulum protein (HERP)
X-Linked Inhibitor Of Apoptosis (XIAP)
Growth Arrest And DNA Damage-Inducible Protein (GADD34)
Peptidylprolyl Isomerase A (PPIA)
Peptidylprolyl Isomerase B (PPIB)
Peptidylprolyl Isomerase G (PPIG)
DnaJ Heat Shock Protein Family (Hsp40) Member C3 (DNAJC3)
DnaJ Heat Shock Protein Family (Hsp40) Member C6 (DNAJC6)
Positive Regulatory Domain I-Binding Factor 1 (PRDM1)
B-Lymphocyte-Induced Maturation Protein 1 (BLIMP1)
Calreticulin (CRT, CALR)
Calnexin (CNX)
Protein Disulfide Isomerase Family A Member 3 (PDIA3)
Endoplasmic Reticulum Resident Protein 57 (ERp57)
Heat Shock Protein Family A (Hsp70) Member 5 (HSPA5)
Binding-Immunoglobulin Protein (BiP)
SIL1 Nucleotide Exchange Factor (SIL1)
BiP-Associated Protein (BAP)
DnaJ Heat Shock Protein Family (Hsp40) Member B11 (DNAJB11)
Endoplasmic Reticulum DNA J Domain-Containing Protein 3 (ERdj3)
Calcium Binding Protein 1 (CaBP1)
Heat Shock Protein 90 Beta Family Member 1 (HSP90B1)
94 KDa Glucose-Regulated Protein (GRP-94)
Protein Disulfide Isomerase Family A Member 4 (PDIA4)
Endoplasmic Reticulum Resident Protein 72 (ERp72)
Cyclophilin B
Stromal Cell Derived Factor 2 (SDF2)
Stromal Cell Derived Factor 2 Like 1 (SDF2L1)
Endoplasmic Reticulum Oxidoreductase 1 Alpha (ERO1A)
Endoplasmic Reticulum Oxidoreductase 1 Beta (ERO1B)
Endoplasmic Reticulum-Associated Degradation (ERAD) pathway gene
Endoplasmic reticulum mannosyl-oligosaccharide 1,2-alpha-mannosidase
(MAN1B1)
Mannosidase 1
Synoviolin 1 (SYN1)
HMG-CoA Reductase Degradation 1 Homolog (HRD1)
Stanniocalcin 1 (STC1)
Stanniocalcin 1 (STC2)
Sarcoplasmic/Endoplasmic Reticulum Calcium ATPase 1 (SERCA1)
Sarcoplasmic/Endoplasmic Reticulum Calcium ATPase 1 (SERCA2)
Retinitis Pigmentosa GTPase Regulator (RPGR)
Cone Dystrophy 1 (COD1)
Inositol-3-Phosphate Synthase 1 (ISYNA1)
INO1
Sterol Regulatory Element Binding (SREB)
Sterol Regulatory Element Binding Transcription Factor 1 (SREBP1)
Sterol Regulatory Element Binding Transcription Factor 2 (SREBP2)
SREBP1DC
SREBP2DC
Pyruvic Carboxylase (PC)
SEC61 Translocon Subunit Alpha 1 (Sec61A1)
SEC61 Translocon Subunit Alpha 2 (Sec61A2)
SEC61 Translocon Subunit Beta (Sec61B)
SEC61 Translocon Subunit Gamma (Sec61G)
BCL2 Apoptosis Regulator (BCL2)
BCL2 Like 1 (BCL2L1)
Bcl-2sp
Bcl-2XL
BCL2 Like 11 (BCL2L11)
Bcl-2 Interacting Protein (Bim)
X-Ray Repair Cross Complementing 6 (XRCC6)
Ku Autoantigen, 70 kDa (Ku70)
Voltage Dependent Anion Channel 2 (VDAC2)
B Cell Receptor Associated Protein 31 (BCAP31)
BCR-Associated Protein 31 (BAP31)
Tyrosine 3-Monooxygenase/Tryptophan 5-Monooxygenase Activation
Protein Epsilon (YWHAE)
14-3-3 Protein Epsilon (14-3-3)

Importantly, the example results provided herein using the methods of the present disclosure demonstrate that the present disclosure constitutes a platform technology that may be applied to produce a cell comprising and/or expressing any sequence of interest. For example, the diversity of proteins (e.g., ENaC, NAV, GABAA, CFTR) include multi-subunit and cytotoxic proteins some of which that had previously remained out-of-reach using cell lines. The methods disclosed in the current application that were used to relied on the use of signaling probes that can hybridize to the same detection tag sequences and without reliance on any gene or protein specific sequence. Similar to how the PCR method may be applied to any nucleic acid sequence without regard to class, type or function, the method disclosed in the current application relies on steps, protocols and principles that are broadly-applicable to any sequence of interest, regardless of class, type or function, for instance including a) exposure of a mixed population of cells to oligonucleotide signaling probes using common transfection reagents known for use to introduce nucleic acids into a wide variety of cell types, b) nucleic acid hybridization of the signaling probes to the detection tag sequences (and not to gene-specific sequences), c) fluorescence-resonance energy transfer (FRET) between the fluorophore and quencher of the signaling probes, d) fluorescence activated cell sorting and e) commonly used tests and assays for the characterization of final cell lines.

The plasmids, vectors, spacers, detection tags and methods of the present application of the present application are contemplated for use to produce cells that express any one or more sequence of interest, regardless of function, type or class. In the case of each example result, commonly used protocols for cell-based assays were the only gene- or sequence specific step used to characterize the cells to select final cell lines. Those with skill in the art can readily choose the relevant test or assay to identify final cell lines based on the function, type or class of the sequence of interested used to produce cell lines, including assays to measure the levels and quality of expression products, including fluorescent cell-based assays. Additional examples of cells that may be produced according to the present disclosure include cells that comprise and/or express sequences of interest that encode proteins including drug targets, membrane proteins, multiunit proteins, cytotoxic proteins, biologics and virus particles. Therefore, the present disclosure includes materials and methods for the preparation of cells expressing any one or more sequences of interest.

NaV1.7 inhibitors are highly sought as non-addictive pain blockers for use to help address the opioid epidemic however stable cell lines comprising multiple accessory subunits have not been reported. The NAV ion channel family is comprised of a large pore-forming a-subunits and up to four accessory β-subunits, p31, p2, p3 and p4, which modulate its activity (O'Malley H A, Isom L L (2015) Sodium channel β subunits: emerging targets in channelopathies, Annu Rev Physiol 77:481-504). HTS of the Chromovert technology-enabled NaV1.7-αβ1β2 cell line resulted in the discovery and development of NaV1.7 inhibitor (U.S. Pat. No. 9,458,118 issued Oct. 4, 2016), a clinical lead compound.

Results using the Chromovert® Technology-enabled panel of four GABA_{A} cell lines provide proof-of-concept for the production of more comprehensive combinatorial panels of GABA_{A} subunits for use to increase understanding of subunit combinations that underlie in vivo function and drug pharmacology. The GABA_{A} ion channel family includes at least 19 different subunits (6 alpha, 3 beta, 3 gamma, 1 delta, 1 epsilon, 1 theta, 1 pi, and 3 rho subunits) thought to form pentameric ion channels comprising 2 alpha subunits, 2 beta subunits and a third subunit (Olsen R W, Sieghart W (2008) International Union of Pharmacology LXX subtypes of y Aminobutyric Acid A receptors: classification on the basis of subunit composition, pharmacology, and function, Update Pharmacol Rev Vol. 60:243-60). Drugs, including approved, failed and candidates drug molecules, may be profiled against such panels to identify in vitro correlates for desired pharmacology and unwanted side-effects.

Chromovert® Technology-enabled CFTR and CFTR-A508 cell lines suggest certain advantages compared to traditional cell-based assays. For instance, whereas transient transfection is generally regarded to produce more robust cell-based assay results as compared to the cell lines, Chromovert® Technology-enabled cell lines with 2 to 6 fold or more functionality were isolated. Also, no CFTR-A508 cell lines have been reported to result in cell-based assay for the detection of CFTR-A508 in the absence of trafficking correctors used to release the mutant CFTR trapped in intracellular compartments. In contrast, the Chromovert® Technology-enabled CFTR-A508 cell line resulted in a detectable cell-based assay signal in the absence of any trafficking correctors. This CFTR mutant is the most prevalent in Cystic Fibrosis. The creation of a cell-based for CFTR-A508 that does not necessitate the use of trafficking correctors may provide for more physiologically relevant drug discovery.

Finally, ENaC is a highly-sought heteromultimeric ion channel drug target comprising a, b and g subunits expressed on the apical surface of epithelial cells where it conducts sodium and plays a role in fluid balance (Fornius 2013). In the lung, ENaC is a validated drug target in pulmonary edema (PE), Chronic Obstructive Pulmonary Disease (COPD) and Cystic Fibrosis (CF), with potential to treat COVID-19 (Matalon 2015). ENaC has also been postulated as the putative human salt taste receptor (Roper 2015), where salt taste enhancers could be used to reduce dietary sodium. However, cell-based drug discovery against this validated drug target has been hampered due to its cytotoxicity in cultured cells. Only Chromovert® Technology enabled the creation of an αβγ-ENaC cell line. The creation of the ENaC cell line in turn enabled limiting proteolysis to achieve functionally distinct proteolyzed variants, resulting in the discovery of an ENaC potentiator and human sensory confirmation of ENaC's role as a human salt taste receptor.

Many cultured cells exhibit vast cell-to-cell genetic diversity. For example, according to the product specifications at ATCC, the HEK 293 cell line is characterized as follows: “This is a hypotriploid human cell line. The modal chromosome number was 64, occurring in 30% of cells. The rate of cells with higher ploidies was 4.2%.” It is possible that the signaling probes used in Chromovert® Technology may serve as biomarkers to detect and isolate those cells that are compatible with the viable and functional expression of biological targets that are otherwise cytotoxic in cell cultures.

Procedures and protocols required for gene subcloning, transformation, maintenance and growth of bacterial cultures will be performed by methods well known in the art. Standard molecular cloning techniques are described by Sambrook, J., Fritsch, E. F. and Maniatis, T. (Molecular Cloning: A Laboratory Manual, 2nd ed.; Cold Spring Harbor Laboratory: Cold Spring Harbor, New York, 1989). Manipulations with bacteria are described by Phillipp Gerhardt, R. G. E. Murray, Ralph N. Costilow, Eugene W. Nester, Willis A. Wood, Noel R. Krieg and G. Briggs Phillips (Methods for General Bacteriology; American Society for Microbiology: Washington, D C, 1994).

TABLE 2
Sequences

| SEQ | | |
|---|---|---|
| ID NO | Description | Sequence |
| 1 | plasmid | atgcattagttattaatagtaatcaattacggggtcattagttcatagcccatatatggagttccgcgttaca |
| | pCP4-N | taacttacggtaaatggcccgcctggctgaccgcccaacgacccccgcccattgacgtcaataatgac |
| | | gtatgttcccatagtaacgccaatagggactttccattgacgtcaatgggggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaccgg |
| | | tggtaccaggtaagtgtacccaattcgccctatagtgagtcgtattacaattcactcgatcgcccttccca |
| | | acagttgcgcagcctgaatggcgaatggagatccaatttttaagtgtataatgtgttaaactactgattcta |
| | | attgtttgtgtattttagattcacagtcccaaggctcatttcaggcccctcagtcctcacagtctgttcatgat |
| | | cataatcagccataccacatttgtagaggttttacttgctttaaaaaacctcccacacctccccctgaacct |
| | | gaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgt |
| | | atcttaacgcgtttaccaatgcttaatcagtgaggcacctatctcagcgatctgtctatttcgttcatccata |
| | | gttgcctgactccccgtcgtgtagataactacgatacgggagggcttaccatctggccccagtgctgca |
| | | atgataccgcgagacccacgctcaccggctccagatttatcagcaataaaccagccagccggaaggg |
| | | ccgagcgcagaagtggtcctgcaactttatccgcctccatccagtctattaattgttgccgggaagctag |
| | | agtaagtagttcgccagttaatagtttgcgcaacgttgttgccattgctacaggcatcgtggtgtcacgct |
| | | cgtcgtttggtatggcttcattcagctccggttcccaacgatcaaggcgagttacatgatcccccatgttg |
| | | tgcaaaaaagcggttagctccttcggtcctccgatcgttgtcagaagtaagttggccgcagtgttatcac |
| | | tcatggttatggcagcactgcataattctcttactgtcatgccatccgtaagatgcttttctgtgactggtga |
| | | gtactcaaccaagtcattctgagaatagtgtatgcggcgaccgagttgctcttgcccggcgtcaatacg |
| | | ggataataccgcgccacatagcagaactttaaaagtgctcatcattggaaaacgttcttcggggcgaaa |
| | | actctcaaggatcttaccgctgttgagatccagttcgatgtaacccactcgtgcacccaactgatcttcag |
| | | catcttttactttcaccagcgtttctgggtgagcaaaaacaggaaggcaaaatgccgcaaaaaaggga |
| | | ataagggcgacacggaaatgttgaatactcatactcttcctttttcaatattattgaagcatttatcagggtt |
| | | attgtctcatgagcggatacatatttgaatgtatttagaaaaataaacaaataggggtacgcgtaaattgta |
| | | agcgttaatattttgttaaaattcgcgttaaatttttgttaaatcagctcattttttaaccaataggccgaaatc |
| | | ggcaaaatcccttataaatcaaaagaatagaccgagatagggttgagtgttgttccagtttggaacaag |
| | | agtccactattaaagaacgtggactccaacgtcaaagggcgaaaaaccgtctatcagggcgatggcc |
| | | cactacgtgaaccatcaccctaatcaagttttttggggtcgaggtgccgtaaagcactaaatcggaacc |
| | | ctaaagggagcccccgatttagagcttgacggggaaagccggcgaacgtggcgagaaaggaaggg |
| | | aagaaagcgaaaggagcgggcgctagggcgctggcaagtgtagcggtcacgctgcgcgtaaccac |
| | | cacacccgccgcgcttaatgcgccgctacagggcgcgtcaggtggcacttttcggggaaatgtgcgc |
| | | ggaacccctatttgtttatttttctaaatacattcaaatatgtatccgctcatgagacaataaccctgataaat |
| | | gcttcaataatattgaaaaaggaagaatcctgaggcggaaagaaccagctgtggaatgtgtgtcagtta |
| | | gggtgtggaaagtccccaggctccccagcaggcagaagtatgcaaagcatgcatctcaattagtcag |
| | | caaccaggtgtggaaagtccccaggctccccagcaggcagaagtatgcaaagcatgcatctcaatta |
| | | gtcagcaaccatagtcccgcccctaactccgcccatcccgcccctaactccgcccagttccgcccattc |
| | | tccgccccatggctgactaattttttttatttatgcagaggccgaggccgcctcggcctctgagctattcc |
| | | agaagtagtgaggaggcttttttggaggcctaggcttttgcaaatatcgattaagagacaggatgagga |
| | | tcgtttcgcatgattgaacaagatggattgcacgcaggttctccggccgcttgggtggagaggctattc |
| | | ggctatgactgggcacaacagacaatcggctgctctgatgccgccgtgttccggctgtcagcgcagg |
| | | ggcgcccggttctttttgtcaagaccgacctgtccggtgccctgaatgaactgcaagacgaggcagcg |
| | | cggctatcgtggctggccacgacgggcgttccttgcgcagctgtgctcgacgttgtcactgaagcggg |
| | | aagggactggctgctattgggcgaagtgccggggcaggatctcctgtcatctcaccttgctcctgccg |
| | | agaaagtatccatcatggctgatgcaatgcggcggctgcatacgcttgatccggctacctgcccattcg |
| | | accaccaagcgaaacatcgcatcgagcgagcacgtactcggatggaagccggtcttgtcgatcagga |
| | | tgatctggacgaagaacatcaggggctcgcgccagccgaactgttcgccaggctcaaggcgagcat |
| | | gcccgacggcgaggatctcgtcgtgacccatggcgatgcctgcttgccgaatatcatggtggaaaatg |
| | | gccgcttttctggattcatcgactgtggccggctgggtgtggcggaccgctatcaggacatagcgttgg |
| | | ctacccgtgatattgctgaagaacttggcggcgaatgggctgaccgcttcctcgtgctttacggtatcgc |
| | | cgctcccgattcgcagcgcatcgccttctatcgccttcttgacgagttcttctgagcgggactctggggtt |
| | | cgaaatgaccgaccaagcgacgcccaacctgccatcacgagatttcgattccaccgccgccttctatg |
| | | aaaggttgggcttcggaatcgttttccgggacgccggctggatgatcctccagcgcggggatctcatg |
| | | ctggagttcttcgcccaccctagggggaggctaactgaaacacggaaggagacaataccggaagga |
| | | acccgcgctatgacggcaataaaaagacagaataaaacgcacggtgttgggtcgtttgttcataaacg |
| | | cggggttcggtcccagggctggcactctgtcgataccccaccgagaccccattggggccaatacgcc |
| | | cgcgtttcttccttttccccaccccaccccccaagttcgggtgaaggcccagggctcgcagccaacgtc |
| | | ggggcggcaggccctgccatagcctcaggttactcatatatactttagattgatttaaaacttcatttttaat |
| | | ttaaaaggatctaggtgaagatcctttttgataatctcatgaccaaaatcccttaacgtgagttttcgttcca |
| | | ctgagcgtcagaccccgtagaaaagatcaaaggatcttcttgagatcctttttttctgcgcgtaatctgct |
| | | gcttgcaaacaaaaaaaccaccgctaccagcggtggtttgtttgccggatcaagagctaccaactctttt |
| | | tccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgct |
| | | gccagtggcgataagtcgtgtcttaccgggttggactcaagacgatagttaccggataaggcgcagcg |
| | | gtcgggctgaacggggggttcgtgcacacagcccagcttggagcgaacgacctacaccgaactgag |
| | | atacctacagcgtgagctatgagaaagcgccacgcttcccgaagggagaaaggcggacaggtatcc |
| | | ggtaagcggcagggtcggaacaggagagcgcacgagggagcttccagggggaaacgcctggtat |
| | | ctttatagtcctgtcgggtttcgccacctctgacttgagcgtcgatttttgtgatgctcgtcaggggggcg |
| | | gagcctatggaaaaacgccagcaacgcggcctttttacggttcctggccttttgctggccttttgctcaca |
| | | tgttctttcctgcgttatcccctgattctgtggataaccgtattaccgcc |
| 2 | plasmid | atgcattagttattaatagtaatcaattacggggtcattagttcatagcccatatatggagttccgcgttaca |
| | pCP4-N1 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggaggcaggtggacaggaaggttc |
| | | taatgttcttaaggcacaggaactgggacatctgggcccggaaagcctttttctctgtgatccggtacag |
| | | tccttctgcggtaccaggtaagtgtacccaattcgccctatagtgagtcgtattacaattcactcgatcgc |
| | | ccttcccaacagttgcgcagcctgaatggcgaatggagatccaatttttaagtgtataatgtgttaaacta |
| | | ctgattctaattgtttgtgtattttagattcacagtcccaaggctcatttcaggcccctcagtcctcacagtct |
| | | gttcatgatcataatcagccataccacatttgtagaggttttacttgctttaaaaaacctcccacacctccc |
| | | cctgaacctgaaacataaaatgaatgcaattgttgttgttaacttgtttattgcagcttataatggttacaaat |
| | | aaagcaatagcatcacaaatttcacaaataaagcatttttttcactgcattctagttgtggtttgtccaaact |
| | | catcaatgtatcttaacgcgtttaccaatgcttaatcagtgaggcacctatctcagcgatctgtctatttcgt |
| | | tcatccatagttgcctgactccccgtcgtgtagataactacgatacgggagggcttaccatctggcccca |
| | | gtgctgcaatgataccgcgagacccacgctcaccggctccagatttatcagcaataaaccagccagcc |
| | | ggaagggccgagcgcagaagtggtcctgcaactttatccgcctccatccagtctattaattgttgccgg |
| | | gaagctagagtaagtagttcgccagttaatagtttgcgcaacgttgttgccattgctacaggcatcgtggt |
| | | gtcacgctcgtcgtttggtatggcttcattcagctccggttcccaacgatcaaggcgagttacatgatccc |
| | | ccatgttgtgcaaaaaagcggttagctccttcggtcctccgatcgttgtcagaagtaagttggccgcagt |
| | | gttatcactcatggttatggcagcactgcataattctcttactgtcatgccatccgtaagatgcttttctgtga |
| | | ctggtgagtactcaaccaagtcattctgagaatagtgtatgcggcgaccgagttgctcttgcccggcgt |
| | | caatacgggataataccgcgccacatagcagaactttaaaagtgctcatcattggaaaacgttcttcgg |
| | | ggcgaaaactctcaaggatcttaccgctgttgagatccagttcgatgtaacccactcgtgcacccaact |
| | | gatcttcagcatcttttactttcaccagcgtttctgggtgagcaaaaacaggaaggcaaaatgccgcaaa |
| | | aaagggaataagggcgacacggaaatgttgaatactcatactcttcctttttcaatattattgaagcatttat |
| | | cagggttattgtctcatgagcggatacatatttgaatgtatttagaaaaataaacaaataggggtacgcgt |
| | | aaattgtaagcgttaatattttgttaaaattcgcgttaaatttttgttaaatcagctcattttttaaccaataggc |
| | | cgaaatcggcaaaatcccttataaatcaaaagaatagaccgagatagggttgagtgttgttccagtttgg |
| | | aacaagagtccactattaaagaacgtggactccaacgtcaaagggcgaaaaaccgtctatcagggcg |
| | | atggcccactacgtgaaccatcaccctaatcaagttttttggggtcgaggtgccgtaaagcactaaatcg |
| | | gaaccctaaagggagcccccgatttagagcttgacggggaaagccggcgaacgtggcgagaaagg |
| | | aagggaagaaagcgaaaggagcgggcgctagggcgctggcaagtgtagcggtcacgctgcgcgt |
| | | aaccaccacacccgccgcgcttaatgcgccgctacagggcgcgtcaggtggcacttttcggggaaat |
| | | gtgcgcggaacccctatttgtttatttttctaaatacattcaaatatgtatccgctcatgagacaataaccct |
| | | gataaatgcttcaataatattgaaaaaggaagaatcctgaggcggaaagaaccagctgtggaatgtgt |
| | | gtcagttagggtgtggaaagtccccaggctccccagcaggcagaagtatgcaaagcatgcatctcaat |
| | | tagtcagcaaccaggtgtggaaagtccccaggctccccagcaggcagaagtatgcaaagcatgcatc |
| | | tcaattagtcagcaaccatagtcccgcccctaactccgcccatcccgcccctaactccgcccagttccg |
| | | cccattctccgccccatggctgactaattttttttatttatgcagaggccgaggccgcctcggcctctgag |
| | | ctattccagaagtagtgaggaggcttttttggaggcctaggcttttgcaaatatcgattaagagacaggat |
| | | gaggatcgtttcgcatgattgaacaagatggattgcacgcaggttctccggccgcttgggtggagagg |
| | | ctattcggctatgactgggcacaacagacaatcggctgctctgatgccgccgtgttccggctgtcagcg |
| | | caggggcgcccggttctttttgtcaagaccgacctgtccggtgccctgaatgaactgcaagacgaggc |
| | | agcgcggctatcgtggctggccacgacgggcgttccttgcgcagctgtgctcgacgttgtcactgaag |
| | | cgggaagggactggctgctattgggcgaagtgccggggcaggatctcctgtcatctcaccttgctcct |
| | | gccgagaaagtatccatcatggctgatgcaatgcggggctgcatacgcttgatccggctacctgccc |
| | | attcgaccaccaagcgaaacatcgcatcgagcgagcacgtactcggatggaagccggtcttgtcgat |
| | | caggatgatctggacgaagaacatcaggggctcgcgccagccgaactgttcgccaggctcaaggcg |
| | | agcatgcccgacggcgaggatctcgtcgtgacccatggcgatgcctgcttgccgaatatcatggtgga |
| | | aaatggccgcttttctggattcatcgactgtggccggctgggtgtggcggaccgctatcaggacatagc |
| | | gttggctacccgtgatattgctgaagaacttggcggcgaatgggctgaccgcttcctcgtgctttacggt |
| | | atcgccgctcccgattcgcagcgcatcgccttctatcgccttcttgacgagttcttctgagcgggactctg |
| | | gggttcgaaatgaccgaccaagcgacgcccaacctgccatcacgagatttcgattccaccgccgcctt |
| | | ctatgaaaggttgggcttcggaatcgttttccgggacgccggctggatgatcctccagcgcggggatct |
| | | catgctggagttcttcgcccaccctagggggaggctaactgaaacacggaaggagacaataccgga |
| | | aggaacccgcgctatgacggcaataaaaagacagaataaaacgcacggtgttgggtcgtttgttcata |
| | | aacgcggggttcggtcccagggctggcactctgtcgataccccaccgagaccccattggggccaata |
| | | cgcccgcgtttcttccttttccccaccccaccccccaagttcgggtgaaggcccagggctcgcagcca |
| | | acgtcggggcggcaggccctgccatagcctcaggttactcatatatactttagattgatttaaaacttcatt |
| | | tttaatttaaaaggatctaggtgaagatcctttttgataatctcatgaccaaaatcccttaacgtgagttttcg |
| | | ttccactgagcgtcagaccccgtagaaaagatcaaaggatcttcttgagatcctttttttctgcgcgtaatc |
| | | tgctgcttgcaaacaaaaaaaccaccgctaccagcggtggtttgtttgccggatcaagagctaccaact |
| | | ctttttccgaaggtaactggcttcagcagagcgcagataccaaatactgtccttctagtgtagccgtagtt |
| | | aggccaccacttcaagaactctgtagcaccgcctacatacctcgctctgctaatcctgttaccagtggct |
| | | gctgccagtggcgataagtcgtgtcttaccgggttggactcaagacgatagttaccggataaggcgca |
| | | gcggtcgggctgaacggggggttcgtgcacacagcccagcttggagcgaacgacctacaccgaact |
| | | gagatacctacagcgtgagctatgagaaagcgccacgcttcccgaagggagaaaggcggacaggt |
| | | atccggtaagcggcagggtcggaacaggagagcgcacgagggagcttccagggggaaacgcctg |
| | | gtatctttatagtcctgtcgggtttcgccacctctgacttgagcgtcgatttttgtgatgctcgtcaggggg |
| | | gcggagcctatggaaaaacgccagcaacgcggcctttttacggttcctggccttttgctggccttttgct |
| | | cacatgttctttcctgcgttatcccctgattctgtggataaccgtattaccgcc |
| 3 | plasmid | atgcattagttattaatagtaatcaattacggggtcattagttcatagcccatatatggagttccgcgttaca |
| | pCP4-N2 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggtaccaagcttcgaggcaggtgg |
| | | acagcttggttctaatgaagttaaccctgtcgttctgcgacatctgggcccggaaagcgtttaactgatg |
| | | gatggaacagtccttctgcggtaccaggtaagtgtacccaattcgccctatagtgagtcgtattacaattc |
| | | actcgatcgcccttcccaacagttgcgcagcctgaatggcgaatggagatccaatttttaagtgtataat |
| | | gtgttaaactactgattctaattgtttgtgtattttagattcacagtcccaaggctcatttcaggcccctcagt |
| | | cctcacagtctgttcatgatcataatcagccataccacatttgtagaggttttacttgctttaaaaaacctcc |
| | | cacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtt |
| | | tgtccaaactcatcaatgtatcttaacgcgtttaccaatgcttaatcagtgaggcacctatctcagcgatct |
| | | gtctatttcgttcatccatagttgcctgactccccgtcgtgtagataactacgatacgggagggcttacca |
| | | tctggccccagtgctgcaatgataccgcgagacccacgctcaccggctccagatttatcagcaataaa |
| | | ccagccagccggaagggccgagcgcagaagtggtcctgcaactttatccgcctccatccagtctatta |
| | | attgttgccgggaagctagagtaagtagttcgccagttaatagtttgcgcaacgttgttgccattgctaca |
| | | ggcatcgtggtgtcacgctcgtcgtttggtatggcttcattcagctccggttcccaacgatcaaggcgag |
| | | ttacatgatcccccatgttgtgcaaaaaagcggttagctccttcggtcctccgatcgttgtcagaagtaag |
| | | ttggccgcagtgttatcactcatggttatggcagcactgcataattctcttactgtcatgccatccgtaaga |
| | | tgcttttctgtgactggtgagtactcaaccaagtcattctgagaatagtgtatgcggcgaccgagttgctc |
| | | ttgcccggcgtcaatacgggataataccgcgccacatagcagaactttaaaagtgctcatcattggaaa |
| | | acgttcttcggggcgaaaactctcaaggatcttaccgctgttgagatccagttcgatgtaacccactcgt |
| | | gcacccaactgatcttcagcatcttttactttcaccagcgtttctgggtgagcaaaaacaggaaggcaaa |
| | | atgccgcaaaaaagggaataagggcgacacggaaatgttgaatactcatactcttcctttttcaatattat |
| | | tgaagcatttatcagggttattgtctcatgagcggatacatatttgaatgtatttagaaaaataaacaaata |
| | | ggggtacgcgtaaattgtaagcgttaatattttgttaaaattcgcgttaaatttttgttaaatcagctcatttttt |
| | | aaccaataggccgaaatcggcaaaatcccttataaatcaaaagaatagaccgagatagggttgagtgtt |
| | | gttccagtttggaacaagagtccactattaaagaacgtggactccaacgtcaaagggcgaaaaaccgt |
| | | ctatcagggcgatggcccactacgtgaaccatcaccctaatcaagttttttggggtcgaggtgccgtaa |
| | | agcactaaatcggaaccctaaagggagcccccgatttagagcttgacggggaaagccggcgaacgt |
| | | ggcgagaaaggaagggaagaaagcgaaaggagcgggcgctagggcgctggcaagtgtagcggt |
| | | cacgctgcgcgtaaccaccacacccgccgcgcttaatgcgccgctacagggcgcgtcaggtggcac |
| | | ttttcggggaaatgtgcgcggaacccctatttgtttatttttctaaatacattcaaatatgtatccgctcatga |
| | | gacaataaccctgataaatgcttcaataatattgaaaaaggaagaatcctgaggcggaaagaaccagc |
| | | tgtggaatgtgtgtcagttagggtgtggaaagtccccaggctccccagcaggcagaagtatgcaaagc |
| | | atgcatctcaattagtcagcaaccaggtgtggaaagtccccaggctccccagcaggcagaagtatgca |
| | | aagcatgcatctcaattagtcagcaaccatagtcccgcccctaactccgcccatcccgcccctaactcc |
| | | gcccagttccgcccattctccgccccatggctgactaattttttttatttatgcagaggccgaggccgcct |
| | | cggcctctgagctattccagaagtagtgaggaggcttttttggaggcctaggcttttgcaaatatcgatta |
| | | agagacaggatgaggatcgtttcgcatgattgaacaagatggattgcacgcaggttctccggccgctt |
| | | gggtggagaggctattcggctatgactgggcacaacagacaatcggctgctctgatgccgccgtgttc |
| | | cggctgtcagcgcaggggcgcccggttctttttgtcaagaccgacctgtccggtgccctgaatgaact |
| | | gcaagacgaggcagcgcggctatcgtggctggccacgacgggcgttccttgcgcagctgtgctcga |
| | | cgttgtcactgaagcgggaagggactggctgctattgggcgaagtgccggggcaggatctcctgtcat |
| | | ctcaccttgctcctgccgagaaagtatccatcatggctgatgcaatgcggcggctgcatacgcttgatc |
| | | cggctacctgcccattcgaccaccaagcgaaacatcgcatcgagcgagcacgtactcggatggaag |
| | | ccggtcttgtcgatcaggatgatctggacgaagaacatcaggggctcgcgccagccgaactgttcgc |
| | | caggctcaaggcgagcatgcccgacggcgaggatctcgtcgtgacccatggcgatgcctgcttgcc |
| | | gaatatcatggtggaaaatggccgcttttctggattcatcgactgtggccggctgggtgtggcggaccg |
| | | ctatcaggacatagcgttggctacccgtgatattgctgaagaacttggcggcgaatgggctgaccgctt |
| | | cctcgtgctttacggtatcgccgctcccgattcgcagcgcatcgccttctatcgccttcttgacgagttctt |
| | | ctgagcgggactctggggttcgaaatgaccgaccaagcgacgcccaacctgccatcacgagatttcg |
| | | attccaccgccgccttctatgaaaggttgggcttcggaatcgttttccgggacgccggctggatgatcct |
| | | ccagcgcggggatctcatgctggagttcttcgcccaccctagggggaggctaactgaaacacggaag |
| | | gagacaataccggaaggaacccgcgctatgacggcaataaaaagacagaataaaacgcacggtgtt |
| | | gggtcgtttgttcataaacgcggggttcggtcccagggctggcactctgtcgataccccaccgagacc |
| | | ccattggggccaatacgcccgcgtttcttccttttccccaccccaccccccaagttcgggtgaaggccc |
| | | agggctcgcagccaacgtcggggcggcaggccctgccatagcctcaggttactcatatatactttaga |
| | | ttgatttaaaacttcatttttaatttaaaaggatctaggtgaagatcctttttgataatctcatgaccaaaatcc |
| | | cttaacgtgagttttcgttccactgagcgtcagaccccgtagaaaagatcaaaggatcttcttgagatcct |
| | | ttttttctgcgcgtaatctgctgcttgcaaacaaaaaaaccaccgctaccagcggtggtttgtttgccgga |
| | | tcaagagctaccaactctttttccgaaggtaactggcttcagcagagcgcagataccaaatactgtcctt |
| | | ctagtgtagccgtagttaggccaccacttcaagaactctgtagcaccgcctacatacctcgctctgctaa |
| | | tcctgttaccagtggctgctgccagtggcgataagtcgtgtcttaccgggttggactcaagacgatagtt |
| | | accggataaggcgcagcggtcgggctgaacggggggttcgtgcacacagcccagcttggagcgaa |
| | | cgacctacaccgaactgagatacctacagcgtgagctatgagaaagcgccacgcttcccgaaggga |
| | | gaaaggcggacaggtatccggtaagcggcagggtcggaacaggagagcgcacgagggagcttcc |
| | | agggggaaacgcctggtatctttatagtcctgtcgggtttcgccacctctgacttgagcgtcgatttttgtg |
| | | atgctcgtcaggggggcggagcctatggaaaaacgccagcaacgcggcctttttacggttcctggcct |
| | | tttgctggccttttgctcacatgttctttcctgcgttatcccctgattctgtggataaccgtattaccgcc |
| 4 | plasmid | atgcattagttattaatagtaatcaattacggggtcattagttcatagcccatatatggagttccgcgttaca |
| | pCP4-N3 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaaggg |
| | | cgaattcggatccgcggccgccttaagctcgaggcaggtggacaggaaggttctaatgttctataggg |
| | | tctgcttgtcgctcatctgggcccggagatgcgtaaagtcagacatccggtacagtccttctgcggtacc |
| | | aggtaagtgtacccaattcgccctatagtgagtcgtattacaattcactcgatcgcccttcccaacagttg |
| | | cgcagcctgaatggcgaatggagatccaatttttaagtgtataatgtgttaaactactgattctaattgtttg |
| | | tgtattttagattcacagtcccaaggctcatttcaggcccctcagtcctcacagtctgttcatgatcataatc |
| | | agccataccacatttgtagaggttttacttgctttaaaaaacctcccacacctccccctgaacctgaaaca |
| | | taaaatgaatgcaattgttgttgttaacttgtttattgcagcttataatggttacaaataaagcaatagcatca |
| | | caaatttcacaaataaagcatttttttcactgcattctagttgtggtttgtccaaactcatcaatgtatcttaac |
| | | gcgtttaccaatgcttaatcagtgaggcacctatctcagcgatctgtctatttcgttcatccatagttgcctg |
| | | actccccgtcgtgtagataactacgatacgggagggcttaccatctggccccagtgctgcaatgatacc |
| | | gcgagacccacgctcaccggctccagatttatcagcaataaaccagccagccggaagggccgagcg |
| | | cagaagtggtcctgcaactttatccgcctccatccagtctattaattgttgccgggaagctagagtaagta |
| | | gttcgccagttaatagtttgcgcaacgttgttgccattgctacaggcatcgtggtgtcacgctcgtcgtttg |
| | | gtatggcttcattcagctccggttcccaacgatcaaggcgagttacatgatcccccatgttgtgcaaaaa |
| | | agcggttagctccttcggtcctccgatcgttgtcagaagtaagttggccgcagtgttatcactcatggtta |
| | | tggcagcactgcataattctcttactgtcatgccatccgtaagatgcttttctgtgactggtgagtactcaa |
| | | ccaagtcattctgagaatagtgtatgcggcgaccgagttgctcttgcccggcgtcaatacgggataata |
| | | ccgcgccacatagcagaactttaaaagtgctcatcattggaaaacgttcttcggggcgaaaactctcaa |
| | | ggatcttaccgctgttgagatccagttcgatgtaacccactcgtgcacccaactgatcttcagcatctttta |
| | | ctttcaccagcgtttctgggtgagcaaaaacaggaaggcaaaatgccgcaaaaaagggaataagggc |
| | | gacacggaaatgttgaatactcatactcttcctttttcaatattattgaagcatttatcagggttattgtctcat |
| | | gagcggatacatatttgaatgtatttagaaaaataaacaaataggggtacgcgtaaattgtaagcgttaat |
| | | attttgttaaaattcgcgttaaatttttgttaaatcagctcattttttaaccaataggccgaaatcggcaaaat |
| | | cccttataaatcaaaagaatagaccgagatagggttgagtgttgttccagtttggaacaagagtccacta |
| | | ttaaagaacgtggactccaacgtcaaagggcgaaaaaccgtctatcagggcgatggcccactacgtg |
| | | aaccatcaccctaatcaagttttttggggtcgaggtgccgtaaagcactaaatcggaaccctaaaggga |
| | | gcccccgatttagagcttgacggggaaagccggcgaacgtggcgagaaaggaagggaagaaagc |
| | | gaaaggagcgggcgctagggcgctggcaagtgtagcggtcacgctgcgcgtaaccaccacacccg |
| | | ccgcgcttaatgcgccgctacagggcgcgtcaggtggcacttttcggggaaatgtgcgcggaacccc |
| | | tatttgtttatttttctaaatacattcaaatatgtatccgctcatgagacaataaccctgataaatgcttcaata |
| | | atattgaaaaaggaagaatcctgaggcggaaagaaccagctgtggaatgtgtgtcagttagggtgtgg |
| | | aaagtccccaggctccccagcaggcagaagtatgcaaagcatgcatctcaattagtcagcaaccagg |
| | | tgtggaaagtccccaggctccccagcaggcagaagtatgcaaagcatgcatctcaattagtcagcaac |
| | | catagtcccgcccctaactccgcccatcccgcccctaactccgcccagttccgcccattctccgcccca |
| | | tggctgactaattttttttatttatgcagaggccgaggccgcctcggcctctgagctattccagaagtagt |
| | | gaggaggcttttttggaggcctaggcttttgcaaatatcgattaagagacaggatgaggatcgtttcgca |
| | | tgattgaacaagatggattgcacgcaggttctccggccgcttgggtggagaggctattcggctatgact |
| | | gggcacaacagacaatcggctgctctgatgccgccgtgttccggctgtcagcgcaggggcgcccgg |
| | | ttctttttgtcaagaccgacctgtccggtgccctgaatgaactgcaagacgaggcagcgcggctatcgt |
| | | ggctggccacgacgggcgttccttgcgcagctgtgctcgacgttgtcactgaagcgggaagggactg |
| | | gctgctattgggcgaagtgccggggcaggatctcctgtcatctcaccttgctcctgccgagaaagtatc |
| | | catcatggctgatgcaatgcggcggctgcatacgcttgatccggctacctgcccattcgaccaccaag |
| | | cgaaacatcgcatcgagcgagcacgtactcggatggaagccggtcttgtcgatcaggatgatctgga |
| | | cgaagaacatcaggggctcgcgccagccgaactgttcgccaggctcaaggcgagcatgcccgacg |
| | | gcgaggatctcgtcgtgacccatggcgatgcctgcttgccgaatatcatggtggaaaatggccgctttt |
| | | ctggattcatcgactgtggccggctgggtgtggcggaccgctatcaggacatagcgttggctacccgt |
| | | gatattgctgaagaacttggcggcgaatgggctgaccgcttcctcgtgctttacggtatcgccgctccc |
| | | gattcgcagcgcatcgccttctatcgccttcttgacgagttcttctgagcgggactctggggttcgaaatg |
| | | accgaccaagcgacgcccaacctgccatcacgagatttcgattccaccgccgccttctatgaaaggtt |
| | | gggcttcggaatcgttttccgggacgccggctggatgatcctccagcgcggggatctcatgctggagt |
| | | tcttcgcccaccctagggggaggctaactgaaacacggaaggagacaataccggaaggaacccgc |
| | | gctatgacggcaataaaaagacagaataaaacgcacggtgttgggtcgtttgttcataaacgcggggtt |
| | | cggtcccagggctggcactctgtcgataccccaccgagaccccattggggccaatacgcccgcgttt |
| | | cttccttttccccaccccaccccccaagttcgggtgaaggcccagggctcgcagccaacgtcggggc |
| | | ggcaggccctgccatagcctcaggttactcatatatactttagattgatttaaaacttcatttttaatttaaaa |
| | | ggatctaggtgaagatcctttttgataatctcatgaccaaaatcccttaacgtgagttttcgttccactgag |
| | | cgtcagaccccgtagaaaagatcaaaggatcttcttgagatcctttttttctgcgcgtaatctgctgcttgc |
| | | aaacaaaaaaaccaccgctaccagcggtggtttgtttgccggatcaagagctaccaactctttttccgaa |
| | | ggtaactggcttcagcagagcgcagataccaaatactgtccttctagtgtagccgtagttaggccacca |
| | | cttcaagaactctgtagcaccgcctacatacctcgctctgctaatcctgttaccagtggctgctgccagtg |
| | | gcgataagtcgtgtcttaccgggttggactcaagacgatagttaccggataaggcgcagcggtcggg |
| | | ctgaacggggggttcgtgcacacagcccagcttggagcgaacgacctacaccgaactgagataccta |
| | | cagcgtgagctatgagaaagcgccacgcttcccgaagggagaaaggcggacaggtatccggtaag |
| | | cggcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcctat |
| | | ggaaaaacgccagcaacgcggcctttttacggttcctggccttttgctggccttttgctcacatgttctttc |
| | | ctgcgttatcccctgattctgtggataaccgtattaccgcc |
| 5 | plasmid | atgcattagttattaatagtaatcaattacggggtcattagttcatagcccatatatggagttccgcgttaca |
| | pCP5-P | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaccgg |
| | | tggtaccaggtaagtgtacccaattcgccctatagtgagtcgtattacaattcactcgatcgcccttccca |
| | | acagttgcgcagcctgaatggcgaatggagatccaatttttaagtgtataatgtgttaaactactgattcta |
| | | attgtttgtgtattttagattcacagtcccaaggctcatttcaggcccctcagtcctcacagtctgttcatgat |
| | | cataatcagccataccacatttgtagaggttttacttgctttaaaaaacctcccacacctccccctgaacct |
| | | gaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgt |
| | | atcttaacgcgtttaccaatgcttaatcagtgaggcacctatctcagcgatctgtctatttcgttcatccata |
| | | gttgcctgactccccgtcgtgtagataactacgatacgggagggcttaccatctggccccagtgctgca |
| | | atgataccgcgagacccacgctcaccggctccagatttatcagcaataaaccagccagccggaaggg |
| | | ccgagcgcagaagtggtcctgcaactttatccgcctccatccagtctattaattgttgccgggaagctag |
| | | agtaagtagttcgccagttaatagtttgcgcaacgttgttgccattgctacaggcatcgtggtgtcacgct |
| | | cgtcgtttggtatggcttcattcagctccggttcccaacgatcaaggcgagttacatgatcccccatgttg |
| | | tgcaaaaaagcggttagctccttcggtcctccgatcgttgtcagaagtaagttggccgcagtgttatcac |
| | | tcatggttatggcagcactgcataattctcttactgtcatgccatccgtaagatgcttttctgtgactggtga |
| | | gtactcaaccaagtcattctgagaatagtgtatgcggcgaccgagttgctcttgcccggcgtcaatacg |
| | | ggataataccgcgccacatagcagaactttaaaagtgctcatcattggaaaacgttcttcggggcgaaa |
| | | actctcaaggatcttaccgctgttgagatccagttcgatgtaacccactcgtgcacccaactgatcttcag |
| | | catcttttactttcaccagcgtttctgggtgagcaaaaacaggaaggcaaaatgccgcaaaaaaggga |
| | | ataagggcgacacggaaatgttgaatactcatactcttcctttttcaatattattgaagcatttatcagggtt |
| | | attgtctcatgagcggatacatatttgaatgtatttagaaaaataaacaaataggggtacgcgtaaattgta |
| | | agcgttaatattttgttaaaattcgcgttaaatttttgttaaatcagctcattttttaaccaataggccgaaatc |
| | | ggcaaaatcccttataaatcaaaagaatagaccgagatagggttgagtgttgttccagtttggaacaag |
| | | agtccactattaaagaacgtggactccaacgtcaaagggcgaaaaaccgtctatcagggcgatggcc |
| | | cactacgtgaaccatcaccctaatcaagttttttggggtcgaggtgccgtaaagcactaaatcggaacc |
| | | ctaaagggagcccccgatttagagcttgacggggaaagccggcgaacgtggcgagaaaggaaggg |
| | | aagaaagcgaaaggagcgggcgctagggcgctggcaagtgtagcggtcacgctgcgcgtaaccac |
| | | cacacccgccgcgcttaatgcgccgctacagggcgcgtcaggtggcacttttcggggaaatgtgcgc |
| | | ggaacccctatttgtttatttttctaaatacattcaaatatgtatccgctcatgagacaataaccctgataaat |
| | | gcttcaataatattgaaaaaggaagaatcctgaggcggaaagaaccagctgtggaatgtgtgtcagtta |
| | | gggtgtggaaagtccccaggctccccagcaggcagaagtatgcaaagcatgcatctcaattagtcag |
| | | caaccaggtgtggaaagtccccaggctccccagcaggcagaagtatgcaaagcatgcatctcaatta |
| | | gtcagcaaccatagtcccgcccctaactccgcccatcccgcccctaactccgcccagttccgcccattc |
| | | tccgccccatggctgactaattttttttatttatgcagaggccgaggccgcctcggcctctgagctattcc |
| | | agaagtagtgaggaggcttttttggaggcctaggcttttgcaaatatcgatatgaccgagtacaagccc |
| | | acggtgcgcctcgccacccgcgacgacgtcccccgggccgtacgcaccctcgccgccgcgttcgc |
| | | cgactaccccgccacgcgccacaccgtcgacccggaccgccacatcgagcgggtcaccgagctgc |
| | | aagaactcttcctcacgcgcgtcgggctcgacatcggcaaggtgtgggtcgcggacgacggcgccg |
| | | cggtggcggtctggaccacgccggagagcgtcgaagcgggggcggtgttcgccgagatcggccc |
| | | gcgcatggccgagttgagcggttcccggctggccgcgcagcaacagatggaaggcctcctggcgc |
| | | cgcaccggcccaaggagcccgcgtggttcctggccaccgtcggcgtctcgcccgaccaccagggc |
| | | aagggtctgggcagcgccgtcgtgctccccggagtggaggcggccgagcgcgccggggtgcccg |
| | | ccttcctggagacctccgcgccccgcaacctccccttctacgagcggctcggcttcaccgtcaccgcc |
| | | gacgtcgaggtgcccgaaggaccgcgcacctggtgcatgacccgcaagcccggtgcctgacttaag |
| | | agagcgggactctggggttcgaaatgaccgaccaagcgacgcccaacctgccatcacgagatttcga |
| | | ttccaccgccgccttctatgaaaggttgggcttcggaatcgttttccgggacgccggctggatgatcctc |
| | | cagcgcggggatctcatgctggagttcttcgcccaccctagggggaggctaactgaaacacggaag |
| | | gagacaataccggaaggaacccgcgctatgacggcaataaaaagacagaataaaacgcacggtgtt |
| | | gggtcgtttgttcataaacgcggggttcggtcccagggctggcactctgtcgataccccaccgagacc |
| | | ccattggggccaatacgcccgcgtttcttccttttccccaccccaccccccaagttcgggtgaaggccc |
| | | agggctcgcagccaacgtcggggcggcaggccctgccatagcctcaggttactcatatatactttaga |
| | | ttgatttaaaacttcatttttaatttaaaaggatctaggtgaagatcctttttgataatctcatgaccaaaatcc |
| | | cttaacgtgagttttcgttccactgagcgtcagaccccgtagaaaagatcaaaggatcttcttgagatcct |
| | | ttttttctgcgcgtaatctgctgcttgcaaacaaaaaaaccaccgctaccagcggtggtttgtttgccgga |
| | | tcaagagctaccaactctttttccgaaggtaactggcttcagcagagcgcagataccaaatactgtcctt |
| | | ctagtgtagccgtagttaggccaccacttcaagaactctgtagcaccgcctacatacctcgctctgctaa |
| | | tcctgttaccagtggctgctgccagtggcgataagtcgtgtcttaccgggttggactcaagacgatagtt |
| | | accggataaggcgcagcggtcgggctgaacggggggttcgtgcacacagcccagcttggagcgaa |
| | | cgacctacaccgaactgagatacctacagcgtgagctatgagaaagcgccacgcttcccgaaggga |
| | | gaaaggcggacaggtatccggtaagcggcagggtcggaacaggagagcgcacgagggagcttcc |
| | | agggggaaacgcctggtatctttatagtcctgtcgggtttcgccacctctgacttgagcgtcgatttttgtg |
| | | atgctcgtcaggggggcggagcctatggaaaaacgccagcaacgcggcctttttacggttcctggcct |
| | | tttgctggccttttgctcacatgttctttcctgcgttatcccctgattctgtggataaccgtattaccgcc |
| 6 | plasmid | atgcattagttattaatagtaatcaattacggggtcattagttcatagcccatatatggagttccgcgttaca |
| | pCP5-P1 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggaggcaggtggacaggaaggttc |
| | | taatgttcttaaggcacaggaactgggacatctgggcccggaaagcctttttctctgtgatccggtacag |
| | | tccttctgcggtaccaggtaagtgtacccaattcgccctatagtgagtcgtattacaattcactcgatcgc |
| | | ccttcccaacagttgcgcagcctgaatggcgaatggagatccaatttttaagtgtataatgtgttaaacta |
| | | ctgattctaattgtttgtgtattttagattcacagtcccaaggctcatttcaggcccctcagtcctcacagtct |
| | | gttcatgatcataatcagccataccacatttgtagaggttttacttgctttaaaaaacctcccacacctccc |
| | | cctgaacctgaaacataaaatgaatgcaattgttgttgttaacttgtttattgcagcttataatggttacaaat |
| | | aaagcaatagcatcacaaatttcacaaataaagcatttttttcactgcattctagttgtggtttgtccaaact |
| | | catcaatgtatcttaacgcgtttaccaatgcttaatcagtgaggcacctatctcagcgatctgtctatttcgt |
| | | tcatccatagttgcctgactccccgtcgtgtagataactacgatacgggagggcttaccatctggcccca |
| | | gtgctgcaatgataccgcgagacccacgctcaccggctccagatttatcagcaataaaccagccagcc |
| | | ggaagggccgagcgcagaagtggtcctgcaactttatccgcctccatccagtctattaattgttgccgg |
| | | gaagctagagtaagtagttcgccagttaatagtttgcgcaacgttgttgccattgctacaggcatcgtggt |
| | | gtcacgctcgtcgtttggtatggcttcattcagctccggttcccaacgatcaaggcgagttacatgatccc |
| | | ccatgttgtgcaaaaaagcggttagctccttcggtcctccgatcgttgtcagaagtaagttggccgcagt |
| | | gttatcactcatggttatggcagcactgcataattctcttactgtcatgccatccgtaagatgcttttctgtga |
| | | ctggtgagtactcaaccaagtcattctgagaatagtgtatgcggcgaccgagttgctcttgcccggcgt |
| | | caatacgggataataccgcgccacatagcagaactttaaaagtgctcatcattggaaaacgttcttcgg |
| | | ggcgaaaactctcaaggatcttaccgctgttgagatccagttcgatgtaacccactcgtgcacccaact |
| | | gatcttcagcatcttttactttcaccagcgtttctgggtgagcaaaaacaggaaggcaaaatgccgcaaa |
| | | aaagggaataagggcgacacggaaatgttgaatactcatactcttcctttttcaatattattgaagcatttat |
| | | cagggttattgtctcatgagcggatacatatttgaatgtatttagaaaaataaacaaataggggtacgcgt |
| | | aaattgtaagcgttaatattttgttaaaattcgcgttaaatttttgttaaatcagctcattttttaaccaataggc |
| | | cgaaatcggcaaaatcccttataaatcaaaagaatagaccgagatagggttgagtgttgttccagtttgg |
| | | aacaagagtccactattaaagaacgtggactccaacgtcaaagggcgaaaaaccgtctatcagggcg |
| | | atggcccactacgtgaaccatcaccctaatcaagttttttggggtcgaggtgccgtaaagcactaaatcg |
| | | gaaccctaaagggagcccccgatttagagcttgacggggaaagccggcgaacgtggcgagaaagg |
| | | aagggaagaaagcgaaaggagcgggcgctagggcgctggcaagtgtagcggtcacgctgcgcgt |
| | | aaccaccacacccgccgcgcttaatgcgccgctacagggcgcgtcaggtggcacttttcggggaaat |
| | | gtgcgcggaacccctatttgtttatttttctaaatacattcaaatatgtatccgctcatgagacaataaccct |
| | | gataaatgcttcaataatattgaaaaaggaagaatcctgaggcggaaagaaccagctgtggaatgtgt |
| | | gtcagttagggtgtggaaagtccccaggctccccagcaggcagaagtatgcaaagcatgcatctcaat |
| | | tagtcagcaaccaggtgtggaaagtccccaggctccccagcaggcagaagtatgcaaagcatgcatc |
| | | tcaattagtcagcaaccatagtcccgcccctaactccgcccatcccgcccctaactccgcccagttccg |
| | | cccattctccgccccatggctgactaattttttttatttatgcagaggccgaggccgcctcggcctctgag |
| | | ctattccagaagtagtgaggaggcttttttggaggcctaggcttttgcaaatatcgatatgaccgagtaca |
| | | agcccacggtgcgcctcgccacccgcgacgacgtcccccgggccgtacgcaccctcgccgccgcg |
| | | ttcgccgactaccccgccacgcgccacaccgtcgacccggaccgccacatcgagcgggtcaccga |
| | | gctgcaagaactcttcctcacgcgcgtcgggctcgacatcggcaaggtgtgggtcgcggacgacgg |
| | | cgccgcggtggcggtctggaccacgccggagagcgtcgaagcgggggcggtgttcgccgagatc |
| | | ggcccgcgcatggccgagttgagcggttcccggctggccgcgcagcaacagatggaaggcctcct |
| | | ggcgccgcaccggcccaaggagcccgcgtggttcctggccaccgtcggcgtctcgcccgaccacc |
| | | agggcaagggtctgggcagcgccgtcgtgctccccggagtggaggcggccgagcgcgccggggt |
| | | gcccgccttcctggagacctccgcgccccgcaacctccccttctacgagcggctcggcttcaccgtca |
| | | ccgccgacgtcgaggtgcccgaaggaccgcgcacctggtgcatgacccgcaagcccggtgcctga |
| | | cttaagagcgggactctggggttcgaaatgaccgaccaagcgacgcccaacctgccatcacgagatt |
| | | tcgattccaccgccgccttctatgaaaggttgggcttcggaatcgttttccgggacgccggctggatgat |
| | | cctccagcgcggggatctcatgctggagttcttcgcccaccctagggggaggctaactgaaacacgg |
| | | aaggagacaataccggaaggaacccgcgctatgacggcaataaaaagacagaataaaacgcacgg |
| | | tgttgggtcgtttgttcataaacgcggggttcggtcccagggctggcactctgtcgataccccaccgag |
| | | accccattggggccaatacgcccgcgtttcttccttttccccaccccaccccccaagttcgggtgaagg |
| | | cccagggctcgcagccaacgtcggggcggcaggccctgccatagcctcaggttactcatatatacttt |
| | | agattgatttaaaacttcatttttaatttaaaaggatctaggtgaagatcctttttgataatctcatgaccaaa |
| | | atcccttaacgtgagttttcgttccactgagcgtcagaccccgtagaaaagatcaaaggatcttcttgag |
| | | atcctttttttctgcgcgtaatctgctgcttgcaaacaaaaaaaccaccgctaccagcggtggtttgtttgc |
| | | cggatcaagagctaccaactctttttccgaaggtaactggcttcagcagagcgcagataccaaatactg |
| | | tccttctagtgtagccgtagttaggccaccacttcaagaactctgtagcaccgcctacatacctcgctctg |
| | | ctaatcctgttaccagtggctgctgccagtggcgataagtcgtgtcttaccgggttggactcaagacgat |
| | | agttaccggataaggcgcagcggtcgggctgaacggggggttcgtgcacacagcccagcttggagc |
| | | gaacgacctacaccgaactgagatacctacagcgtgagctatgagaaagcgccacgcttcccgaag |
| | | ggagaaaggcggacaggtatccggtaagcggcagggtcggaacaggagagcgcacgagggagc |
| | | ttccagggggaaacgcctggtatctttatagtcctgtcgggtttcgccacctctgacttgagcgtcgatttt |
| | | tgtgatgctcgtcaggggggggagcctatggaaaaacgccagcaacgcggcctttttacggttcctg |
| | | gccttttgctggccttttgctcacatgttctttcctgcgttatcccctgattctgtggataaccgtattaccgc |
| | | c |
| 7 | plasmid | atgcattagttattaatagtaatcaattacggggtcattagttcatagcccatatatggagttccgcgttaca |
| | pCP5-P2 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggtaccaagcttcgaggcaggtgg |
| | | acagcttggttctaatgaagttaaccctgtcgttctgcgacatctgggcccggaaagcgtttaactgatg |
| | | gatggaacagtccttctgcggtaccaggtaagtgtacccaattcgccctatagtgagtcgtattacaattc |
| | | actcgatcgcccttcccaacagttgcgcagcctgaatggcgaatggagatccaatttttaagtgtataat |
| | | gtgttaaactactgattctaattgtttgtgtattttagattcacagtcccaaggctcatttcaggcccctcagt |
| | | cctcacagtctgttcatgatcataatcagccataccacatttgtagaggttttacttgctttaaaaaacctcc |
| | | cacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtt |
| | | tgtccaaactcatcaatgtatcttaacgcgtttaccaatgcttaatcagtgaggcacctatctcagcgatct |
| | | gtctatttcgttcatccatagttgcctgactccccgtcgtgtagataactacgatacgggagggcttacca |
| | | tctggccccagtgctgcaatgataccgcgagacccacgctcaccggctccagatttatcagcaataaa |
| | | ccagccagccggaagggccgagcgcagaagtggtcctgcaactttatccgcctccatccagtctatta |
| | | attgttgccgggaagctagagtaagtagttcgccagttaatagtttgcgcaacgttgttgccattgctaca |
| | | ggcatcgtggtgtcacgctcgtcgtttggtatggcttcattcagctccggttcccaacgatcaaggcgag |
| | | ttacatgatcccccatgttgtgcaaaaaagcggttagctccttcggtcctccgatcgttgtcagaagtaag |
| | | ttggccgcagtgttatcactcatggttatggcagcactgcataattctcttactgtcatgccatccgtaaga |
| | | tgcttttctgtgactggtgagtactcaaccaagtcattctgagaatagtgtatgcggcgaccgagttgctc |
| | | ttgcccggcgtcaatacgggataataccgcgccacatagcagaactttaaaagtgctcatcattggaaa |
| | | acgttcttcggggcgaaaactctcaaggatcttaccgctgttgagatccagttcgatgtaacccactcgt |
| | | gcacccaactgatcttcagcatcttttactttcaccagcgtttctgggtgagcaaaaacaggaaggcaaa |
| | | atgccgcaaaaaagggaataagggcgacacggaaatgttgaatactcatactcttcctttttcaatattat |
| | | tgaagcatttatcagggttattgtctcatgagcggatacatatttgaatgtatttagaaaaataaacaaata |
| | | ggggtacgcgtaaattgtaagcgttaatattttgttaaaattcgcgttaaatttttgttaaatcagctcatttttt |
| | | aaccaataggccgaaatcggcaaaatcccttataaatcaaaagaatagaccgagatagggttgagtgtt |
| | | gttccagtttggaacaagagtccactattaaagaacgtggactccaacgtcaaagggcgaaaaaccgt |
| | | ctatcagggcgatggcccactacgtgaaccatcaccctaatcaagttttttggggtcgaggtgccgtaa |
| | | agcactaaatcggaaccctaaagggagcccccgatttagagcttgacggggaaagccggcgaacgt |
| | | ggcgagaaaggaagggaagaaagcgaaaggagcgggcgctagggcgctggcaagtgtagcggt |
| | | cacgctgcgcgtaaccaccacacccgccgcgcttaatgcgccgctacagggcgcgtcaggtggcac |
| | | ttttcggggaaatgtgcgcggaacccctatttgtttatttttctaaatacattcaaatatgtatccgctcatga |
| | | gacaataaccctgataaatgcttcaataatattgaaaaaggaagaatcctgaggcggaaagaaccagc |
| | | tgtggaatgtgtgtcagttagggtgtggaaagtccccaggctccccagcaggcagaagtatgcaaagc |
| | | atgcatctcaattagtcagcaaccaggtgtggaaagtccccaggctccccagcaggcagaagtatgca |
| | | aagcatgcatctcaattagtcagcaaccatagtcccgcccctaactccgcccatcccgcccctaactcc |
| | | gcccagttccgcccattctccgccccatggctgactaattttttttatttatgcagaggccgaggccgcct |
| | | cggcctctgagctattccagaagtagtgaggaggcttttttggaggcctaggcttttgcaaatatcgatat |
| | | gaccgagtacaagcccacggtgcgcctcgccacccgcgacgacgtcccccgggccgtacgcaccc |
| | | tcgccgccgcgttcgccgactaccccgccacgcgccacaccgtcgacccggaccgccacatcgag |
| | | cgggtcaccgagctgcaagaactcttcctcacgcgcgtcgggctcgacatcggcaaggtgtgggtcg |
| | | cggacgacggcgccgcggtggcggtctggaccacgccggagagcgtcgaagcgggggcggtgtt |
| | | cgccgagatcggcccgcgcatggccgagttgagcggttcccggctggccgcgcagcaacagatgg |
| | | aaggcctcctggcgccgcaccggcccaaggagcccgcgtggttcctggccaccgtcggcgtctcgc |
| | | ccgaccaccagggcaagggtctgggcagcgccgtcgtgctccccggagtggaggcggccgagcg |
| | | cgccggggtgcccgccttcctggagacctccgcgccccgcaacctccccttctacgagcggctcggc |
| | | ttcaccgtcaccgccgacgtcgaggtgcccgaaggaccgcgcacctggtgcatgacccgcaagccc |
| | | ggtgcctgacttaagagcgggactctggggttcgaaatgaccgaccaagcgacgcccaacctgccat |
| | | cacgagatttcgattccaccgccgccttctatgaaaggttgggcttcggaatcgttttccgggacgccg |
| | | gctggatgatcctccagcgcggggatctcatgctggagttcttcgcccaccctagggggaggctaact |
| | | gaaacacggaaggagacaataccggaaggaacccgcgctatgacggcaataaaaagacagaataa |
| | | aacgcacggtgttgggtcgtttgttcataaacgcggggttcggtcccagggctggcactctgtcgatac |
| | | cccaccgagaccccattggggccaatacgcccgcgtttcttccttttccccaccccaccccccaagttc |
| | | gggtgaaggcccagggctcgcagccaacgtcggggggcaggccctgccatagcctcaggttactc |
| | | atatatactttagattgatttaaaacttcatttttaatttaaaaggatctaggtgaagatcctttttgataatctc |
| | | atgaccaaaatcccttaacgtgagttttcgttccactgagcgtcagaccccgtagaaaagatcaaagga |
| | | tcttcttgagatcctttttttctgcgcgtaatctgctgcttgcaaacaaaaaaaccaccgctaccagcggtg |
| | | gtttgtttgccggatcaagagctaccaactctttttccgaaggtaactggcttcagcagagcgcagatac |
| | | caaatactgtccttctagtgtagccgtagttaggccaccacttcaagaactctgtagcaccgcctacata |
| | | cctcgctctgctaatcctgttaccagtggctgctgccagtggcgataagtcgtgtcttaccgggttggact |
| | | caagacgatagttaccggataaggcgcagcggtcgggctgaacggggggttcgtgcacacagccca |
| | | gcttggagcgaacgacctacaccgaactgagatacctacagcgtgagctatgagaaagcgccacgct |
| | | tcccgaagggagaaaggcggacaggtatccggtaagcggcagggtcggaacaggagagcgcacg |
| | | agggagcttccagggggaaacgcctggtatctttatagtcctgtcgggtttcgccacctctgacttgagc |
| | | gtcgatttttgtgatgctcgtcaggggggcggagcctatggaaaaacgccagcaacgcggcctttttac |
| | | ggttcctggccttttgctggccttttgctcacatgttctttcctgcgttatcccctgattctgtggataaccgt |
| | | attaccgcc |
| 8 | plasmid | atgcattagttattaatagtaatcaattacggggtcattagttcatagcccatatatggagttccgcgttaca |
| | pCP5-P3 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaaggg |
| | | cgaattcggatccgcggccgccttaagctcgaggcaggtggacaggaaggttctaatgttctataggg |
| | | tctgcttgtcgctcatctgggcccggagatgcgtaaagtcagacatccggtacagtccttctgcggtacc |
| | | aggtaagtgtacccaattcgccctatagtgagtcgtattacaattcactcgatcgcccttcccaacagttg |
| | | cgcagcctgaatggcgaatggagatccaatttttaagtgtataatgtgttaaactactgattctaattgtttg |
| | | tgtattttagattcacagtcccaaggctcatttcaggcccctcagtcctcacagtctgttcatgatcataatc |
| | | agccataccacatttgtagaggttttacttgctttaaaaaacctcccacacctccccctgaacctgaaaca |
| | | taaaatgaatgcaattgttgttgttaacttgtttattgcagcttataatggttacaaataaagcaatagcatca |
| | | caaatttcacaaataaagcatttttttcactgcattctagttgtggtttgtccaaactcatcaatgtatcttaac |
| | | gcgtttaccaatgcttaatcagtgaggcacctatctcagcgatctgtctatttcgttcatccatagttgcctg |
| | | actccccgtcgtgtagataactacgatacgggagggcttaccatctggccccagtgctgcaatgatacc |
| | | gcgagacccacgctcaccggctccagatttatcagcaataaaccagccagccggaagggccgagcg |
| | | cagaagtggtcctgcaactttatccgcctccatccagtctattaattgttgccgggaagctagagtaagta |
| | | gttcgccagttaatagtttgcgcaacgttgttgccattgctacaggcatcgtggtgtcacgctcgtcgtttg |
| | | gtatggcttcattcagctccggttcccaacgatcaaggcgagttacatgatcccccatgttgtgcaaaaa |
| | | agcggttagctccttcggtcctccgatcgttgtcagaagtaagttggccgcagtgttatcactcatggtta |
| | | tggcagcactgcataattctcttactgtcatgccatccgtaagatgcttttctgtgactggtgagtactcaa |
| | | ccaagtcattctgagaatagtgtatgcggcgaccgagttgctcttgcccggcgtcaatacgggataata |
| | | ccgcgccacatagcagaactttaaaagtgctcatcattggaaaacgttcttcggggcgaaaactctcaa |
| | | ggatcttaccgctgttgagatccagttcgatgtaacccactcgtgcacccaactgatcttcagcatctttta |
| | | ctttcaccagcgtttctgggtgagcaaaaacaggaaggcaaaatgccgcaaaaaagggaataagggc |
| | | gacacggaaatgttgaatactcatactcttcctttttcaatattattgaagcatttatcagggttattgtctcat |
| | | gagcggatacatatttgaatgtatttagaaaaataaacaaataggggtacgcgtaaattgtaagcgttaat |
| | | attttgttaaaattcgcgttaaatttttgttaaatcagctcattttttaaccaataggccgaaatcggcaaaat |
| | | cccttataaatcaaaagaatagaccgagatagggttgagtgttgttccagtttggaacaagagtccacta |
| | | ttaaagaacgtggactccaacgtcaaagggcgaaaaaccgtctatcagggcgatggcccactacgtg |
| | | aaccatcaccctaatcaagttttttggggtcgaggtgccgtaaagcactaaatcggaaccctaaaggga |
| | | gcccccgatttagagcttgacggggaaagccggcgaacgtggcgagaaaggaagggaagaaagc |
| | | gaaaggagcgggcgctagggcgctggcaagtgtagcggtcacgctgcgcgtaaccaccacacccg |
| | | ccgcgcttaatgcgccgctacagggcgcgtcaggtggcacttttcggggaaatgtgcgcggaacccc |
| | | tatttgtttatttttctaaatacattcaaatatgtatccgctcatgagacaataaccctgataaatgcttcaata |
| | | atattgaaaaaggaagaatcctgaggcggaaagaaccagctgtggaatgtgtgtcagttagggtgtgg |
| | | aaagtccccaggctccccagcaggcagaagtatgcaaagcatgcatctcaattagtcagcaaccagg |
| | | tgtggaaagtccccaggctccccagcaggcagaagtatgcaaagcatgcatctcaattagtcagcaac |
| | | catagtcccgcccctaactccgcccatcccgcccctaactccgcccagttccgcccattctccgcccca |
| | | tggctgactaattttttttatttatgcagaggccgaggccgcctcggcctctgagctattccagaagtagt |
| | | gaggaggcttttttggaggcctaggcttttgcaaatatcgatatgaccgagtacaagcccacggtgcgc |
| | | ctcgccacccgcgacgacgtcccccgggccgtacgcaccctcgccgccgcgttcgccgactacccc |
| | | gccacgcgccacaccgtcgacccggaccgccacatcgagcgggtcaccgagctgcaagaactcttc |
| | | ctcacgcgcgtcgggctcgacatcggcaaggtgtgggtcgcggacgacggcgccgcggtggcggt |
| | | ctggaccacgccggagagcgtcgaagcgggggcggtgttcgccgagatcggcccgcgcatggcc |
| | | gagttgagcggttcccggctggccgcgcagcaacagatggaaggcctcctggcgccgcaccggcc |
| | | caaggagcccgcgtggttcctggccaccgtcggcgtctcgcccgaccaccagggcaagggtctggg |
| | | cagcgccgtcgtgctccccggagtggaggcggccgagcgcgccggggtgcccgccttcctggaga |
| | | cctccgcgccccgcaacctccccttctacgagcggctcggcttcaccgtcaccgccgacgtcgaggt |
| | | gcccgaaggaccgcgcacctggtgcatgacccgcaagcccggtgcctgacttaagagcgggactct |
| | | ggggttcgaaatgaccgaccaagcgacgcccaacctgccatcacgagatttcgattccaccgccgcc |
| | | ttctatgaaaggttgggcttcggaatcgttttccgggacgccggctggatgatcctccagcgcggggat |
| | | ctcatgctggagttcttcgcccaccctagggggaggctaactgaaacacggaaggagacaataccgg |
| | | aaggaacccgcgctatgacggcaataaaaagacagaataaaacgcacggtgttgggtcgtttgttcat |
| | | aaacgcggggttcggtcccagggctggcactctgtcgataccccaccgagaccccattggggccaat |
| | | acgcccgcgtttcttccttttccccaccccaccccccaagttcgggtgaaggcccagggctgcagcc |
| | | aacgtcggggcggcaggccctgccatagcctcaggttactcatatatactttagattgatttaaaacttca |
| | | tttttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgtaat |
| | | ctgctgcttgcaaacaaaaaaaccaccgctaccagcggtggtttgtttgccggatcaagagctaccaac |
| | | tctttttccgaaggtaactggcttcagcagagcgcagataccaaatactgtccttctagtgtagccgtagt |
| | | taggccaccacttcaagaactctgtagcaccgcctacatacctcgctctgctaatcctgttaccagtggc |
| | | tgctgccagtggcgataagtcgtgtcttaccgggttggactcaagacgatagttaccggataaggcgc |
| | | agcggtcgggctgaacggggggttcgtgcacacagcccagcttggagcgaacgacctacaccgaa |
| | | ctgagatacctacagcgtgagctatgagaaagcgccacgcttcccgaagggagaaaggcggacag |
| | | gtatccggtaagcggcagggtcggaacaggagagcgcacgagggagcttccagggggaaacgcc |
| | | tggtatctttatagtcctgtcgggtttcgccacctctgacttgagcgtcgatttttgtgatgctcgtcagggg |
| | | ggcggagcctatggaaaaacgccagcaacgcggcctttttacggttcctggccttttgctggccttttgc |
| | | tcacatgttctttcctgcgttatcccctgattctgtggataaccgtattaccgcc |
| 9 | plasmid | atgcattagttattaatagtaatcaattacggggtcattagttcatagcccatatatggagttccgcgttaca |
| | pCP4-H | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaccgg |
| | | tggtaccaggtaagtgtacccaattcgccctatagtgagtcgtattacaattcactcgatcgcccttccca |
| | | acagttgcgcagcctgaatggcgaatggagatccaatttttaagtgtataatgtgttaaactactgattcta |
| | | attgtttgtgtattttagattcacagtcccaaggctcatttcaggcccctcagtcctcacagtctgttcatgat |
| | | cataatcagccataccacatttgtagaggttttacttgctttaaaaaacctcccacacctccccctgaacct |
| | | gaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgt |
| | | atcttaacgcgtttaccaatgcttaatcagtgaggcacctatctcagcgatctgtctatttcgttcatccata |
| | | gttgcctgactccccgtcgtgtagataactacgatacgggagggcttaccatctggccccagtgctgca |
| | | atgataccgcgagacccacgctcaccggctccagatttatcagcaataaaccagccagccggaaggg |
| | | ccgagcgcagaagtggtcctgcaactttatccgcctccatccagtctattaattgttgccgggaagctag |
| | | agtaagtagttcgccagttaatagtttgcgcaacgttgttgccattgctacaggcatcgtggtgtcacgct |
| | | cgtcgtttggtatggcttcattcagctccggttcccaacgatcaaggcgagttacatgatcccccatgttg |
| | | tgcaaaaaagcggttagctccttcggtcctccgatcgttgtcagaagtaagttggccgcagtgttatcac |
| | | tcatggttatggcagcactgcataattctcttactgtcatgccatccgtaagatgcttttctgtgactggtga |
| | | gtactcaaccaagtcattctgagaatagtgtatgcggcgaccgagttgctcttgcccggcgtcaatacg |
| | | ggataataccgcgccacatagcagaactttaaaagtgctcatcattggaaaacgttcttcggggcgaaa |
| | | actctcaaggatcttaccgctgttgagatccagttcgatgtaacccactcgtgcacccaactgatcttcag |
| | | catcttttactttcaccagcgtttctgggtgagcaaaaacaggaaggcaaaatgccgcaaaaaaggga |
| | | ataagggcgacacggaaatgttgaatactcatactcttcctttttcaatattattgaagcatttatcagggtt |
| | | attgtctcatgagcggatacatatttgaatgtatttagaaaaataaacaaataggggtacgcgtaaattgta |
| | | agcgttaatattttgttaaaattcgcgttaaatttttgttaaatcagctcattttttaaccaataggccgaaatc |
| | | ggcaaaatcccttataaatcaaaagaatagaccgagatagggttgagtgttgttccagtttggaacaag |
| | | agtccactattaaagaacgtggactccaacgtcaaagggcgaaaaaccgtctatcagggcgatggcc |
| | | cactacgtgaaccatcaccctaatcaagttttttggggtcgaggtgccgtaaagcactaaatcggaacc |
| | | ctaaagggagcccccgatttagagcttgacggggaaagccggcgaacgtggcgagaaaggaaggg |
| | | aagaaagcgaaaggagcgggcgctagggcgctggcaagtgtagcggtcacgctgcgcgtaaccac |
| | | cacacccgccgcgcttaatgcgccgctacagggcgcgtcaggtggcacttttcggggaaatgtgcgc |
| | | ggaacccctatttgtttatttttctaaatacattcaaatatgtatccgctcatgagacaataaccctgataaat |
| | | gcttcaataatattgaaaaaggaagaatcctgaggcggaaagaaccagctgtggaatgtgtgtcagtta |
| | | gggtgtggaaagtccccaggctccccagcaggcagaagtatgcaaagcatgcatctcaattagtcag |
| | | caaccaggtgtggaaagtccccaggctccccagcaggcagaagtatgcaaagcatgcatctcaatta |
| | | gtcagcaaccatagtcccgcccctaactccgcccatcccgcccctaactccgcccagttccgcccattc |
| | | tccgccccatggctgactaattttttttatttatgcagaggccgaggccgcctcggcctctgagctattcc |
| | | agaagtagtgaggaggcttttttggaggcctaggcttttgcaaatatcgatatgaaaaagcctgaactca |
| | | ccgcgacgtctgtcgagaagtttctgatcgaaaagttcgacagcgtctccgacctgatgcagctctcgg |
| | | agggcgaagaatctcgtgctttcagcttcgatgtaggagggcgtggatatgtcctgcgggtaaatagct |
| | | gcgccgatggtttctacaaagatcgttatgtttatcggcactttgcatcggccgcgctcccgattccgga |
| | | agtgcttgacattggggaattcagcgagagcctgacctattgcatctcccgccgtgcacagggtgtcac |
| | | gttgcaagacctgcctgaaaccgaactgcccgctgttctgcagccggtcgcggaggccatggatgcg |
| | | atcgctgcggccgatcttagccagacgagcgggttcggcccattcggaccgcaaggaatcggtcaat |
| | | acactacatggcgtgatttcatatgcgcgattgctgatccccatgtgtatcactggcaaactgtgatggac |
| | | gacaccgtcagtgcgtccgtcgcgcaggctctcgatgagctgatgctttgggccgaggactgccccg |
| | | aagtccggcacctcgtgcacgcggatttcggctccaacaatgtcctgacggacaatggccgcataaca |
| | | gcggtcattgactggagcgaggcgatgttcggggattcccaatacgaggtcgccaacatcttcttctgg |
| | | aggccgtggttggcttgtatggagcagcagacgcgctacttcgagcggaggcatccggagcttgcag |
| | | gatcgccgcggctccgggcgtatatgctccgcattggtcttgaccaactctatcagagcttggttgacg |
| | | gcaatttcgatgatgcagcttgggcgcagggtcgatgcgacgcaatcgtccgatccggagccgggac |
| | | tgtcgggcgtacacaaatcgcccgcagaagcgcggccgtctggaccgatggctgtgtagaagtactc |
| | | gccgatagtggaaaccgacgccccagcactcgtccgagggcaaaggaatagtgagcgggactctg |
| | | gggttcgaaatgaccgaccaagcgacgcccaacctgccatcacgagatttcgattccaccgccgcctt |
| | | ctatgaaaggttgggcttcggaatcgttttccgggacgccggctggatgatcctccagcgcggggatct |
| | | catgctggagttcttcgcccaccctagggggaggctaactgaaacacggaaggagacaataccgga |
| | | aggaacccgcgctatgacggcaataaaaagacagaataaaacgcacggtgttgggtcgtttgttcata |
| | | aacgcggggttcggtcccagggctggcactctgtcgataccccaccgagaccccattggggccaata |
| | | cgcccgcgtttcttccttttccccaccccaccccccaagttcgggtgaaggcccagggctcgcagcca |
| | | acgtcggggcggcaggccctgccatagcctcaggttactcatatatactttagattgatttaaaacttcatt |
| | | tttaatttaaaaggatctaggtgaagatcctttttgataatctcatgaccaaaatcccttaacgtgagttttcg |
| | | ttccactgagcgtcagaccccgtagaaaagatcaaaggatcttcttgagatcctttttttctgcgcgtaatc |
| | | tgctgcttgcaaacaaaaaaaccaccgctaccagcggtggtttgtttgccggatcaagagctaccaact |
| | | ctttttccgaaggtaactggcttcagcagagcgcagataccaaatactgtccttctagtgtagccgtagtt |
| | | aggccaccacttcaagaactctgtagcaccgcctacatacctcgctctgctaatcctgttaccagtggct |
| | | gctgccagtggcgataagtcgtgtcttaccgggttggactcaagacgatagttaccggataaggcgca |
| | | gcggtcgggctgaacggggggttcgtgcacacagcccagcttggagcgaacgacctacaccgaact |
| | | gagatacctacagcgtgagctatgagaaagcgccacgcttcccgaagggagaaaggcggacaggt |
| | | atccggtaagcggcagggtcggaacaggagagcgcacgagggagcttccagggggaaacgcctg |
| | | gtatctttatagtcctgtcgggtttcgccacctctgacttgagcgtcgatttttgtgatgctcgtcaggggg |
| | | gcggagcctatggaaaaacgccagcaacgcggcctttttacggttcctggccttttgctggccttttgct |
| | | cacatgttctttcctgcgttatcccctgattctgtggataaccgtattaccgcc |
| 10 | plasmid | atgcattagttattaatagtaatcaattacggggtcattagttcatagcccatatatggagttccgcgttaca |
| | pCP4-H1 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggaggcaggtggacaggaaggttc |
| | | taatgttcttaaggcacaggaactgggacatctgggcccggaaagcctttttctctgtgatccggtacag |
| | | tccttctgcggtaccaggtaagtgtacccaattcgccctatagtgagtcgtattacaattcactcgatcgc |
| | | ccttcccaacagttgcgcagcctgaatggcgaatggagatccaatttttaagtgtataatgtgttaaacta |
| | | ctgattctaattgtttgtgtattttagattcacagtcccaaggctcatttcaggcccctcagtcctcacagtct |
| | | gttcatgatcataatcagccataccacatttgtagaggttttacttgctttaaaaaacctcccacacctccc |
| | | cctgaacctgaaacataaaatgaatgcaattgttgttgttaacttgtttattgcagcttataatggttacaaat |
| | | aaagcaatagcatcacaaatttcacaaataaagcatttttttcactgcattctagttgtggtttgtccaaact |
| | | catcaatgtatcttaacgcgtttaccaatgcttaatcagtgaggcacctatctcagcgatctgtctatttcgt |
| | | tcatccatagttgcctgactccccgtcgtgtagataactacgatacgggagggcttaccatctggcccca |
| | | gtgctgcaatgataccgcgagacccacgctcaccggctccagatttatcagcaataaaccagccagcc |
| | | ggaagggccgagcgcagaagtggtcctgcaactttatccgcctccatccagtctattaattgttgccgg |
| | | gaagctagagtaagtagttcgccagttaatagtttgcgcaacgttgttgccattgctacaggcatcgtggt |
| | | gtcacgctcgtcgtttggtatggcttcattcagctccggttcccaacgatcaaggcgagttacatgatccc |
| | | ccatgttgtgcaaaaaagcggttagctccttcggtcctccgatcgttgtcagaagtaagttggccgcagt |
| | | gttatcactcatggttatggcagcactgcataattctcttactgtcatgccatccgtaagatgcttttctgtga |
| | | ctggtgagtactcaaccaagtcattctgagaatagtgtatgcggcgaccgagttgctcttgcccggcgt |
| | | caatacgggataataccgcgccacatagcagaactttaaaagtgctcatcattggaaaacgttcttcgg |
| | | ggcgaaaactctcaaggatcttaccgctgttgagatccagttcgatgtaacccactcgtgcacccaact |
| | | gatcttcagcatcttttactttcaccagcgtttctgggtgagcaaaaacaggaaggcaaaatgccgcaaa |
| | | aaagggaataagggcgacacggaaatgttgaatactcatactcttcctttttcaatattattgaagcatttat |
| | | cagggttattgtctcatgagcggatacatatttgaatgtatttagaaaaataaacaaataggggtacgcgt |
| | | aaattgtaagcgttaatattttgttaaaattcgcgttaaatttttgttaaatcagctcattttttaaccaataggc |
| | | cgaaatcggcaaaatcccttataaatcaaaagaatagaccgagatagggttgagtgttgttccagtttgg |
| | | aacaagagtccactattaaagaacgtggactccaacgtcaaagggcgaaaaaccgtctatcagggcg |
| | | atggcccactacgtgaaccatcaccctaatcaagttttttggggtcgaggtgccgtaaagcactaaatcg |
| | | gaaccctaaagggagcccccgatttagagcttgacggggaaagccggcgaacgtggcgagaaagg |
| | | aagggaagaaagcgaaaggagcgggcgctagggcgctggcaagtgtagcggtcacgctgcgcgt |
| | | aaccaccacacccgccgcgcttaatgcgccgctacagggcgcgtcaggtggcacttttcggggaaat |
| | | gtgcgcggaacccctatttgtttatttttctaaatacattcaaatatgtatccgctcatgagacaataaccct |
| | | gataaatgcttcaataatattgaaaaaggaagaatcctgaggcggaaagaaccagctgtggaatgtgt |
| | | gtcagttagggtgtggaaagtccccaggctccccagcaggcagaagtatgcaaagcatgcatctcaat |
| | | tagtcagcaaccaggtgtggaaagtccccaggctccccagcaggcagaagtatgcaaagcatgcatc |
| | | tcaattagtcagcaaccatagtcccgcccctaactccgcccatcccgcccctaactccgcccagttccg |
| | | cccattctccgccccatggctgactaattttttttatttatgcagaggccgaggccgcctcggcctctgag |
| | | ctattccagaagtagtgaggaggcttttttggaggcctaggcttttgcaaatatcgatatgaaaaagcctg |
| | | aactcaccgcgacgtctgtcgagaagtttctgatcgaaaagttcgacagcgtctccgacctgatgcagc |
| | | tctcggagggcgaagaatctcgtgctttcagcttcgatgtaggagggcgtggatatgtcctgcgggtaa |
| | | atagctgcgccgatggtttctacaaagatcgttatgtttatcggcactttgcatcggccgcgctcccgatt |
| | | ccggaagtgcttgacattggggaattcagcgagagcctgacctattgcatctcccgccgtgcacaggg |
| | | tgtcacgttgcaagacctgcctgaaaccgaactgcccgctgttctgcagccggtcgcggaggccatg |
| | | gatgcgatcgctgcggccgatcttagccagacgagcgggttcggcccattcggaccgcaaggaatc |
| | | ggtcaatacactacatggcgtgatttcatatgcgcgattgctgatccccatgtgtatcactggcaaactgt |
| | | gatggacgacaccgtcagtgcgtccgtcgcgcaggctctcgatgagctgatgctttgggccgaggac |
| | | tgccccgaagtccggcacctcgtgcacgcggatttcggctccaacaatgtcctgacggacaatggcc |
| | | gcataacagcggtcattgactggagcgaggcgatgttcggggattcccaatacgaggtcgccaacat |
| | | cttcttctggaggccgtggttggcttgtatggagcagcagacgcgctacttcgagcggaggcatccgg |
| | | agcttgcaggatcgccgcggctccgggcgtatatgctccgcattggtcttgaccaactctatcagagctt |
| | | ggttgacggcaatttcgatgatgcagcttgggcgcagggtcgatgcgacgcaatcgtccgatccgga |
| | | gccgggactgtcgggcgtacacaaatcgcccgcagaagcgcggccgtctggaccgatggctgtgta |
| | | gaagtactcgccgatagtggaaaccgacgccccagcactcgtccgagggcaaaggaatagtgagcg |
| | | ggactctggggttcgaaatgaccgaccaagcgacgcccaacctgccatcacgagatttcgattccacc |
| | | gccgccttctatgaaaggttgggcttcggaatcgttttccgggacgccggctggatgatcctccagcgc |
| | | ggggatctcatgctggagttcttcgcccaccctagggggaggctaactgaaacacggaaggagacaa |
| | | taccggaaggaacccgcgctatgacggcaataaaaagacagaataaaacgcacggtgttgggtcgtt |
| | | tgttcataaacgcggggttcggtcccagggctggcactctgtcgataccccaccgagaccccattggg |
| | | gccaatacgcccgcgtttcttccttttccccaccccaccccccaagttcgggtgaaggcccagggctcg |
| | | cagccaacgtcggggcggcaggccctgccatagcctcaggttactcatatatactttagattgatttaaa |
| | | acttcatttttaatttaaaaggatctaggtgaagatcctttttgataatctcatgaccaaaatcccttaacgtg |
| | | agttttcgttccactgagcgtcagaccccgtagaaaagatcaaaggatcttcttgagatcctttttttctgc |
| | | gcgtaatctgctgcttgcaaacaaaaaaaccaccgctaccagcggtggtttgtttgccggatcaagagc |
| | | taccaactctttttccgaaggtaactggcttcagcagagcgcagataccaaatactgtccttctagtgtag |
| | | ccgtagttaggccaccacttcaagaactctgtagcaccgcctacatacctcgctctgctaatcctgttacc |
| | | agtggctgctgccagtggcgataagtcgtgtcttaccgggttggactcaagacgatagttaccggataa |
| | | ggcgcagcggtcgggctgaacggggggttcgtgcacacagcccagcttggagcgaacgacctaca |
| | | ccgaactgagatacctacagcgtgagctatgagaaagcgccacgcttcccgaagggagaaaggcgg |
| | | acaggtatccggtaagcggcagggtcggaacaggagagcgcacgagggagcttccagggggaaa |
| | | cgcctggtatctttatagtcctgtcgggtttcgccacctctgacttgagcgtcgatttttgtgatgctcgtca |
| | | ggggggcggagcctatggaaaaacgccagcaacgcggcctttttacggttcctggccttttgctggcc |
| | | ttttgctcacatgttctttcctgcgttatcccctgattctgtggataaccgtattaccgcc |
| 11 | plasmid | atgcattagttattaatagtaatcaattacggggtcattagttcatagcccatatatggagttccgcgttaca |
| | pCP4-H2 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggtaccaagcttcgaggcaggtgg |
| | | acagcttggttctaatgaagttaaccctgtcgttctgcgacatctgggcccggaaagcgtttaactgatg |
| | | gatggaacagtccttctgcggtaccaggtaagtgtacccaattcgccctatagtgagtcgtattacaattc |
| | | actcgatcgcccttcccaacagttgcgcagcctgaatggcgaatggagatccaatttttaagtgtataat |
| | | gtgttaaactactgattctaattgtttgtgtattttagattcacagtcccaaggctcatttcaggcccctcagt |
| | | cctcacagtctgttcatgatcataatcagccataccacatttgtagaggttttacttgctttaaaaaacctcc |
| | | cacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtt |
| | | tgtccaaactcatcaatgtatcttaacgcgtttaccaatgcttaatcagtgaggcacctatctcagcgatct |
| | | gtctatttcgttcatccatagttgcctgactccccgtcgtgtagataactacgatacgggagggcttacca |
| | | tctggccccagtgctgcaatgataccgcgagacccacgctcaccggctccagatttatcagcaataaa |
| | | ccagccagccggaagggccgagcgcagaagtggtcctgcaactttatccgcctccatccagtctatta |
| | | attgttgccgggaagctagagtaagtagttcgccagttaatagtttgcgcaacgttgttgccattgctaca |
| | | ggcatcgtggtgtcacgctcgtcgtttggtatggcttcattcagctccggttcccaacgatcaaggcgag |
| | | ttacatgatcccccatgttgtgcaaaaaagcggttagctccttcggtcctccgatcgttgtcagaagtaag |
| | | ttggccgcagtgttatcactcatggttatggcagcactgcataattctcttactgtcatgccatccgtaaga |
| | | tgcttttctgtgactggtgagtactcaaccaagtcattctgagaatagtgtatgcggcgaccgagttgctc |
| | | ttgcccggcgtcaatacgggataataccgcgccacatagcagaactttaaaagtgctcatcattggaaa |
| | | acgttcttcggggcgaaaactctcaaggatcttaccgctgttgagatccagttcgatgtaacccactcgt |
| | | gcacccaactgatcttcagcatcttttactttcaccagcgtttctgggtgagcaaaaacaggaaggcaaa |
| | | atgccgcaaaaaagggaataagggcgacacggaaatgttgaatactcatactcttcctttttcaatattat |
| | | tgaagcatttatcagggttattgtctcatgagcggatacatatttgaatgtatttagaaaaataaacaaata |
| | | ggggtacgcgtaaattgtaagcgttaatattttgttaaaattcgcgttaaatttttgttaaatcagctcatttttt |
| | | aaccaataggccgaaatcggcaaaatcccttataaatcaaaagaatagaccgagatagggttgagtgtt |
| | | gttccagtttggaacaagagtccactattaaagaacgtggactccaacgtcaaagggcgaaaaaccgt |
| | | ctatcagggcgatggcccactacgtgaaccatcaccctaatcaagttttttggggtcgaggtgccgtaa |
| | | agcactaaatcggaaccctaaagggagcccccgatttagagcttgacggggaaagccggcgaacgt |
| | | ggcgagaaaggaagggaagaaagcgaaaggagcgggcgctagggcgctggcaagtgtagcggt |
| | | cacgctgcgcgtaaccaccacacccgccgcgcttaatgcgccgctacagggcgcgtcaggtggcac |
| | | ttttcggggaaatgtgcgcggaacccctatttgtttatttttctaaatacattcaaatatgtatccgctcatga |
| | | gacaataaccctgataaatgcttcaataatattgaaaaaggaagaatcctgaggcggaaagaaccagc |
| | | tgtggaatgtgtgtcagttagggtgtggaaagtccccaggctccccagcaggcagaagtatgcaaagc |
| | | atgcatctcaattagtcagcaaccaggtgtggaaagtccccaggctccccagcaggcagaagtatgca |
| | | aagcatgcatctcaattagtcagcaaccatagtcccgcccctaactccgcccatcccgcccctaactcc |
| | | gcccagttccgcccattctccgccccatggctgactaattttttttatttatgcagaggccgaggccgcct |
| | | cggcctctgagctattccagaagtagtgaggaggcttttttggaggcctaggcttttgcaaatatcgatat |
| | | gaaaaagcctgaactcaccgcgacgtctgtcgagaagtttctgatcgaaaagttcgacagcgtctccg |
| | | acctgatgcagctctcggagggcgaagaatctcgtgctttcagcttcgatgtaggagggcgtggatatg |
| | | tcctgcgggtaaatagctgcgccgatggtttctacaaagatcgttatgtttatcggcactttgcatcggcc |
| | | gcgctcccgattccggaagtgcttgacattggggaattcagcgagagcctgacctattgcatctcccgc |
| | | cgtgcacagggtgtcacgttgcaagacctgcctgaaaccgaactgcccgctgttctgcagccggtcgc |
| | | ggaggccatggatgcgatcgctgcggccgatcttagccagacgagcgggttcggcccattcggacc |
| | | gcaaggaatcggtcaatacactacatggcgtgatttcatatgcgcgattgctgatccccatgtgtatcact |
| | | ggcaaactgtgatggacgacaccgtcagtgcgtccgtcgcgcaggctctcgatgagctgatgctttgg |
| | | gccgaggactgccccgaagtccggcacctcgtgcacgcggatttcggctccaacaatgtcctgacgg |
| | | acaatggccgcataacagcggtcattgactggagcgaggcgatgttcggggattcccaatacgaggt |
| | | cgccaacatcttcttctggaggccgtggttggcttgtatggagcagcagacgcgctacttcgagcgga |
| | | ggcatccggagcttgcaggatcgccgcggctccgggcgtatatgctccgcattggtcttgaccaactct |
| | | atcagagcttggttgacggcaatttcgatgatgcagcttgggcgcagggtcgatgcgacgcaatcgtc |
| | | cgatccggagccgggactgtcgggcgtacacaaatcgcccgcagaagcgcggccgtctggaccga |
| | | tggctgtgtagaagtactcgccgatagtggaaaccgacgccccagcactcgtccgagggcaaagga |
| | | atagtgagcgggactctggggttcgaaatgaccgaccaagcgacgcccaacctgccatcacgagatt |
| | | tcgattccaccgccgccttctatgaaaggttgggcttcggaatcgttttccgggacgccggctggatgat |
| | | cctccagcgcggggatctcatgctggagttcttcgcccaccctagggggaggctaactgaaacacgg |
| | | aaggagacaataccggaaggaacccgcgctatgacggcaataaaaagacagaataaaacgcacgg |
| | | tgttgggtcgtttgttcataaacgcggggttcggtcccagggctggcactctgtcgataccccaccgag |
| | | accccattggggccaatacgcccgcgtttcttccttttccccaccccaccccccaagttcgggtgaagg |
| | | cccagggctcgcagccaacgtcggggcggcaggccctgccatagcctcaggttactcatatatacttt |
| | | agattgatttaaaacttcatttttaatttaaaaggatctaggtgaagatcctttttgataatctcatgaccaaa |
| | | atcccttaacgtgagttttcgttccactgagcgtcagaccccgtagaaaagatcaaaggatcttcttgag |
| | | atcctttttttctgcgcgtaatctgctgcttgcaaacaaaaaaaccaccgctaccagcggtggtttgtttgc |
| | | cggatcaagagctaccaactctttttccgaaggtaactggcttcagcagagcgcagataccaaatactg |
| | | tccttctagtgtagccgtagttaggccaccacttcaagaactctgtagcaccgcctacatacctcgctctg |
| | | ctaatcctgttaccagtggctgctgccagtggcgataagtcgtgtcttaccgggttggactcaagacgat |
| | | agttaccggataaggcgcagcggtcgggctgaacggggggttcgtgcacacagcccagcttggagc |
| | | gaacgacctacaccgaactgagatacctacagcgtgagctatgagaaagcgccacgcttcccgaag |
| | | ggagaaaggcggacaggtatccggtaagcggcagggtcggaacaggagagcgcacgagggagc |
| | | ttccagggggaaacgcctggtatctttatagtcctgtcgggtttcgccacctctgacttgagcgtcgatttt |
| | | tgtgatgctcgtcaggggggcggagcctatggaaaaacgccagcaacgcggcctttttacggttcctg |
| | | gccttttgctggccttttgctcacatgttctttcctgcgttatcccctgattctgtggataaccgtattaccgc |
| | | c |
| 12 | plasmid | atgcattagttattaatagtaatcaattacggggtcattagttcatagcccatatatggagttccgcgttaca |
| | pCP4-H3 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaaggg |
| | | cgaattcggatccgcggccgccttaagctcgaggcaggtggacaggaaggttctaatgttctataggg |
| | | tctgcttgtcgctcatctgggcccggagatgcgtaaagtcagacatccggtacagtccttctgcggtacc |
| | | aggtaagtgtacccaattcgccctatagtgagtcgtattacaattcactcgatcgcccttcccaacagttg |
| | | cgcagcctgaatggcgaatggagatccaatttttaagtgtataatgtgttaaactactgattctaattgtttg |
| | | tgtattttagattcacagtcccaaggctcatttcaggcccctcagtcctcacagtctgttcatgatcataatc |
| | | agccataccacatttgtagaggttttacttgctttaaaaaacctcccacacctccccctgaacctgaaaca |
| | | taaaatgaatgcaattgttgttgttaacttgtttattgcagcttataatggttacaaataaagcaatagcatca |
| | | caaatttcacaaataaagcatttttttcactgcattctagttgtggtttgtccaaactcatcaatgtatcttaac |
| | | gcgtttaccaatgcttaatcagtgaggcacctatctcagcgatctgtctatttcgttcatccatagttgcctg |
| | | actccccgtcgtgtagataactacgatacgggagggcttaccatctggccccagtgctgcaatgatacc |
| | | gcgagacccacgctcaccggctccagatttatcagcaataaaccagccagccggaagggccgagcg |
| | | cagaagtggtcctgcaactttatccgcctccatccagtctattaattgttgccgggaagctagagtaagta |
| | | gttcgccagttaatagtttgcgcaacgttgttgccattgctacaggcatcgtggtgtcacgctcgtcgtttg |
| | | gtatggcttcattcagctccggttcccaacgatcaaggcgagttacatgatcccccatgttgtgcaaaaa |
| | | agcggttagctccttcggtcctccgatcgttgtcagaagtaagttggccgcagtgttatcactcatggtta |
| | | tggcagcactgcataattctcttactgtcatgccatccgtaagatgcttttctgtgactggtgagtactcaa |
| | | ccaagtcattctgagaatagtgtatgcggcgaccgagttgctcttgcccggcgtcaatacgggataata |
| | | ccgcgccacatagcagaactttaaaagtgctcatcattggaaaacgttcttcggggcgaaaactctcaa |
| | | ggatcttaccgctgttgagatccagttcgatgtaacccactcgtgcacccaactgatcttcagcatctttta |
| | | ctttcaccagcgtttctgggtgagcaaaaacaggaaggcaaaatgccgcaaaaaagggaataagggc |
| | | gacacggaaatgttgaatactcatactcttcctttttcaatattattgaagcatttatcagggttattgtctcat |
| | | gagcggatacatatttgaatgtatttagaaaaataaacaaataggggtacgcgtaaattgtaagcgttaat |
| | | attttgttaaaattcgcgttaaatttttgttaaatcagctcattttttaaccaataggccgaaatcggcaaaat |
| | | cccttataaatcaaaagaatagaccgagatagggttgagtgttgttccagtttggaacaagagtccacta |
| | | ttaaagaacgtggactccaacgtcaaagggcgaaaaaccgtctatcagggcgatggcccactacgtg |
| | | aaccatcaccctaatcaagttttttggggtcgaggtgccgtaaagcactaaatcggaaccctaaaggga |
| | | gcccccgatttagagcttgacggggaaagccggcgaacgtggcgagaaaggaagggaagaaagc |
| | | gaaaggagcgggcgctagggcgctggcaagtgtagcggtcacgctgcgcgtaaccaccacacccg |
| | | ccgcgcttaatgcgccgctacagggcgcgtcaggtggcacttttcggggaaatgtgcgcggaacccc |
| | | tatttgtttatttttctaaatacattcaaatatgtatccgctcatgagacaataaccctgataaatgcttcaata |
| | | atattgaaaaaggaagaatcctgaggcggaaagaaccagctgtggaatgtgtgtcagttagggtgtgg |
| | | aaagtccccaggctccccagcaggcagaagtatgcaaagcatgcatctcaattagtcagcaaccagg |
| | | tgtggaaagtccccaggctccccagcaggcagaagtatgcaaagcatgcatctcaattagtcagcaac |
| | | catagtcccgcccctaactccgcccatcccgcccctaactccgcccagttccgcccattctccgcccca |
| | | tggctgactaattttttttatttatgcagaggccgaggccgcctcggcctctgagctattccagaagtagt |
| | | gaggaggcttttttggaggcctaggcttttgcaaatatcgatatgaaaaagcctgaactcaccgcgacgt |
| | | ctgtcgagaagtttctgatcgaaaagttcgacagcgtctccgacctgatgcagctctcggagggcgaa |
| | | gaatctcgtgctttcagcttcgatgtaggagggcgtggatatgtcctgcgggtaaatagctgcgccgat |
| | | ggtttctacaaagatcgttatgtttatcggcactttgcatcggccgcgctcccgattccggaagtgcttga |
| | | cattggggaattcagcgagagcctgacctattgcatctcccgccgtgcacagggtgtcacgttgcaag |
| | | acctgcctgaaaccgaactgcccgctgttctgcagccggtcgcggaggccatggatgcgatcgctgc |
| | | ggccgatcttagccagacgagcgggttcggcccattcggaccgcaaggaatcggtcaatacactaca |
| | | tggcgtgatttcatatgcgcgattgctgatccccatgtgtatcactggcaaactgtgatggacgacaccg |
| | | tcagtgcgtccgtcgcgcaggctctcgatgagctgatgctttgggccgaggactgccccgaagtccg |
| | | gcacctcgtgcacgcggatttcggctccaacaatgtcctgacggacaatggccgcataacagcggtc |
| | | attgactggagcgaggcgatgttcggggattcccaatacgaggtcgccaacatcttcttctggaggcc |
| | | gtggttggcttgtatggagcagcagacgcgctacttcgagcggaggcatccggagcttgcaggatcg |
| | | ccgcggctccgggcgtatatgctccgcattggtcttgaccaactctatcagagcttggttgacggcaatt |
| | | tcgatgatgcagcttgggcgcagggtcgatgcgacgcaatcgtccgatccggagccgggactgtcg |
| | | ggcgtacacaaatcgcccgcagaagcgcggccgtctggaccgatggctgtgtagaagtactcgccg |
| | | atagtggaaaccgacgccccagcactcgtccgagggcaaaggaatagtgagcgggactctggggtt |
| | | cgaaatgaccgaccaagcgacgcccaacctgccatcacgagatttcgattccaccgccgccttctatg |
| | | aaaggttgggcttcggaatcgttttccgggacgccggctggatgatcctccagcgcggggatctcatg |
| | | ctggagttcttcgcccaccctagggggaggctaactgaaacacggaaggagacaataccggaagga |
| | | acccgcgctatgacggcaataaaaagacagaataaaacgcacggtgttgggtcgtttgttcataaacg |
| | | cggggttcggtcccagggctggcactctgtcgataccccaccgagaccccattggggccaatacgcc |
| | | cgcgtttcttccttttccccaccccaccccccaagttcgggtgaaggcccagggctcgcagccaacgtc |
| | | ggggcggcaggccctgccatagcctcaggttactcatatatactttagattgatttaaaacttcatttttaat |
| | | ttaaaaggatctaggtgaagatcctttttgataatctcatgaccaaaatcccttaacgtgagttttcgttcca |
| | | ctgagcgtcagaccccgtagaaaagatcaaaggatcttcttgagatcctttttttctgcgcgtaatctgct |
| | | gcttgcaaacaaaaaaaccaccgctaccagcggtggtttgtttgccggatcaagagctaccaactctttt |
| | | tccgaaggtaactggcttcagcagagcgcagataccaaatactgtccttctagtgtagccgtagttagg |
| | | ccaccacttcaagaactctgtagcaccgcctacatacctcgctctgctaatcctgttaccagtggctgct |
| | | gccagtggcgataagtcgtgtcttaccgggttggactcaagacgatagttaccggataaggcgcagcg |
| | | gtcgggctgaacggggggttcgtgcacacagcccagcttggagcgaacgacctacaccgaactgag |
| | | atacctacagcgtgagctatgagaaagcgccacgcttcccgaagggagaaaggcggacaggtatcc |
| | | ggtaagcggcagggtcggaacaggagagcgcacgagggagcttccagggggaaacgcctggtat |
| | | ctttatagtcctgtcgggtttcgccacctctgacttgagcgtcgatttttgtgatgctcgtcaggggggcg |
| | | gagcctatggaaaaacgccagcaacgcggcctttttacggttcctggccttttgctggccttttgctcaca |
| | | tgttctttcctgcgttatcccctgattctgtggataaccgtattaccgcc |
| 13 | plasmid | atgcattagttattaatagtaatcaattacggggtcattagttcatagcccatatatggagttccgcgttaca |
| | pCP4-Z | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaccgg |
| | | tggtaccaggtaagtgtacccaattcgccctatagtgagtcgtattacaattcactcgatcgcccttccca |
| | | acagttgcgcagcctgaatggcgaatggagatccaatttttaagtgtataatgtgttaaactactgattcta |
| | | attgtttgtgtattttagattcacagtcccaaggctcatttcaggcccctcagtcctcacagtctgttcatgat |
| | | cataatcagccataccacatttgtagaggttttacttgctttaaaaaacctcccacacctccccctgaacct |
| | | gaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgt |
| | | atcttaacgcgtttaccaatgcttaatcagtgaggcacctatctcagcgatctgtctatttcgttcatccata |
| | | gttgcctgactccccgtcgtgtagataactacgatacgggagggcttaccatctggccccagtgctgca |
| | | atgataccgcgagacccacgctcaccggctccagatttatcagcaataaaccagccagccggaaggg |
| | | ccgagcgcagaagtggtcctgcaactttatccgcctccatccagtctattaattgttgccgggaagctag |
| | | agtaagtagttcgccagttaatagtttgcgcaacgttgttgccattgctacaggcatcgtggtgtcacgct |
| | | cgtcgtttggtatggcttcattcagctccggttcccaacgatcaaggcgagttacatgatcccccatgttg |
| | | tgcaaaaaagcggttagctccttcggtcctccgatcgttgtcagaagtaagttggccgcagtgttatcac |
| | | tcatggttatggcagcactgcataattctcttactgtcatgccatccgtaagatgcttttctgtgactggtga |
| | | gtactcaaccaagtcattctgagaatagtgtatgcggcgaccgagttgctcttgcccggcgtcaatacg |
| | | ggataataccgcgccacatagcagaactttaaaagtgctcatcattggaaaacgttcttcggggcgaaa |
| | | actctcaaggatcttaccgctgttgagatccagttcgatgtaacccactcgtgcacccaactgatcttcag |
| | | catcttttactttcaccagcgtttctgggtgagcaaaaacaggaaggcaaaatgccgcaaaaaaggga |
| | | ataagggcgacacggaaatgttgaatactcatactcttcctttttcaatattattgaagcatttatcagggtt |
| | | attgtctcatgagcggatacatatttgaatgtatttagaaaaataaacaaataggggtacgcgtaaattgta |
| | | agcgttaatattttgttaaaattcgcgttaaatttttgttaaatcagctcattttttaaccaataggccgaaatc |
| | | ggcaaaatcccttataaatcaaaagaatagaccgagatagggttgagtgttgttccagtttggaacaag |
| | | agtccactattaaagaacgtggactccaacgtcaaagggcgaaaaaccgtctatcagggcgatggcc |
| | | cactacgtgaaccatcaccctaatcaagttttttggggtcgaggtgccgtaaagcactaaatcggaacc |
| | | ctaaagggagcccccgatttagagcttgacggggaaagccggcgaacgtggcgagaaaggaaggg |
| | | aagaaagcgaaaggagcgggcgctagggcgctggcaagtgtagcggtcacgctgcgcgtaaccac |
| | | cacacccgccgcgcttaatgcgccgctacagggcgcgtcaggtggcacttttcggggaaatgtgcgc |
| | | ggaacccctatttgtttatttttctaaatacattcaaatatgtatccgctcatgagacaataaccctgataaat |
| | | gcttcaataatattgaaaaaggaagaatcctgaggcggaaagaaccagctgtggaatgtgtgtcagtta |
| | | gggtgtggaaagtccccaggctccccagcaggcagaagtatgcaaagcatgcatctcaattagtcag |
| | | caaccaggtgtggaaagtccccaggctccccagcaggcagaagtatgcaaagcatgcatctcaatta |
| | | gtcagcaaccatagtcccgcccctaactccgcccatcccgcccctaactccgcccagttccgcccattc |
| | | tccgccccatggctgactaattttttttatttatgcagaggccgaggccgcctcggcctctgagctattcc |
| | | agaagtagtgaggaggcttttttggaggcctaggcttttgcaaatatcgatatggccaagttgaccagtg |
| | | ccgttccggtgctcaccgcgcgcgacgtcgccggagcggtcgagttctggaccgaccggctcgggtt |
| | | ctcccgggacttcgtggaggacgacttcgccggtgtggtccgggacgacgtgaccctgttcatcagc |
| | | gcggtccaggaccaggtggtgccggacaacaccctggcctgggtgtgggtgcgcggcctggacga |
| | | gctgtacgccgagtggtcggaggtcgtgtccacgaacttccgggacgcctccgggccggccatgac |
| | | cgagatcggcgagcagccgtggggggggagttcgccctgcgcgacccggccggcaactgcgtg |
| | | cacttcgtggccgaggagcaggactgatgagcgggactctggggttcgaaatgaccgaccaagcga |
| | | cgcccaacctgccatcacgagatttcgattccaccgccgccttctatgaaaggttgggcttcggaatcgt |
| | | tttccgggacgccggctggatgatcctccagcgcggggatctcatgctggagttcttcgcccaccctag |
| | | ggggaggctaactgaaacacggaaggagacaataccggaaggaacccgcgctatgacggcaataa |
| | | aaagacagaataaaacgcacggtgttgggtcgtttgttcataaacgcggggttcggtcccagggctgg |
| | | cactctgtcgataccccaccgagaccccattggggccaatacgcccgcgtttcttccttttccccacccc |
| | | accccccaagttcgggtgaaggcccagggctcgcagccaacgtcggggcggcaggccctgccata |
| | | gcctcaggttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtagaa |
| | | aagatcaaaggatcttcttgagatcctttttttctgcgcgtaatctgctgcttgcaaacaaaaaaaccaccg |
| | | ctaccagcggtggtttgtttgccggatcaagagctaccaactctttttccgaaggtaactggcttcagca |
| | | gagcgcagataccaaatactgtccttctagtgtagccgtagttaggccaccacttcaagaactctgtagc |
| | | accgcctacatacctcgctctgctaatcctgttaccagtggctgctgccagtggcgataagtcgtgtctta |
| | | ccgggttggactcaagacgatagttaccggataaggcgcagcggtcgggctgaacggggggttcgt |
| | | gcacacagcccagcttggagcgaacgacctacaccgaactgagatacctacagcgtgagctatgag |
| | | aaagcgccacgcttcccgaagggagaaaggcggacaggtatccggtaagcggcagggtcggaac |
| | | aggagagcgcacgagggagcttccagggggaaacgcctggtatctttatagtcctgtcgggtttcgcc |
| | | acctctgacttgagcgtcgatttttgtgatgctcgtcaggggggggagcctatggaaaaacgccagc |
| | | aacgcggcctttttacggttcctggccttttgctggccttttgctcacatgttctttcctgcgttatcccctgat |
| | | tctgtggataaccgtattaccgcc |
| 14 | plasmid | atgcattagttattaatagtaatcaattacggggtcattagttcatagcccatatatggagttccgcgttaca |
| | pCP4-Z1 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggaggcaggtggacaggaaggttc |
| | | taatgttcttaaggcacaggaactgggacatctgggcccggaaagcctttttctctgtgatccggtacag |
| | | tccttctgcggtaccaggtaagtgtacccaattcgccctatagtgagtcgtattacaattcactcgatcgc |
| | | ccttcccaacagttgcgcagcctgaatggcgaatggagatccaatttttaagtgtataatgtgttaaacta |
| | | ctgattctaattgtttgtgtattttagattcacagtcccaaggctcatttcaggcccctcagtcctcacagtct |
| | | gttcatgatcataatcagccataccacatttgtagaggttttacttgctttaaaaaacctcccacacctccc |
| | | cctgaacctgaaacataaaatgaatgcaattgttgttgttaacttgtttattgcagcttataatggttacaaat |
| | | aaagcaatagcatcacaaatttcacaaataaagcatttttttcactgcattctagttgtggtttgtccaaact |
| | | catcaatgtatcttaacgcgtttaccaatgcttaatcagtgaggcacctatctcagcgatctgtctatttcgt |
| | | tcatccatagttgcctgactccccgtcgtgtagataactacgatacgggagggcttaccatctggcccca |
| | | gtgctgcaatgataccgcgagacccacgctcaccggctccagatttatcagcaataaaccagccagcc |
| | | ggaagggccgagcgcagaagtggtcctgcaactttatccgcctccatccagtctattaattgttgccgg |
| | | gaagctagagtaagtagttcgccagttaatagtttgcgcaacgttgttgccattgctacaggcatcgtggt |
| | | gtcacgctcgtcgtttggtatggcttcattcagctccggttcccaacgatcaaggcgagttacatgatccc |
| | | ccatgttgtgcaaaaaagcggttagctccttcggtcctccgatcgttgtcagaagtaagttggccgcagt |
| | | gttatcactcatggttatggcagcactgcataattctcttactgtcatgccatccgtaagatgcttttctgtga |
| | | ctggtgagtactcaaccaagtcattctgagaatagtgtatgcggcgaccgagttgctcttgcccggcgt |
| | | caatacgggataataccgcgccacatagcagaactttaaaagtgctcatcattggaaaacgttcttcgg |
| | | ggcgaaaactctcaaggatcttaccgctgttgagatccagttcgatgtaacccactcgtgcacccaact |
| | | gatcttcagcatcttttactttcaccagcgtttctgggtgagcaaaaacaggaaggcaaaatgccgcaaa |
| | | aaagggaataagggcgacacggaaatgttgaatactcatactcttcctttttcaatattattgaagcatttat |
| | | cagggttattgtctcatgagcggatacatatttgaatgtatttagaaaaataaacaaataggggtacgcgt |
| | | aaattgtaagcgttaatattttgttaaaattcgcgttaaatttttgttaaatcagctcattttttaaccaataggc |
| | | cgaaatcggcaaaatcccttataaatcaaaagaatagaccgagatagggttgagtgttgttccagtttgg |
| | | aacaagagtccactattaaagaacgtggactccaacgtcaaagggcgaaaaaccgtctatcagggcg |
| | | atggcccactacgtgaaccatcaccctaatcaagttttttggggtcgaggtgccgtaaagcactaaatcg |
| | | gaaccctaaagggagcccccgatttagagcttgacggggaaagccggcgaacgtggcgagaaagg |
| | | aagggaagaaagcgaaaggagcgggcgctagggcgctggcaagtgtagcggtcacgctgcgcgt |
| | | aaccaccacacccgccgcgcttaatgcgccgctacagggcgcgtcaggtggcacttttcggggaaat |
| | | gtgcgcggaacccctatttgtttatttttctaaatacattcaaatatgtatccgctcatgagacaataaccct |
| | | gataaatgcttcaataatattgaaaaaggaagaatcctgaggcggaaagaaccagctgtggaatgtgt |
| | | gtcagttagggtgtggaaagtccccaggctccccagcaggcagaagtatgcaaagcatgcatctcaat |
| | | tagtcagcaaccaggtgtggaaagtccccaggctccccagcaggcagaagtatgcaaagcatgcatc |
| | | tcaattagtcagcaaccatagtcccgcccctaactccgcccatcccgcccctaactccgcccagttccg |
| | | cccattctccgccccatggctgactaattttttttatttatgcagaggccgaggccgcctcggcctctgag |
| | | ctattccagaagtagtgaggaggcttttttggaggcctaggcttttgcaaatatcgatatggccaagttga |
| | | ccagtgccgttccggtgctcaccgcgcgcgacgtcgccggagcggtcgagttctggaccgaccggc |
| | | tcgggttctcccgggacttcgtggaggacgacttcgccggtgtggtccgggacgacgtgaccctgttc |
| | | atcagcgcggtccaggaccaggtggtgccggacaacaccctggcctgggtgtgggtgcgcggcctg |
| | | gacgagctgtacgccgagtggtcggaggtcgtgtccacgaacttccgggacgcctccgggccggcc |
| | | atgaccgagatcggcgagcagccgtggggggggagttcgccctgcgcgacccggccggcaact |
| | | gcgtgcacttcgtggccgaggagcaggactgatgagcgggactctggggttcgaaatgaccgacca |
| | | agcgacgcccaacctgccatcacgagatttcgattccaccgccgccttctatgaaaggttgggcttcgg |
| | | aatcgttttccgggacgccggctggatgatcctccagcgcggggatctcatgctggagttcttcgccca |
| | | ccctagggggaggctaactgaaacacggaaggagacaataccggaaggaacccgcgctatgacgg |
| | | caataaaaagacagaataaaacgcacggtgttgggtcgtttgttcataaacgcggggttcggtcccag |
| | | ggctggcactctgtcgataccccaccgagaccccattggggccaatacgcccgcgtttcttccttttccc |
| | | caccccaccccccaagttcgggtgaaggcccagggctcgcagccaacgtcggggcggcaggccct |
| | | gccatagcctcaggttactcatatatactttagattgatttaaaacttcatttttaatttaaaaggatctaggtg |
| | | aagatcctttttgataatctcatgaccaaaatcccttaacgtgagttttcgttccactgagcgtcagacccc |
| | | gtagaaaagatcaaaggatcttcttgagatcctttttttctgcgcgtaatctgctgcttgcaaacaaaaaaa |
| | | ccaccgctaccagcggtggtttgtttgccggatcaagagctaccaactctttttccgaaggtaactggctt |
| | | cagcagagcgcagataccaaatactgtccttctagtgtagccgtagttaggccaccacttcaagaactc |
| | | tgtagcaccgcctacatacctcgctctgctaatcctgttaccagtggctgctgccagtggcgataagtcg |
| | | tgtcttaccgggttggactcaagacgatagttaccggataaggcgcagcggtcgggctgaacggggg |
| | | gttcgtgcacacagcccagcttggagcgaacgacctacaccgaactgagatacctacagcgtgagct |
| | | atgagaaagcgccacgcttcccgaagggagaaaggcggacaggtatccggtaagcggcagggtcg |
| | | gaacaggagagcgcacgagggagcttccagggggaaacgcctggtatctttatagtcctgtcgggttt |
| | | cgccacctctgacttgagcgtcgatttttgtgatgctcgtcaggggggcggagcctatggaaaaacgc |
| | | cagcaacgcggcctttttacggttcctggccttttgctggccttttgctcacatgttctttcctgcgttatccc |
| | | ctgattctgtggataaccgtattaccgcc |
| 15 | plasmid | atgcattagttattaatagtaatcaattacggggtcattagttcatagcccatatatggagttccgcgttaca |
| | pCP4-Z2 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggtaccaagcttcgaggcaggtgg |
| | | acagcttggttctaatgaagttaaccctgtcgttctgcgacatctgggcccggaaagcgtttaactgatg |
| | | gatggaacagtccttctgcggtaccaggtaagtgtacccaattcgccctatagtgagtcgtattacaattc |
| | | actcgatcgcccttcccaacagttgcgcagcctgaatggcgaatggagatccaatttttaagtgtataat |
| | | gtgttaaactactgattctaattgtttgtgtattttagattcacagtcccaaggctcatttcaggcccctcagt |
| | | cctcacagtctgttcatgatcataatcagccataccacatttgtagaggttttacttgctttaaaaaacctcc |
| | | cacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtt |
| | | tgtccaaactcatcaatgtatcttaacgcgtttaccaatgcttaatcagtgaggcacctatctcagcgatct |
| | | gtctatttcgttcatccatagttgcctgactccccgtcgtgtagataactacgatacgggagggcttacca |
| | | tctggccccagtgctgcaatgataccgcgagacccacgctcaccggctccagatttatcagcaataaa |
| | | ccagccagccggaagggccgagcgcagaagtggtcctgcaactttatccgcctccatccagtctatta |
| | | attgttgccgggaagctagagtaagtagttcgccagttaatagtttgcgcaacgttgttgccattgctaca |
| | | ggcatcgtggtgtcacgctcgtcgtttggtatggcttcattcagctccggttcccaacgatcaaggcgag |
| | | ttacatgatcccccatgttgtgcaaaaaagcggttagctccttcggtcctccgatcgttgtcagaagtaag |
| | | ttggccgcagtgttatcactcatggttatggcagcactgcataattctcttactgtcatgccatccgtaaga |
| | | tgcttttctgtgactggtgagtactcaaccaagtcattctgagaatagtgtatgcggcgaccgagttgctc |
| | | ttgcccggcgtcaatacgggataataccgcgccacatagcagaactttaaaagtgctcatcattggaaa |
| | | acgttcttcggggcgaaaactctcaaggatcttaccgctgttgagatccagttcgatgtaacccactcgt |
| | | gcacccaactgatcttcagcatcttttactttcaccagcgtttctgggtgagcaaaaacaggaaggcaaa |
| | | atgccgcaaaaaagggaataagggcgacacggaaatgttgaatactcatactcttcctttttcaatattat |
| | | tgaagcatttatcagggttattgtctcatgagcggatacatatttgaatgtatttagaaaaataaacaaata |
| | | ggggtacgcgtaaattgtaagcgttaatattttgttaaaattcgcgttaaatttttgttaaatcagctcatttttt |
| | | aaccaataggccgaaatcggcaaaatcccttataaatcaaaagaatagaccgagatagggttgagtgtt |
| | | gttccagtttggaacaagagtccactattaaagaacgtggactccaacgtcaaagggcgaaaaaccgt |
| | | ctatcagggcgatggcccactacgtgaaccatcaccctaatcaagttttttggggtcgaggtgccgtaa |
| | | agcactaaatcggaaccctaaagggagcccccgatttagagcttgacggggaaagccggcgaacgt |
| | | ggcgagaaaggaagggaagaaagcgaaaggagcgggcgctagggcgctggcaagtgtagcggt |
| | | cacgctgcgcgtaaccaccacacccgccgcgcttaatgcgccgctacagggcgcgtcaggtggcac |
| | | ttttcggggaaatgtgcgcggaacccctatttgtttatttttctaaatacattcaaatatgtatccgctcatga |
| | | gacaataaccctgataaatgcttcaataatattgaaaaaggaagaatcctgaggcggaaagaaccagc |
| | | tgtggaatgtgtgtcagttagggtgtggaaagtccccaggctccccagcaggcagaagtatgcaaagc |
| | | atgcatctcaattagtcagcaaccaggtgtggaaagtccccaggctccccagcaggcagaagtatgca |
| | | aagcatgcatctcaattagtcagcaaccatagtcccgcccctaactccgcccatcccgcccctaactcc |
| | | gcccagttccgcccattctccgccccatggctgactaattttttttatttatgcagaggccgaggccgcct |
| | | cggcctctgagctattccagaagtagtgaggaggcttttttggaggcctaggcttttgcaaatatcgatat |
| | | ggccaagttgaccagtgccgttccggtgctcaccgcgcgcgacgtcgccggagcggtcgagttctgg |
| | | accgaccggctcgggttctcccgggacttcgtggaggacgacttcgccggtgtggtccgggacgac |
| | | gtgaccctgttcatcagcgcggtccaggaccaggtggtgccggacaacaccctggcctgggtgtggg |
| | | tgcgcggcctggacgagctgtacgccgagtggtcggaggtcgtgtccacgaacttccgggacgcctc |
| | | cgggccggccatgaccgagatcggcgagcagccgtggggggggagttcgccctgcgcgacccg |
| | | gccggcaactgcgtgcacttcgtggccgaggagcaggactgatgagcgggactctggggttcgaaa |
| | | tgaccgaccaagcgacgcccaacctgccatcacgagatttcgattccaccgccgccttctatgaaagg |
| | | ttgggcttcggaatcgttttccgggacgccggctggatgatcctccagcgcggggatctcatgctgga |
| | | gttcttcgcccaccctagggggaggctaactgaaacacggaaggagacaataccggaaggaacccg |
| | | cgctatgacggcaataaaaagacagaataaaacgcacggtgttgggtcgtttgttcataaacgcgggg |
| | | ttcggtcccagggctggcactctgtcgataccccaccgagaccccattggggccaatacgcccgcgtt |
| | | tcttccttttccccaccccaccccccaagttcgggtgaaggcccagggctcgcagccaacgtcggggc |
| | | ggcaggccctgccatagcctcaggttactcatatatactttagattgatttaaaacttcatttttaatttaaaa |
| | | ggatctaggtgaagatcctttttgataatctcatgaccaaaatcccttaacgtgagttttcgttccactgag |
| | | cgtcagaccccgtagaaaagatcaaaggatcttcttgagatcctttttttctgcgcgtaatctgctgcttgc |
| | | aaacaaaaaaaccaccgctaccagcggtggtttgtttgccggatcaagagctaccaactctttttccgaa |
| | | ggtaactggcttcagcagagcgcagataccaaatactgtccttctagtgtagccgtagttaggccacca |
| | | cttcaagaactctgtagcaccgcctacatacctcgctctgctaatcctgttaccagtggctgctgccagtg |
| | | gcgataagtcgtgtcttaccgggttggactcaagacgatagttaccggataaggcgcagcggtcggg |
| | | ctgaacggggggttcgtgcacacagcccagcttggagcgaacgacctacaccgaactgagataccta |
| | | cagcgtgagctatgagaaagcgccacgcttcccgaagggagaaaggcggacaggtatccggtaag |
| | | cggcagggtcggaacaggagagcgcacgagggagcttccagggggaaacgcctggtatctttatag |
| | | tcctgtcgggtttcgccacctctgacttgagcgtcgatttttgtgatgctcgtcaggggggcggagcctat |
| | | ggaaaaacgccagcaacgcggcctttttacggttcctggccttttgctggccttttgctcacatgttctttc |
| | | ctgcgttatcccctgattctgtggataaccgtattaccgcc |
| 16 | plasmid | atgcattagttattaatagtaatcaattacggggtcattagttcatagcccatatatggagttccgcgttaca |
| | pCP4-Z3 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaaggg |
| | | cgaattcggatccgcggccgccttaagctcgaggcaggtggacaggaaggttctaatgttctataggg |
| | | tctgcttgtcgctcatctgggcccggagatgcgtaaagtcagacatccggtacagtccttctgcggtacc |
| | | aggtaagtgtacccaattcgccctatagtgagtcgtattacaattcactcgatcgcccttcccaacagttg |
| | | cgcagcctgaatggcgaatggagatccaatttttaagtgtataatgtgttaaactactgattctaattgtttg |
| | | tgtattttagattcacagtcccaaggctcatttcaggcccctcagtcctcacagtctgttcatgatcataatc |
| | | agccataccacatttgtagaggttttacttgctttaaaaaacctcccacacctccccctgaacctgaaaca |
| | | taaaatgaatgcaattgttgttgttaacttgtttattgcagcttataatggttacaaataaagcaatagcatca |
| | | caaatttcacaaataaagcatttttttcactgcattctagttgtggtttgtccaaactcatcaatgtatcttaac |
| | | gcgtttaccaatgcttaatcagtgaggcacctatctcagcgatctgtctatttcgttcatccatagttgcctg |
| | | actccccgtcgtgtagataactacgatacgggagggcttaccatctggccccagtgctgcaatgatacc |
| | | gcgagacccacgctcaccggctccagatttatcagcaataaaccagccagccggaagggccgagcg |
| | | cagaagtggtcctgcaactttatccgcctccatccagtctattaattgttgccgggaagctagagtaagta |
| | | gttcgccagttaatagtttgcgcaacgttgttgccattgctacaggcatcgtggtgtcacgctcgtcgtttg |
| | | gtatggcttcattcagctccggttcccaacgatcaaggcgagttacatgatcccccatgttgtgcaaaaa |
| | | agcggttagctccttcggtcctccgatcgttgtcagaagtaagttggccgcagtgttatcactcatggtta |
| | | tggcagcactgcataattctcttactgtcatgccatccgtaagatgcttttctgtgactggtgagtactcaa |
| | | ccaagtcattctgagaatagtgtatgcggcgaccgagttgctcttgcccggcgtcaatacgggataata |
| | | ccgcgccacatagcagaactttaaaagtgctcatcattggaaaacgttcttcggggcgaaaactctcaa |
| | | ggatcttaccgctgttgagatccagttcgatgtaacccactcgtgcacccaactgatcttcagcatctttta |
| | | ctttcaccagcgtttctgggtgagcaaaaacaggaaggcaaaatgccgcaaaaaagggaataagggc |
| | | gacacggaaatgttgaatactcatactcttcctttttcaatattattgaagcatttatcagggttattgtctcat |
| | | gagcggatacatatttgaatgtatttagaaaaataaacaaataggggtacgcgtaaattgtaagcgttaat |
| | | attttgttaaaattcgcgttaaatttttgttaaatcagctcattttttaaccaataggccgaaatcggcaaaat |
| | | cccttataaatcaaaagaatagaccgagatagggttgagtgttgttccagtttggaacaagagtccacta |
| | | ttaaagaacgtggactccaacgtcaaagggcgaaaaaccgtctatcagggcgatggcccactacgtg |
| | | aaccatcaccctaatcaagttttttggggtcgaggtgccgtaaagcactaaatcggaaccctaaaggga |
| | | gcccccgatttagagcttgacggggaaagccggcgaacgtggcgagaaaggaagggaagaaagc |
| | | gaaaggagcgggcgctagggcgctggcaagtgtagcggtcacgctgcgcgtaaccaccacacccg |
| | | ccgcgcttaatgcgccgctacagggcgcgtcaggtggcacttttcggggaaatgtgcgcggaacccc |
| | | tatttgtttatttttctaaatacattcaaatatgtatccgctcatgagacaataaccctgataaatgcttcaata |
| | | atattgaaaaaggaagaatcctgaggcggaaagaaccagctgtggaatgtgtgtcagttagggtgtgg |
| | | aaagtccccaggctccccagcaggcagaagtatgcaaagcatgcatctcaattagtcagcaaccagg |
| | | tgtggaaagtccccaggctccccagcaggcagaagtatgcaaagcatgcatctcaattagtcagcaac |
| | | catagtcccgcccctaactccgcccatcccgcccctaactccgcccagttccgcccattctccgcccca |
| | | tggctgactaattttttttatttatgcagaggccgaggccgcctcggcctctgagctattccagaagtagt |
| | | gaggaggcttttttggaggcctaggcttttgcaaatatcgatatggccaagttgaccagtgccgttccgg |
| | | tgctcaccgcgcgcgacgtcgccggagcggtcgagttctggaccgaccggctcgggttctcccggg |
| | | acttcgtggaggacgacttcgccggtgtggtccgggacgacgtgaccctgttcatcagcgcggtcca |
| | | ggaccaggtggtgccggacaacaccctggcctgggtgtgggtgcgcggcctggacgagctgtacg |
| | | ccgagtggtcggaggtcgtgtccacgaacttccgggacgcctccgggccggccatgaccgagatcg |
| | | gcgagcagccgtggggggggagttcgccctgcgcgacccggccggcaactgcgtgcacttcgtg |
| | | gccgaggagcaggactgatgagcgggactctggggttcgaaatgaccgaccaagcgacgcccaac |
| | | ctgccatcacgagatttcgattccaccgccgccttctatgaaaggttgggcttcggaatcgttttccggg |
| | | acgccggctggatgatcctccagcgcggggatctcatgctggagttcttcgcccaccctagggggag |
| | | gctaactgaaacacggaaggagacaataccggaaggaacccgcgctatgacggcaataaaaagac |
| | | agaataaaacgcacggtgttgggtcgtttgttcataaacgcggggttcggtcccagggctggcactctg |
| | | tcgataccccaccgagaccccattggggccaatacgcccgcgtttcttccttttccccaccccaccccc |
| | | caagttcgggtgaaggcccagggctcgcagccaacgtcggggggcaggccctgccatagcctca |
| | | ggttactcatatatactttagattgatttaaaacttcatttttaatttaaaaggatctaggtgaagatcctttttg |
| | | ataatctcatgaccaaaatcccttaacgtgagttttcgttccactgagcgtcagaccccgtagaaaagat |
| | | caaaggatcttcttgagatcctttttttctgcgcgtaatctgctgcttgcaaacaaaaaaaccaccgctacc |
| | | agcggtggtttgtttgccggatcaagagctaccaactctttttccgaaggtaactggcttcagcagagcg |
| | | cagataccaaatactgtccttctagtgtagccgtagttaggccaccacttcaagaactctgtagcaccgc |
| | | ctacatacctcgctctgctaatcctgttaccagtggctgctgccagtggcgataagtcgtgtcttaccgg |
| | | gttggactcaagacgatagttaccggataaggcgcagcggtcgggctgaacggggggttcgtgcac |
| | | acagcccagcttggagcgaacgacctacaccgaactgagatacctacagcgtgagctatgagaaag |
| | | cgccacgcttcccgaagggagaaaggcggacaggtatccggtaagcggcagggtcggaacagga |
| | | gagcgcacgagggagcttccagggggaaacgcctggtatctttatagtcctgtcgggtttcgccacct |
| | | ctgacttgagcgtcgatttttgtgatgctcgtcaggggggcggagcctatggaaaaacgccagcaacg |
| | | cggcctttttacggttcctggccttttgctggccttttgctcacatgttctttcctgcgttatcccctgattctgt |
| | | ggataaccgtattaccgcc |
| 17 | plasmid | atgcattagttattaatagtaatcaattacggggtcattagttcatagcccatatatggagttccgcgttaca |
| | pCP5-B | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaccgg |
| | | tggtaccaggtaagtgtacccaattcgccctatagtgagtcgtattacaattcactcgatcgcccttccca |
| | | acagttgcgcagcctgaatggcgaatggagatccaatttttaagtgtataatgtgttaaactactgattcta |
| | | attgtttgtgtattttagattcacagtcccaaggctcatttcaggcccctcagtcctcacagtctgttcatgat |
| | | cataatcagccataccacatttgtagaggttttacttgctttaaaaaacctcccacacctccccctgaacct |
| | | gaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgt |
| | | atcttaacgcgtttaccaatgcttaatcagtgaggcacctatctcagcgatctgtctatttcgttcatccata |
| | | gttgcctgactccccgtcgtgtagataactacgatacgggagggcttaccatctggccccagtgctgca |
| | | atgataccgcgagacccacgctcaccggctccagatttatcagcaataaaccagccagccggaaggg |
| | | ccgagcgcagaagtggtcctgcaactttatccgcctccatccagtctattaattgttgccgggaagctag |
| | | agtaagtagttcgccagttaatagtttgcgcaacgttgttgccattgctacaggcatcgtggtgtcacgct |
| | | cgtcgtttggtatggcttcattcagctccggttcccaacgatcaaggcgagttacatgatcccccatgttg |
| | | tgcaaaaaagcggttagctccttcggtcctccgatcgttgtcagaagtaagttggccgcagtgttatcac |
| | | tcatggttatggcagcactgcataattctcttactgtcatgccatccgtaagatgcttttctgtgactggtga |
| | | gtactcaaccaagtcattctgagaatagtgtatgcggcgaccgagttgctcttgcccggcgtcaatacg |
| | | ggataataccgcgccacatagcagaactttaaaagtgctcatcattggaaaacgttcttcggggcgaaa |
| | | actctcaaggatcttaccgctgttgagatccagttcgatgtaacccactcgtgcacccaactgatcttcag |
| | | catcttttactttcaccagcgtttctgggtgagcaaaaacaggaaggcaaaatgccgcaaaaaaggga |
| | | ataagggcgacacggaaatgttgaatactcatactcttcctttttcaatattattgaagcatttatcagggtt |
| | | attgtctcatgagcggatacatatttgaatgtatttagaaaaataaacaaataggggtacgcgtaaattgta |
| | | agcgttaatattttgttaaaattcgcgttaaatttttgttaaatcagctcattttttaaccaataggccgaaatc |
| | | ggcaaaatcccttataaatcaaaagaatagaccgagatagggttgagtgttgttccagtttggaacaag |
| | | agtccactattaaagaacgtggactccaacgtcaaagggcgaaaaaccgtctatcagggcgatggcc |
| | | cactacgtgaaccatcaccctaatcaagttttttggggtcgaggtgccgtaaagcactaaatcggaacc |
| | | ctaaagggagcccccgatttagagcttgacggggaaagccggcgaacgtggcgagaaaggaaggg |
| | | aagaaagcgaaaggagcgggcgctagggcgctggcaagtgtagcggtcacgctgcgcgtaaccac |
| | | cacacccgccgcgcttaatgcgccgctacagggcgcgtcaggtggcacttttcggggaaatgtgcgc |
| | | ggaacccctatttgtttatttttctaaatacattcaaatatgtatccgctcatgagacaataaccctgataaat |
| | | gcttcaataatattgaaaaaggaagaatcctgaggcggaaagaaccagctgtggaatgtgtgtcagtta |
| | | gggtgtggaaagtccccaggctccccagcaggcagaagtatgcaaagcatgcatctcaattagtcag |
| | | caaccaggtgtggaaagtccccaggctccccagcaggcagaagtatgcaaagcatgcatctcaatta |
| | | gtcagcaaccatagtcccgcccctaactccgcccatcccgcccctaactccgcccagttccgcccattc |
| | | tccgccccatggctgactaattttttttatttatgcagaggccgaggccgcctcggcctctgagctattcc |
| | | agaagtagtgaggaggcttttttggaggcctaggcttttgcaaatatcgatatggccaagcctttgtctca |
| | | agaagaatccaccctcattgaaagagcaacggctacaatcaacagcatccccatctctgaagactaca |
| | | gcgtcgccagcgcagctctctctagcgacggccgcatcttcactggtgtcaatgtatatcattttactgg |
| | | gggaccttgtgcagaactcgtggtgctgggcactgctgctgctgcggcagctggcaacctgacttgtat |
| | | cgtcgcgatcggaaatgagaacaggggcatcttgagcccctgcggacggtgccgacaggtgcttctc |
| | | gatctgcatcctgggatcaaagccatagtgaaggacagtgatggacagccgacggcagttgggattc |
| | | gtgaattgctgccctctggttatgtgtgggagggctaacttaagagacaggatgaggatcgtttcgcaa |
| | | gagcgggactctggggttcgaaatgaccgaccaagcgacgcccaacctgccatcacgagatttcgat |
| | | tccaccgccgccttctatgaaaggttgggcttcggaatcgttttccgggacgccggctggatgatcctc |
| | | cagcgcggggatctcatgctggagttcttcgcccaccctagggggaggctaactgaaacacggaag |
| | | gagacaataccggaaggaacccgcgctatgacggcaataaaaagacagaataaaacgcacggtgtt |
| | | gggtcgtttgttcataaacgcggggttcggtcccagggctggcactctgtcgataccccaccgagacc |
| | | ccattggggccaatacgcccgcgtttcttccttttccccaccccaccccccaagttcgggtgaaggccc |
| | | agggctcgcagccaacgtcggggcggcaggccctgccatagcctcaggttactcatatatactttaga |
| | | ttgatttaaaacttcatttttaatttaaaaggatctaggtgaagatcctttttgataatctcatgaccaaaatcc |
| | | cttaacgtgagttttcgttccactgagcgtcagaccccgtagaaaagatcaaaggatcttcttgagatcct |
| | | ttttttctgcgcgtaatctgctgcttgcaaacaaaaaaaccaccgctaccagcggtggtttgtttgccgga |
| | | tcaagagctaccaactctttttccgaaggtaactggcttcagcagagcgcagataccaaatactgtcctt |
| | | ctagtgtagccgtagttaggccaccacttcaagaactctgtagcaccgcctacatacctcgctctgctaa |
| | | tcctgttaccagtggctgctgccagtggcgataagtcgtgtcttaccgggttggactcaagacgatagtt |
| | | accggataaggcgcagcggtcgggctgaacggggggttcgtgcacacagcccagcttggagcgaa |
| | | cgacctacaccgaactgagatacctacagcgtgagctatgagaaagcgccacgcttcccgaaggga |
| | | gaaaggcggacaggtatccggtaagcggcagggtcggaacaggagagcgcacgagggagcttcc |
| | | agggggaaacgcctggtatctttatagtcctgtcgggtttcgccacctctgacttgagcgtcgatttttgtg |
| | | atgctcgtcaggggggggagcctatggaaaaacgccagcaacgcggcctttttacggttcctggcct |
| | | tttgctggccttttgctcacatgttctttcctgcgttatcccctgattctgtggataaccgtattaccgcc |
| 18 | plasmid | atgcattagttattaatagtaatcaattacggggtcattagttcatagcccatatatggagttccgcgttaca |
| | pCP5-B1 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggaggcaggtggacaggaaggttc |
| | | taatgttcttaaggcacaggaactgggacatctgggcccggaaagcctttttctctgtgatccggtacag |
| | | tccttctgcggtaccaggtaagtgtacccaattcgccctatagtgagtcgtattacaattcactcgatcgc |
| | | ccttcccaacagttgcgcagcctgaatggcgaatggagatccaatttttaagtgtataatgtgttaaacta |
| | | ctgattctaattgtttgtgtattttagattcacagtcccaaggctcatttcaggcccctcagtcctcacagtct |
| | | gttcatgatcataatcagccataccacatttgtagaggttttacttgctttaaaaaacctcccacacctccc |
| | | cctgaacctgaaacataaaatgaatgcaattgttgttgttaacttgtttattgcagcttataatggttacaaat |
| | | aaagcaatagcatcacaaatttcacaaataaagcatttttttcactgcattctagttgtggtttgtccaaact |
| | | catcaatgtatcttaacgcgtttaccaatgcttaatcagtgaggcacctatctcagcgatctgtctatttcgt |
| | | tcatccatagttgcctgactccccgtcgtgtagataactacgatacgggagggcttaccatctggcccca |
| | | gtgctgcaatgataccgcgagacccacgctcaccggctccagatttatcagcaataaaccagccagcc |
| | | ggaagggccgagcgcagaagtggtcctgcaactttatccgcctccatccagtctattaattgttgccgg |
| | | gaagctagagtaagtagttcgccagttaatagtttgcgcaacgttgttgccattgctacaggcatcgtggt |
| | | gtcacgctcgtcgtttggtatggcttcattcagctccggttcccaacgatcaaggcgagttacatgatccc |
| | | ccatgttgtgcaaaaaagcggttagctccttcggtcctccgatcgttgtcagaagtaagttggccgcagt |
| | | gttatcactcatggttatggcagcactgcataattctcttactgtcatgccatccgtaagatgcttttctgtga |
| | | ctggtgagtactcaaccaagtcattctgagaatagtgtatgcggcgaccgagttgctcttgcccggcgt |
| | | caatacgggataataccgcgccacatagcagaactttaaaagtgctcatcattggaaaacgttcttcgg |
| | | ggcgaaaactctcaaggatcttaccgctgttgagatccagttcgatgtaacccactcgtgcacccaact |
| | | gatcttcagcatcttttactttcaccagcgtttctgggtgagcaaaaacaggaaggcaaaatgccgcaaa |
| | | aaagggaataagggcgacacggaaatgttgaatactcatactcttcctttttcaatattattgaagcatttat |
| | | cagggttattgtctcatgagcggatacatatttgaatgtatttagaaaaataaacaaataggggtacgcgt |
| | | aaattgtaagcgttaatattttgttaaaattcgcgttaaatttttgttaaatcagctcattttttaaccaataggc |
| | | cgaaatcggcaaaatcccttataaatcaaaagaatagaccgagatagggttgagtgttgttccagtttgg |
| | | aacaagagtccactattaaagaacgtggactccaacgtcaaagggcgaaaaaccgtctatcagggcg |
| | | atggcccactacgtgaaccatcaccctaatcaagttttttggggtcgaggtgccgtaaagcactaaatcg |
| | | gaaccctaaagggagcccccgatttagagcttgacggggaaagccggcgaacgtggcgagaaagg |
| | | aagggaagaaagcgaaaggagcgggcgctagggcgctggcaagtgtagcggtcacgctgcgcgt |
| | | aaccaccacacccgccgcgcttaatgcgccgctacagggcgcgtcaggtggcacttttcggggaaat |
| | | gtgcgcggaacccctatttgtttatttttctaaatacattcaaatatgtatccgctcatgagacaataaccct |
| | | gataaatgcttcaataatattgaaaaaggaagaatcctgaggcggaaagaaccagctgtggaatgtgt |
| | | gtcagttagggtgtggaaagtccccaggctccccagcaggcagaagtatgcaaagcatgcatctcaat |
| | | tagtcagcaaccaggtgtggaaagtccccaggctccccagcaggcagaagtatgcaaagcatgcatc |
| | | tcaattagtcagcaaccatagtcccgcccctaactccgcccatcccgcccctaactccgcccagttccg |
| | | cccattctccgccccatggctgactaattttttttatttatgcagaggccgaggccgcctcggcctctgag |
| | | ctattccagaagtagtgaggaggcttttttggaggcctaggcttttgcaaatatcgatatggccaagcctt |
| | | tgtctcaagaagaatccaccctcattgaaagagcaacggctacaatcaacagcatccccatctctgaag |
| | | actacagcgtcgccagcgcagctctctctagcgacggccgcatcttcactggtgtcaatgtatatcatttt |
| | | actgggggaccttgtgcagaactcgtggtgctgggcactgctgctgctgcggcagctggcaacctga |
| | | cttgtatcgtcgcgatcggaaatgagaacaggggcatcttgagcccctgcggacggtgccgacaggt |
| | | gcttctcgatctgcatcctgggatcaaagccatagtgaaggacagtgatggacagccgacggcagttg |
| | | ggattcgtgaattgctgccctctggttatgtgtgggagggctaacttaagagacaggatgaggatcgttt |
| | | cgcaagagcgggactctggggttcgaaatgaccgaccaagcgacgcccaacctgccatcacgagat |
| | | ttcgattccaccgccgccttctatgaaaggttgggcttcggaatcgttttccgggacgccggctggatga |
| | | tcctccagcgcggggatctcatgctggagttcttcgcccaccctagggggaggctaactgaaacacg |
| | | gaaggagacaataccggaaggaacccgcgctatgacggcaataaaaagacagaataaaacgcacg |
| | | gtgttgggtcgtttgttcataaacgcggggttcggtcccagggctggcactctgtcgataccccaccga |
| | | gaccccattggggccaatacgcccgcgtttcttccttttccccaccccaccccccaagttcgggtgaag |
| | | gcccagggctcgcagccaacgtcggggcggcaggccctgccatagcctcaggttactcatatatactt |
| | | tagattgatttaaaacttcatttttaatttaaaaggatctaggtgaagatcctttttgataatctcatgaccaaa |
| | | atcccttaacgtgagttttcgttccactgagcgtcagaccccgtagaaaagatcaaaggatcttcttgag |
| | | atcctttttttctgcgcgtaatctgctgcttgcaaacaaaaaaaccaccgctaccagcggtggtttgtttgc |
| | | cggatcaagagctaccaactctttttccgaaggtaactggcttcagcagagcgcagataccaaatactg |
| | | tccttctagtgtagccgtagttaggccaccacttcaagaactctgtagcaccgcctacatacctcgctctg |
| | | ctaatcctgttaccagtggctgctgccagtggcgataagtcgtgtcttaccgggttggactcaagacgat |
| | | agttaccggataaggcgcagcggtcgggctgaacggggggttcgtgcacacagcccagcttggagc |
| | | gaacgacctacaccgaactgagatacctacagcgtgagctatgagaaagcgccacgcttcccgaag |
| | | ggagaaaggcggacaggtatccggtaagcggcagggtcggaacaggagagcgcacgagggagc |
| | | ttccagggggaaacgcctggtatctttatagtcctgtcgggtttcgccacctctgacttgagcgtcgatttt |
| | | tgtgatgctcgtcaggggggcggagcctatggaaaaacgccagcaacgcggcctttttacggttcctg |
| | | gccttttgctggccttttgctcacatgttctttcctgcgttatcccctgattctgtggataaccgtattaccgc |
| | | c |
| 19 | plasmid | atgcattagttattaatagtaatcaattacggggtcattagttcatagcccatatatggagttccgcgttaca |
| | pCP5-B2 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccagggc |
| | | gaattccagcacactggcggccgttactagtggatccgagctcggtaccaagcttcgaggcaggtgg |
| | | acagcttggttctaatgaagttaaccctgtcgttctgcgacatctgggcccggaaagcgtttaactgatg |
| | | gatggaacagtccttctgcggtaccaggtaagtgtacccaattcgccctatagtgagtcgtattacaattc |
| | | actcgatcgcccttcccaacagttgcgcagcctgaatggcgaatggagatccaatttttaagtgtataat |
| | | gtgttaaactactgattctaattgtttgtgtattttagattcacagtcccaaggctcatttcaggcccctcagt |
| | | cctcacagtctgttcatgatcataatcagccataccacatttgtagaggttttacttgctttaaaaaacctcc |
| | | cacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtt |
| | | tgtccaaactcatcaatgtatcttaacgcgtttaccaatgcttaatcagtgaggcacctatctcagcgatct |
| | | gtctatttcgttcatccatagttgcctgactccccgtcgtgtagataactacgatacgggagggcttacca |
| | | tctggccccagtgctgcaatgataccgcgagacccacgctcaccggctccagatttatcagcaataaa |
| | | ccagccagccggaagggccgagcgcagaagtggtcctgcaactttatccgcctccatccagtctatta |
| | | attgttgccgggaagctagagtaagtagttcgccagttaatagtttgcgcaacgttgttgccattgctaca |
| | | ggcatcgtggtgtcacgctcgtcgtttggtatggcttcattcagctccggttcccaacgatcaaggcgag |
| | | ttacatgatcccccatgttgtgcaaaaaagcggttagctccttcggtcctccgatcgttgtcagaagtaag |
| | | ttggccgcagtgttatcactcatggttatggcagcactgcataattctcttactgtcatgccatccgtaaga |
| | | tgcttttctgtgactggtgagtactcaaccaagtcattctgagaatagtgtatgcggcgaccgagttgctc |
| | | ttgcccggcgtcaatacgggataataccgcgccacatagcagaactttaaaagtgctcatcattggaaa |
| | | acgttcttcggggcgaaaactctcaaggatcttaccgctgttgagatccagttcgatgtaacccactcgt |
| | | gcacccaactgatcttcagcatcttttactttcaccagcgtttctgggtgagcaaaaacaggaaggcaaa |
| | | atgccgcaaaaaagggaataagggcgacacggaaatgttgaatactcatactcttcctttttcaatattat |
| | | tgaagcatttatcagggttattgtctcatgagcggatacatatttgaatgtatttagaaaaataaacaaata |
| | | ggggtacgcgtaaattgtaagcgttaatattttgttaaaattcgcgttaaatttttgttaaatcagctcatttttt |
| | | aaccaataggccgaaatcggcaaaatcccttataaatcaaaagaatagaccgagatagggttgagtgtt |
| | | gttccagtttggaacaagagtccactattaaagaacgtggactccaacgtcaaagggcgaaaaaccgt |
| | | ctatcagggcgatggcccactacgtgaaccatcaccctaatcaagttttttggggtcgaggtgccgtaa |
| | | agcactaaatcggaaccctaaagggagcccccgatttagagcttgacggggaaagccggcgaacgt |
| | | ggcgagaaaggaagggaagaaagcgaaaggagcgggcgctagggcgctggcaagtgtagcggt |
| | | cacgctgcgcgtaaccaccacacccgccgcgcttaatgcgccgctacagggcgcgtcaggtggcac |
| | | ttttcggggaaatgtgcgcggaacccctatttgtttatttttctaaatacattcaaatatgtatccgctcatga |
| | | gacaataaccctgataaatgcttcaataatattgaaaaaggaagaatcctgaggcggaaagaaccagc |
| | | tgtggaatgtgtgtcagttagggtgtggaaagtccccaggctccccagcaggcagaagtatgcaaagc |
| | | atgcatctcaattagtcagcaaccaggtgtggaaagtccccaggctccccagcaggcagaagtatgca |
| | | aagcatgcatctcaattagtcagcaaccatagtcccgcccctaactccgcccatcccgcccctaactcc |
| | | gcccagttccgcccattctccgccccatggctgactaattttttttatttatgcagaggccgaggccgcct |
| | | cggcctctgagctattccagaagtagtgaggaggcttttttggaggcctaggcttttgcaaatatcgatat |
| | | ggccaagcctttgtctcaagaagaatccaccctcattgaaagagcaacggctacaatcaacagcatcc |
| | | ccatctctgaagactacagcgtcgccagcgcagctctctctagcgacggccgcatcttcactggtgtca |
| | | atgtatatcattttactgggggaccttgtgcagaactcgtggtgctgggcactgctgctgctgcggcagc |
| | | tggcaacctgacttgtatcgtcgcgatcggaaatgagaacaggggcatcttgagcccctgcggacggt |
| | | gccgacaggtgcttctcgatctgcatcctgggatcaaagccatagtgaaggacagtgatggacagcc |
| | | gacggcagttgggattcgtgaattgctgccctctggttatgtgtgggagggctaacttaagagacagga |
| | | tgaggatcgtttcgcaagagcgggactctggggttcgaaatgaccgaccaagcgacgcccaacctgc |
| | | catcacgagatttcgattccaccgccgccttctatgaaaggttgggcttcggaatcgttttccgggacgc |
| | | cggctggatgatcctccagcgcggggatctcatgctggagttcttcgcccaccctagggggaggctaa |
| | | ctgaaacacggaaggagacaataccggaaggaacccgcgctatgacggcaataaaaagacagaat |
| | | aaaacgcacggtgttgggtcgtttgttcataaacgcggggttcggtcccagggctggcactctgtcgat |
| | | accccaccgagaccccattggggccaatacgcccgcgtttcttccttttccccaccccaccccccaagt |
| | | tcgggtgaaggcccagggctcgcagccaacgtcggggcggcaggccctgccatagcctcaggttac |
| | | tcatatatactttagattgatttaaaacttcatttttaatttaaaaggatctaggtgaagatcctttttgataatct |
| | | catgaccaaaatcccttaacgtgagttttcgttccactgagcgtcagaccccgtagaaaagatcaaagg |
| | | atcttcttgagatcctttttttctgcgcgtaatctgctgcttgcaaacaaaaaaaccaccgctaccagcggt |
| | | ggtttgtttgccggatcaagagctaccaactctttttccgaaggtaactggcttcagcagagcgcagata |
| | | ccaaatactgtccttctagtgtagccgtagttaggccaccacttcaagaactctgtagcaccgcctacat |
| | | acctcgctctgctaatcctgttaccagtggctgctgccagtggcgataagtcgtgtcttaccgggttgga |
| | | ctcaagacgatagttaccggataaggcgcagcggtcgggctgaacggggggttcgtgcacacagcc |
| | | cagcttggagcgaacgacctacaccgaactgagatacctacagcgtgagctatgagaaagcgccac |
| | | gcttcccgaagggagaaaggcggacaggtatccggtaagcggcagggtcggaacaggagagcgc |
| | | acgagggagcttccagggggaaacgcctggtatctttatagtcctgtcgggtttcgccacctctgacttg |
| | | agcgtcgatttttgtgatgctcgtcaggggggcggagcctatggaaaaacgccagcaacgcggccttt |
| | | ttacggttcctggccttttgctggccttttgctcacatgttctttcctgcgttatcccctgattctgtggataac |
| | | cgtattaccgcc |
| 20 | plasmid | atgcattagttattaatagtaatcaattacggggtcattagttcatagcccatatatggagttccgcgttaca |
| | pCP5-B3 | taacttacggtaaatggcccgcctggctgaccgcccaacgacccccgcccattgacgtcaataatgac |
| | | gtatgttcccatagtaacgccaatagggactttccattgacgtcaatgggtggagtatttacggtaaactg |
| | | cccacttggcagtacatcaagtgtatcatatgccaagtacgccccctattgacgtcaatgacggtaaatg |
| | | gcccgcctggcattatgcccagtacatgaccttatgggactttcctacttggcagtacatctacgtattagt |
| | | catcgctattaccatggtgatgcggttttggcagtacatcaatgggcgtggatagcggtttgactcacgg |
| | | ggatttccaagtctccaccccattgacgtcaatgggagtttgttttggcaccaaaatcaacgggactttcc |
| | | aaaatgtcgtaacaactccgccccattgacgcaaatgggcggtaggcgtgtacggtgggaggtctata |
| | | taagcagagctggtttagtgaaccgtcagatccgctagcgattacgccaagctcgaaattaaccctcac |
| | | taaagggaacaaaagctggagctcgttaattaaaagcttctcgagagtttaaacaggcgcgccaaggg |
| | | cgaattcggatccgcggccgccttaagctcgaggcaggtggacaggaaggttctaatgttctataggg |
| | | tctgcttgtcgctcatctgggcccggagatgcgtaaagtcagacatccggtacagtccttctgcggtacc |
| | | aggtaagtgtacccaattcgccctatagtgagtcgtattacaattcactcgatcgcccttcccaacagttg |
| | | cgcagcctgaatggcgaatggagatccaatttttaagtgtataatgtgttaaactactgattctaattgtttg |
| | | tgtattttagattcacagtcccaaggctcatttcaggcccctcagtcctcacagtctgttcatgatcataatc |
| | | agccataccacatttgtagaggttttacttgctttaaaaaacctcccacacctccccctgaacctgaaaca |
| | | taaaatgaatgcaattgttgttgttaacttgtttattgcagcttataatggttacaaataaagcaatagcatca |
| | | caaatttcacaaataaagcatttttttcactgcattctagttgtggtttgtccaaactcatcaatgtatcttaac |
| | | gcgtttaccaatgcttaatcagtgaggcacctatctcagcgatctgtctatttcgttcatccatagttgcctg |
| | | actccccgtcgtgtagataactacgatacgggagggcttaccatctggccccagtgctgcaatgatacc |
| | | gcgagacccacgctcaccggctccagatttatcagcaataaaccagccagccggaagggccgagcg |
| | | cagaagtggtcctgcaactttatccgcctccatccagtctattaattgttgccgggaagctagagtaagta |
| | | gttcgccagttaatagtttgcgcaacgttgttgccattgctacaggcatcgtggtgtcacgctcgtcgtttg |
| | | gtatggcttcattcagctccggttcccaacgatcaaggcgagttacatgatcccccatgttgtgcaaaaa |
| | | agcggttagctccttcggtcctccgatcgttgtcagaagtaagttggccgcagtgttatcactcatggtta |
| | | tggcagcactgcataattctcttactgtcatgccatccgtaagatgcttttctgtgactggtgagtactcaa |
| | | ccaagtcattctgagaatagtgtatgcggcgaccgagttgctcttgcccggcgtcaatacgggataata |
| | | ccgcgccacatagcagaactttaaaagtgctcatcattggaaaacgttcttcggggcgaaaactctcaa |
| | | ggatcttaccgctgttgagatccagttcgatgtaacccactcgtgcacccaactgatcttcagcatctttta |
| | | ctttcaccagcgtttctgggtgagcaaaaacaggaaggcaaaatgccgcaaaaaagggaataagggc |
| | | gacacggaaatgttgaatactcatactcttcctttttcaatattattgaagcatttatcagggttattgtctcat |
| | | gagcggatacatatttgaatgtatttagaaaaataaacaaataggggtacgcgtaaattgtaagcgttaat |
| | | attttgttaaaattcgcgttaaatttttgttaaatcagctcattttttaaccaataggccgaaatcggcaaaat |
| | | cccttataaatcaaaagaatagaccgagatagggttgagtgttgttccagtttggaacaagagtccacta |
| | | ttaaagaacgtggactccaacgtcaaagggcgaaaaaccgtctatcagggcgatggcccactacgtg |
| | | aaccatcaccctaatcaagttttttggggtcgaggtgccgtaaagcactaaatcggaaccctaaaggga |
| | | gcccccgatttagagcttgacggggaaagccggcgaacgtggcgagaaaggaagggaagaaagc |
| | | gaaaggagcgggcgctagggcgctggcaagtgtagcggtcacgctgcgcgtaaccaccacacccg |
| | | ccgcgcttaatgcgccgctacagggcgcgtcaggtggcacttttcggggaaatgtgcgcggaacccc |
| | | tatttgtttatttttctaaatacattcaaatatgtatccgctcatgagacaataaccctgataaatgcttcaata |
| | | atattgaaaaaggaagaatcctgaggcggaaagaaccagctgtggaatgtgtgtcagttagggtgtgg |
| | | aaagtccccaggctccccagcaggcagaagtatgcaaagcatgcatctcaattagtcagcaaccagg |
| | | tgtggaaagtccccaggctccccagcaggcagaagtatgcaaagcatgcatctcaattagtcagcaac |
| | | catagtcccgcccctaactccgcccatcccgcccctaactccgcccagttccgcccattctccgcccca |
| | | tggctgactaattttttttatttatgcagaggccgaggccgcctcggcctctgagctattccagaagtagt |
| | | gaggaggcttttttggaggcctaggcttttgcaaatatcgatatggccaagcctttgtctcaagaagaatc |
| | | caccctcattgaaagagcaacggctacaatcaacagcatccccatctctgaagactacagcgtcgcca |
| | | gcgcagctctctctagcgacggccgcatcttcactggtgtcaatgtatatcattttactgggggaccttgt |
| | | gcagaactcgtggtgctgggcactgctgctgctgcggcagctggcaacctgacttgtatcgtcgcgat |
| | | cggaaatgagaacaggggcatcttgagcccctgcggacggtgccgacaggtgcttctcgatctgcat |
| | | cctgggatcaaagccatagtgaaggacagtgatggacagccgacggcagttgggattcgtgaattgct |
| | | gccctctggttatgtgtgggagggctaacttaagagacaggatgaggatcgtttcgcaagagcgggac |
| | | tctggggttcgaaatgaccgaccaagcgacgcccaacctgccatcacgagatttcgattccaccgccg |
| | | ccttctatgaaaggttgggcttcggaatcgttttccgggacgccggctggatgatcctccagcgcgggg |
| | | atctcatgctggagttcttcgcccaccctagggggaggctaactgaaacacggaaggagacaatacc |
| | | ggaaggaacccgcgctatgacggcaataaaaagacagaataaaacgcacggtgttgggtcgtttgttc |
| | | ataaacgcggggttcggtcccagggctggcactctgtcgataccccaccgagaccccattggggcca |
| | | atacgcccgcgtttcttccttttccccaccccaccccccaagttcgggtgaaggcccagggctcgcagc |
| | | caacgtcggggcggcaggccctgccatagcctcaggttactcatatatactttagattgatttaaaacttc |
| | | atttttaatttaaaaggatctaggtgaagatcctttttgataatctcatgaccaaaatcccttaacgtgagtttt |
| | | cgttccactgagcgtcagaccccgtagaaaagatcaaaggatcttcttgagatcctttttttctgcgcgta |
| | | atctgctgcttgcaaacaaaaaaaccaccgctaccagcggtggtttgtttgccggatcaagagctacca |
| | | actctttttccgaaggtaactggcttcagcagagcgcagataccaaatactgtccttctagtgtagccgta |
| | | gttaggccaccacttcaagaactctgtagcaccgcctacatacctcgctctgctaatcctgttaccagtg |
| | | gctgctgccagtggcgataagtcgtgtcttaccgggttggactcaagacgatagttaccggataaggc |
| | | gcagcggtcgggctgaacggggggttcgtgcacacagcccagcttggagcgaacgacctacaccg |
| | | aactgagatacctacagcgtgagctatgagaaagcgccacgcttcccgaagggagaaaggcggaca |
| | | ggtatccggtaagcggcagggtcggaacaggagagcgcacgagggagcttccagggggaaacgc |
| | | ctggtatctttatagtcctgtcgggtttcgccacctctgacttgagcgtcgatttttgtgatgctcgtcaggg |
| | | gggcggagcctatggaaaaacgccagcaacgcggcctttttacggttcctggccttttgctggccttttg |
| | | ctcacatgttctttcctgcgttatcccctgattctgtggataaccgtattaccgcc |
| 21 | signaling | gccagtcccagttcctgtgccttaagaacctcgc |
| | probe to | |
| | detection | |
| | tag A | |
| 22 | signaling | gcgagtcgcagaacgacagggttaacttcctcgc |
| | probe to | |
| | detection | |
| | tag B | |
| 23 | signaling | gcgagagcgacaagcagaccctatagaacctcgc |
| | probe to | |
| | detection | |
| | tag C | |
| 24 | tas 1r2-a | gcgaggagacggatgaggtgaaatagaagctcgc |
| 25 | tas1r2-b | gcgagattgtgaagcagaggggaaagaggctcgc |
| 26 | tas1r2-c | gcgagaccttcacttcatactccttgcacctcgc |
| 27 | tas1r3-a | gcgaggtttcccctgatttcctgtgttccgtcgc |
| 28 | tas1r3-b | gcgagcacccacagcttcaggtcgtactcctcgc |
| 29 | tas1r3-c | gcgaggaccactcatcctggccacaaaagctcgc |
| 30 | Detection | gcaggtggacaggaaggttctaatgttcttaaggcacaggaactgggacatctgggcccggaaagcc |
| | tag A | tttttctctgtgatccggtacagtccttctgc |
| 31 | Detection | gcaggtggacagcttggttctaatgaagttaaccctgtcgttctgcgacatctgggcccggaaagcgttt |
| | tag B | aactgatggatggaacagtccttctgc |
| 32 | Detection | gcaggtggacaggaaggttctaatgttctatagggtctgcttgtcgctcatctgggcccggagatgcgt |
| | tag C | aaagtcagacatccggtacagtccttctgc |
| 33 | Spacer 1 | agggcgaattccagcacactggcggccgttactagtggatccgagctcggag |
| 34 | Spacer 2 | agggcgaattccagcacactggcggccgttactagtggatccgagctcggtaccaagcttcgag |
| 35 | Spacer 3 | aagggcgaattcggatccgcggccgccttaagctcgag |
| 36 | RNA | gcagguggacaggaagguucuaauguucuuaaggcacaggaacugggacaucugggcccgg |
| | sequence | aaagccuuuuucucugugauccgguacaguccuucugc |
| | corresponding | |
| | to | |
| | detection | |
| | tag A | |
| 37 | RNA | gcagguggacagcuugguucuaaugaaguuaacccugucguucugcgacaucugggcccgg |
| | sequence | aaagcguuuaacugauggauggaacaguccuucugc |
| | corresponding | |
| | to | |
| | detection | |
| | tag B | |
| 38 | RNA | gcagguggacaggaagguucuaauguucuauagggucugcuugucgcucaucugggcccg |
| | sequence | gagaugcguaaagucagacauccgguacaguccuucugc |
| | corresponding | |
| | to | |
| | detection | |
| | tag C | |

### EXAMPLES

In order that this disclosure be more fully understood, the following examples are set forth. These examples are only for the purpose of illustration and are not to be construed as limiting the scope of the disclosure in any way.

### Example 1

Production of cell lines: Each gene of interest was subcloned into an independent mammalian expression vector containing a detection tag and drug selectable marker and transfected into HEK293T and CHO cells cultured using DMEM supplemented with 50 mL fetal bovine serum, a 4 mM L-Glutamine, 10 mM Hepes pH 7.4. After 1 to 3 days cells were transferred to fresh media containing mild selective pressure for 7 to 14 days. On the day of the experiment, each well of four to six 24-well tissue culture plates was plated with 200 ul to 400 ul of transfected cells at 1.25 to 2×10⁶ cells/mL in serum-free media and exposed to differentially-labeled signaling probes that can hybridize with the expression products. For each well, 1.7 to 2.5 uL of each probe at a concentration of 2.5 to 5 uM was added to 50 uL of serum free media in tube A and 0.3 to 0.75 uL of transfection reagent, using Lipofectamine for HEK cells (Thermo Fisher 18324) and jetPEI for CHO (Polyplus Transfection 101) was added to tube B. The contents of tubes A and B were mixed and incubated at room temperature for 25 to 30 minutes prior to addition to the well. Following a 1 to 2 hour incubation at 37° C. in a tissue culture incubator, serum-free media was added to the well up to a total volume of 1 mL. The plates were spun at 2,000 RPM for 4 minutes. 750 uL of media was aspirated and replaced with 250 uL of fresh serum-free media. The cells were incubated for an additional 2 to 2.5 hours then used to isolate individual positive cells using Beckman Coulter Epic Altra or Becton Dickinson FACS Aria flow cytometers.

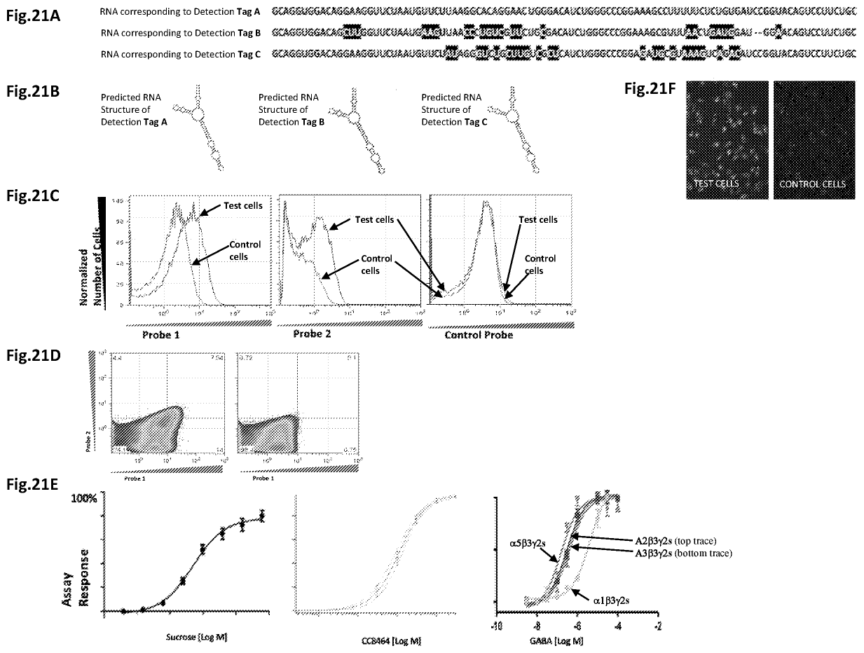

Gene-specific or fluorogenic probes directed to plasmid-encoded sequences downstream of the 3′ stop codon of the cDNAs of interest were used. A first detection tag comprising portions of the reverse-complement of the mRNA sequence of the human Vav gene and two derivate detection tag sequences were generated and used. Gene-specific signaling probes and signaling probes directed to the detection tags including fluorophores and quenchers are shown below. BHQ2 was also used. 5-Methyl-dC and 2-Amino dA chemical modifications were included for the underlined bases (see, FIG. 21) except in probes used for the ENaC cell line. Serum-free media used included DMEM and Opti-MEM Serum Reduced Media.

Signaling probes to detection tags a, b and c:

A:
5′-Cy5 GCCAGTCCCAGTTCCTGTGCCTTAAGAACCTCGC BHQ3-3′
B:
5′-CY5.5 GCGAGTCGCAGAACGACAGGGTTAACTTCCTCGC BHQ3-3′
C
5′-Fam GCGAGAGCGACAAGCAGACCCTATAGAACCTCGC BHQ1-3′

From top to bottom, the sequences of above correspond to SEQ IDs 21, 22 and 23.
GENE-SPECIFIC signaling probes TO tas1/r2 tas1/r3:

tas1r2
5′-CY5.5 GCGAGGAGACGGATGAGGTGAAATAGAAGCTCGC BHQ2-3′
5′-CY5.5 GCGAGATTGTGAAGCAGAGGGGAAAGAGGCTCGC BHQ2-3′
5′-CY5.5 GCGAGACCTTCACTTCATACTCCTTGCACCTCGC BHQ2-3′

From top to bottom, the DNA sequences of above correspond to SEQ IDs 24, 25 and 26

tas1r3
5′-6-FAM GCGAGGTTTCCCCTGATTTCCTGTGTTCCGTCGC BHQ1-3′
5′-6-FAM GCGAGCACCCACAGCTTCAGGTCGTACTCCTCGCBHQ1-3′
5′-6-FAM GCGAGGACCACTCATCCTGGCCACAAAAGCTCGC BHQ1-3′

From top to bottom, the DNA sequences of above correspond to SEQ IDs 27, 28 and 29

Individual positive cells and expanded in 96-well multitier plates. Cell lines were binned based on growth rate, maintained using automated cell culture methods and functional cell-based assays were to select final clones. The ENaC cell line was created without robotic cell culture methods and using a custom low-sodium F-12 HAM media (Sigma N4888) formulation was used for cell culture (Shekdar K and Langer J (2017) Cell lines expressing ENaC and methods of using them, U.S. Pat. No. 9,534,035).

### Example 2

Cell-based Assays: Functional testing using fluorescent cell-based assays in the absence of any antibiotic-mediated selectable pressure was performed over time (typically for at least 4 to 6 weeks) to identify inherently stable clones. Commonly available fluorescent membrane potential and calcium flux kits and reagents were used for fluorescent cell-based assays in 96-well and 384-well plates using the Molecular Devices FLIPR 3 and Hamamatsu FDSS 6000 plate readers as described (Shekdar 2011; Shekdar and Sawchuk 2012; Shekdar and Venkatachalan 2012, Shekdar 2017). Electrophysiology of ion channel cell lines was obtained using the Nanion Technologies Patchliner patch clamp system. The patch clamp recordings were conducted according to Nanion's standard procedure (Obergrussberger 2014). Currents were measured using the whole-cell configuration. The extracellular solution contained the following: 140 mM NaCl, 4 mM KCl, 1 mM MgCl₂, 2 mM CaCl₂), 5 mM glucose, 10 mM Hepes, pH 7.4 (adjusted with NaOH). The intracellular solution for sodium current recordings contained the following: 50 mM CsCl, 10 mM NaCl, 60 mM CsF, 20 mM EGTA, 10 mM Hepes, pH 7.2 (adjusted with CsOH). For transient transfection studies, Fugene 6 was used to transfect expression plasmid into cells cultured as above and cells were assayed three days post-transfection.

### Example 3

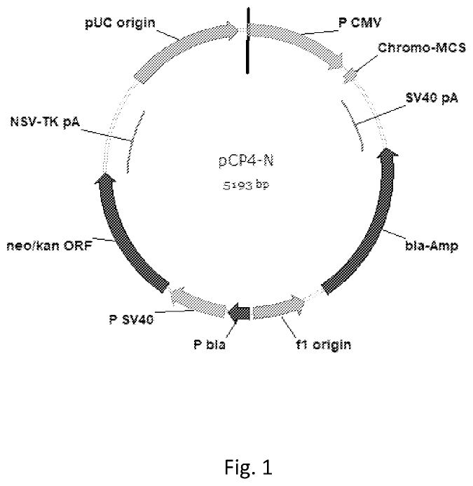

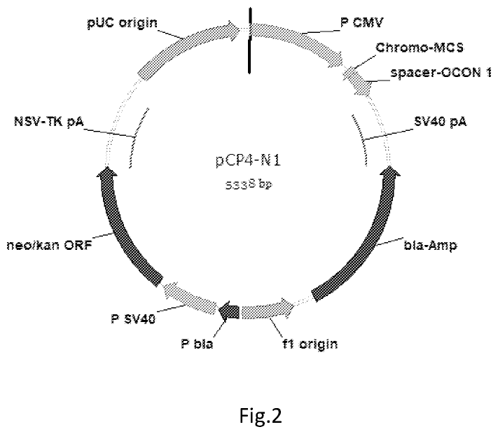

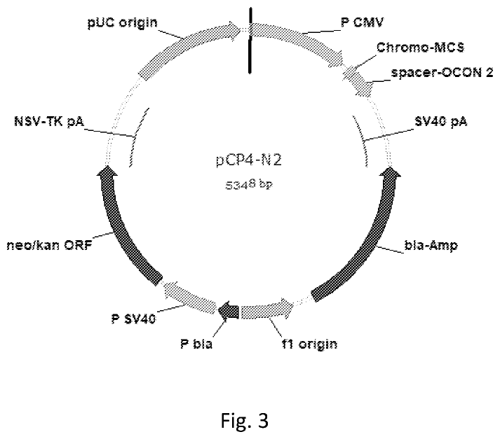

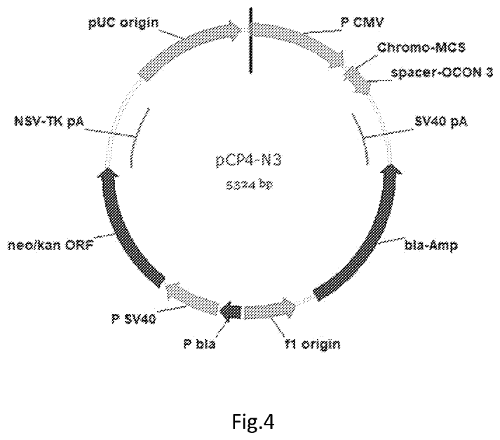

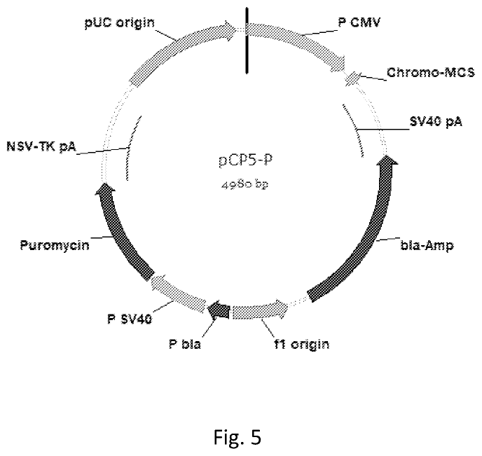

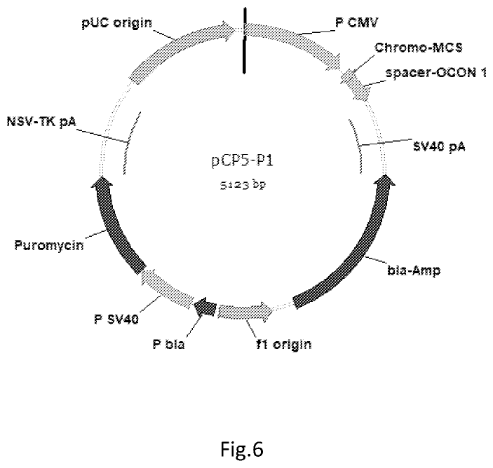

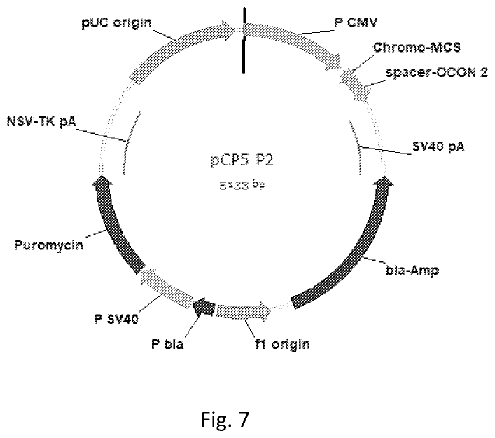

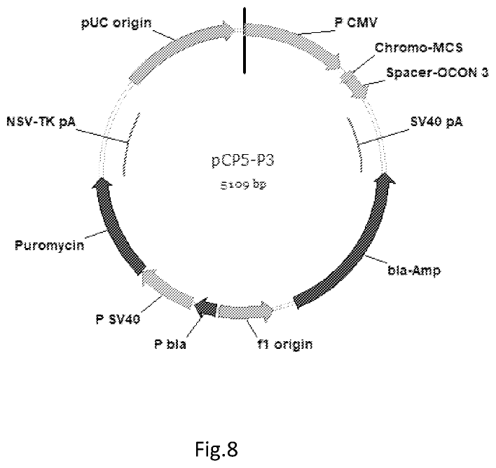

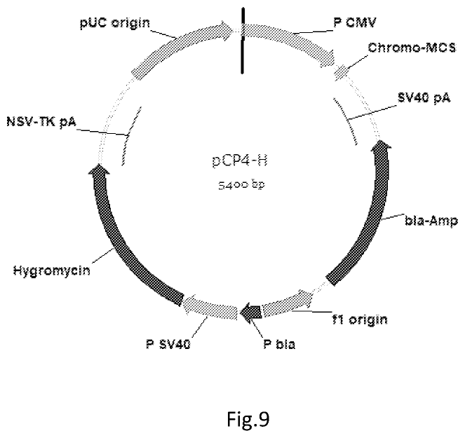

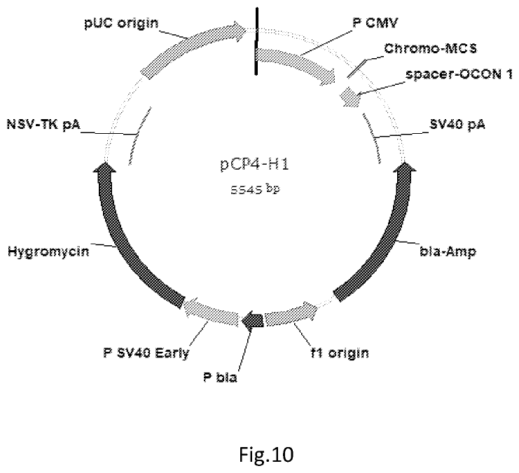

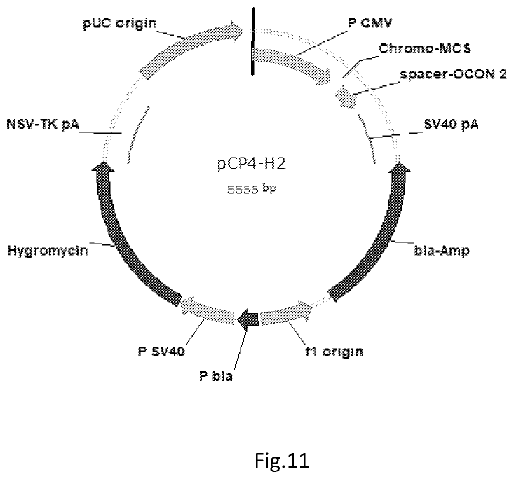

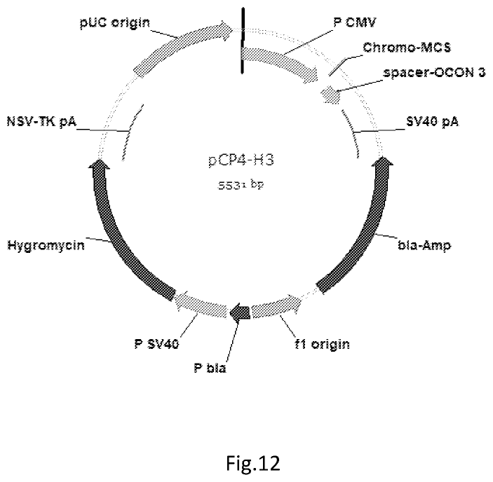

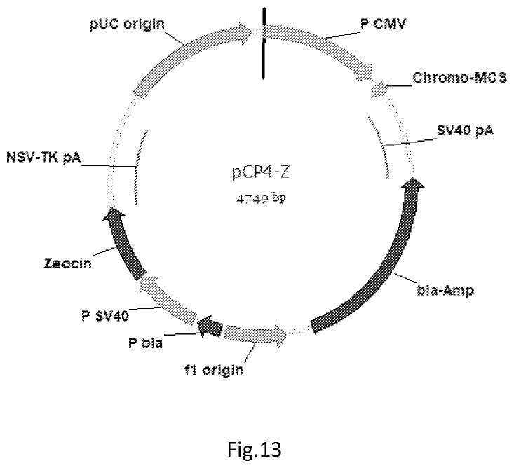

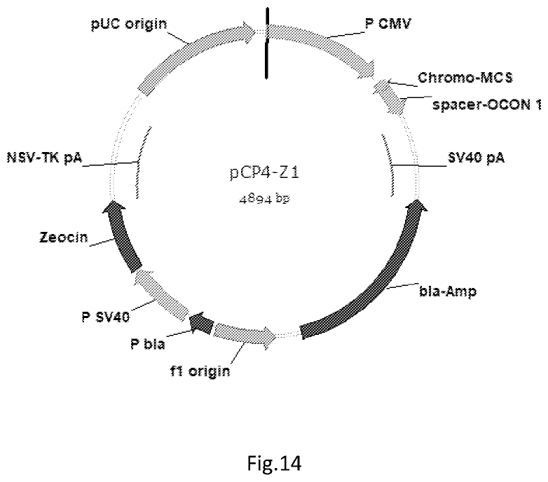

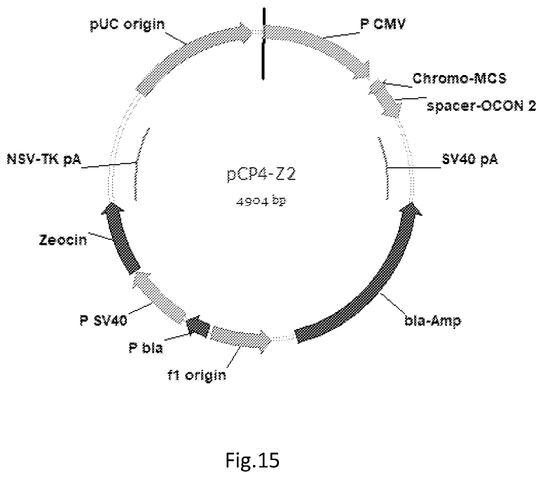

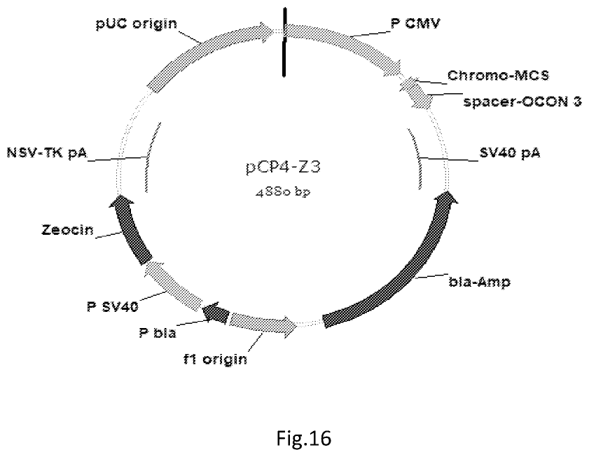

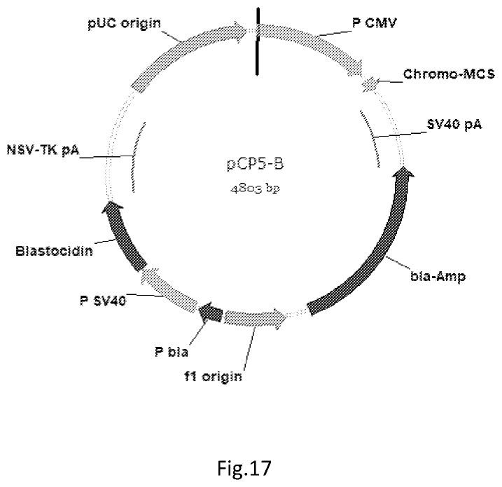

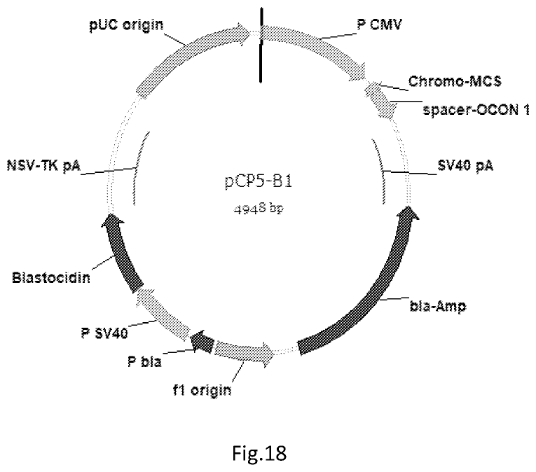

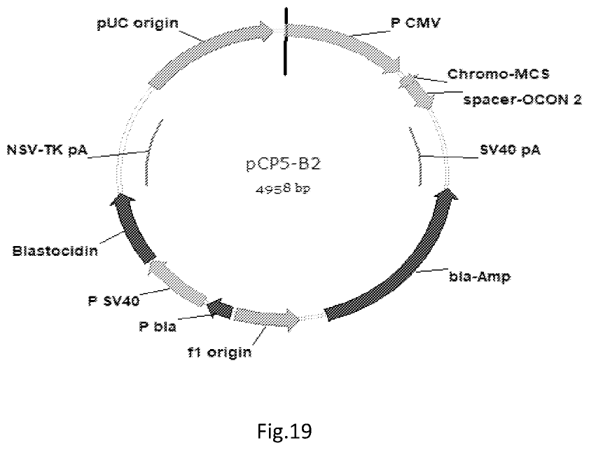

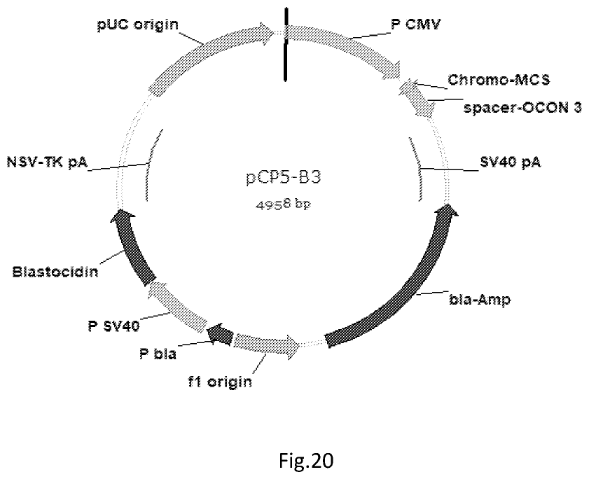

Results obtained from cell lines prepared and assayed as described in Examples 1 and 2 are presented for cell lines prepared using gene-specific signaling probes as well as probes directed to three plasmid-encoded detection tag as disclosed herein. Three detection tags that vary by at least 50% in pairwise comparisons over the 24-nucleotide region indicated and that share similar predicted mRNA folding based on the mFold algorithm (Zuker M (2003) Mfold web server for nucleic acid folding and hybridization prediction, Nucleic Acids Res 31:3406-15) were designed, as shown in FIG. 21A and FIG. 21B, respectively. Representative results for the multiplexed detection of the three detection tag sequences expressed in cells co-transfected with expression plasmids each comprising an expression cassette encoding one of the detection tags using differentially-labeled signaling probes directed to the 24-nucleotide region of the detection tags are shown in FIGS. 21C-E. The functional cell-based response of the heterodimeric human T1R2/T1R3 GPCR sweet taste receptor cell line made using gene-specific probes is shown in FIG. 21F, left panel. This cell line was created by exposing mammalian cell cultures to tas1r2 and tas2r3 gene-specific probes with no prior introduction of any cDNAs. All other cell lines, for example the NaV1.7-αρ1β2 cell line, were created using signaling probes directed to detection tagged expression products of transfected cDNAs of interest. The functional cell-based response of the NaV1.7 cell line to a NaV1.7 inhibitor (U.S. Pat. No. 9,458,118 issued Oct. 4, 2016), is shown in FIG. 1F, center panel.

A combinatorial panel of cell lines and cell-based assays for four GABA_{A} receptor ion channel subunit combinations α1β3γ2s, α2β3γ2s, α3β3γ2s or α5β3γ2s was also generated, as shown in FIG. 21F, right panel. High-throughput screening (HTS) of a library of pharmaceutically active compounds using LOPAC (Sigma 1280) was performed using the panel. 34 LOPAC compounds that activated at least one of the cell lines at least as much as GABA itself were identified, including all known GABA_{A} agonists included in LOPAC: GABA, Propofol, Isoguvacine hydrochloride, Muscimol hydrobromide, Piperidine-4-sulphonic acid, 3-alpha, 21-dihydroxy-5-alpha-pregnan-20-one, 5-alpha-pregnan-3alpha-ol-11,20-dione, 5-alpha-pegnan-3alpha-ol-20-one and Tracazolate. In addition, at least four compounds not previously described as GABA_{A} agonists but known to have other activities associated with GABA_{A} ion channels were identified, including: Etazolate, Androsterone, Chlormezanone and Ivermectin. The screen further identified compounds which, until now, were not reported to interact with GABA_{A}, including: Dipyridamole, Niclosamide, Tyrphostin A9 and I-OMe-Tyrphostin AG 538.

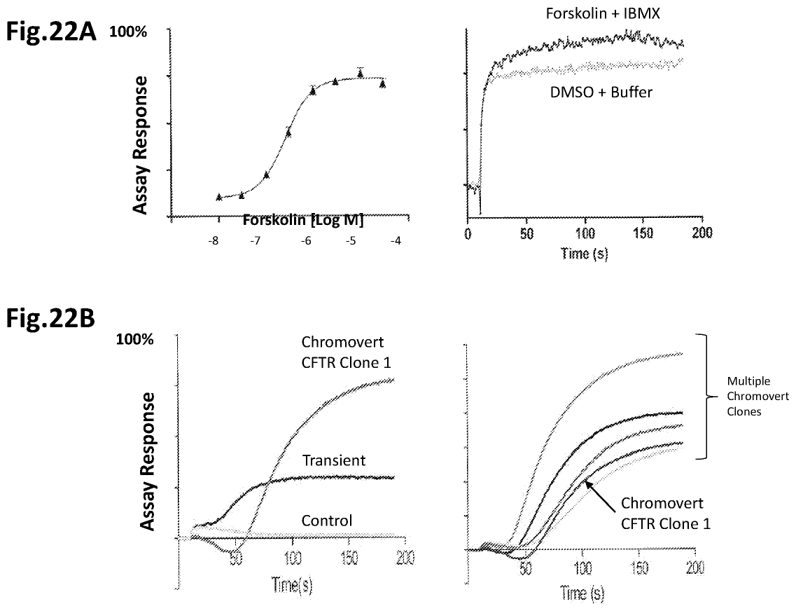

Chromovert® Technology was also used to produce CFTR and CFTR-Δ508 cell lines and cell-based assays. FIG. 22A, left panel shows a dose-response curve for the CFTR cell line to Forskolin. FIG. 22A, right panel shows that the Chromovert® Technology-enabled CFTR-Δ508 cell line enables a functional cell-based assay for CFTR-Δ508 in the absence of any added trafficking correctors. FIG. 22B shows the functional cell-based responses of multiple Chromovert® Technology-enabled CFTR cell lines compared to transiently-transfected cells.

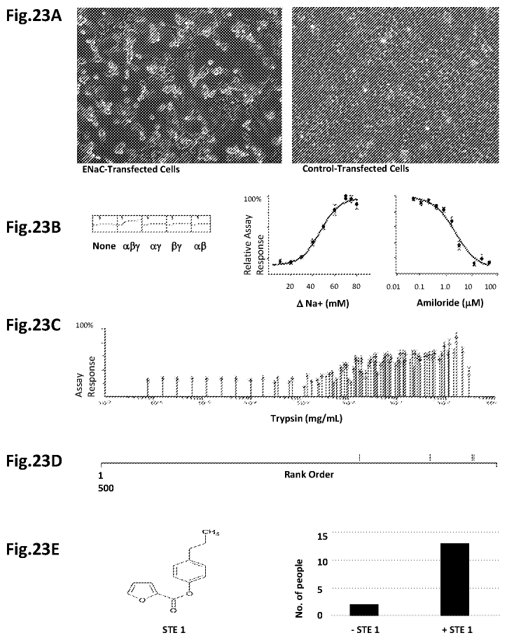

Finally, Chromovert® Technology was used to create a cell line for ENaC. ENaC is cytotoxic when expressed in transfected cell cultures, as shown in FIG. 23A. Chromovert® Technology enabled an αβγ-ENaC cell line as shown in FIG. 23B using a cell-based assay selective for αβγ-ENaC activity. Limited proteolysis of the cell line was used to achieve functionally distinct proteolyzed variants, as shown in FIG. 23C. HTS of a compound library against non-proteolyzed vs. proteolyzed ENaC variants resulted in largely non-overlapping hits, as shown in FIG. 23D. One potentiator identified during HTS of proteolyzed ENaC was assessed in human sensory studies where it was confirmed to enhance the salty taste of sodium chloride, as shown in FIG. 23E.

1. An expression vector comprising a sequence of interest, the vector comprising:
in the following operative order, a promoter, a multiple cloning site, a spacer, and a detection tag.

2. The expression vector of claim 1, wherein the detection tag is an untranslated sequence that is transcribed.

3. The expression vector of claim 1, wherein the spacer sequence comprises from about 4 to about 630 nucleotides.

4. The expression vector of any one of claims 1-3, wherein the spacer sequence comprises the nucleic acid sequence of SEQ ID NO: 33, 34, or 35.

5. The expression vector of any one of claims 1-6, wherein the detection tag comprises the nucleotide sequence set forth in SEQ ID NOs: 30, 31, or 32.

6. The expression vector of any one of claims 1-5 wherein the promoter is selected from the group consisting of a CMV promoter, a TK promoter, a SV40 promoter and an EF-la promoter.

7. The expression vector of claim 6 wherein the promoter is a CMV promoter.

8. The expression vector of any one of claims 1-7, further comprising a first antibiotic selection marker and a second antibiotic selection marker.

9. The expression vector of claim 8, wherein the first antibiotic selection marker is a bacterial selection marker.

10. The expression vector of claim 8 or 9, wherein the second antibiotic selection marker is a mammalian selection marker.

11. The expression vector of any one of claims 1-10, wherein the expression vector comprises the nucleotide sequence set forth in SEQ ID NO: 2, 3, 4, 6, 7, 8, 10, 11, 12, 14, 15, 16, 18, 19, or 20.

12. The expression vector of any one of claims 1-11 further comprising a sequence of interest.

13. The expression vector of claim 12, wherein the sequence of interest is located between the promoter and the spacer.

14. The expression vector of claim 12 or 13, wherein the sequence of interest comprises a gene, a cDNA, or an RNA.

15. The expression vector of claim 14, wherein the sequence of interest comprises a mutated, spliced, or processed form of a gene, cDNA, or an RNA.

16. The expression vector of any one of claims 12-15, wherein the sequence of interest is selected from the group consisting of a GPCR, ion channel and ion channel subunit.

17. The expression vector of claim 16, wherein the ion channel and ion channel subunit is selected from GABAA, CFTR, ENAC and NaV

18. The expression vector of any one of claims 12-15, wherein the sequence interest comprises a human sensory gene, a taste gene, or an odorant receptor gene.

19. The expression vector of claim 18, wherein the human sensory gene, taste gene, or odorant receptor gene is selected form the group consisting of bitter, salt, sour, umami, hot, pepper, cool, mint, fat, fatty acid, kokumi, mouth feel, touch, touch sensation, mechano sensation, pressure sensing and tingle receptor genes.

20. The expression vector of any one of claims 12-15, wherein the sequence of interest comprises a gene from a DNA virus, a gene from an RNA virus, a gene from a retrovirus, a gene from an adeno-associated virus, a gene from an adenovirus, a gene from a lentivirus, a gene from a herpes virus, an HIV gene, or a gene from a coronavirus.

21. The expression vector of any one of claims 12-15, wherein the sequence of interest comprises a gene from SARS, a gene from MERS, or a gene from SARS-CoV-2 virus.

22. The expression vector of any one of claims 12-15, wherein the sequence of interest is selected from a viral gene used to produce viral particles for use in gene or cell therapy.

23. The expression vector of any one of claims 12-15, wherein the sequence of interest is selected from the group consisting of a gene encoding a cellular protein, a gene encoding a membrane protein, a gene encoding a cytosolic protein, a gene encoding a nuclear protein, a gene encoding a multi-subunit protein, and a gene encoding a secreted protein.

24. The expression vector of any one of claims 12-14, wherein the sequence of interest comprises a drug target or a gene encoding a biologic.

25. The expression vector of any one of claims 12-15, wherein the sequence of interest is selected from a gene, an mRNA, or a cDNA involved in unfolded protein response, cell viability, protein production, protein folding, protein assembly, protein modification, glycosylation, proteolysis, secretion, cell membrane integration, or cell surface presentation.

26. The expression vector of any one of claims 12-15, wherein the sequence of interest is selected from the group consisting of full length and spliced ATF, ATF6, ATF6-Alpha, ATF6A, ATF6B, IRE1 alpha, IRE1α, IRE1 beta, IRE1β, PERCDC, ATF4, YY1, NF-YA, NF-YB, NF-YC, XBP1, XBP2, EDEM1, EDEM2, NRF2, HERP, XIAP, GADD34, PPIA, PPIB, PPIG, DNAJC3, DNAJC6, PRDM1, BLIMP-1, CRT, CALR, CNX, PDIA3, ERp57, HSPA5, BiP, SIL1, BAP, DNAJB11, ERdj3, CaBP1, HSP90B1, GRP-94, PDIA4, ERp72, cyclophilin B, SDF2, SDF2L1, ERO1A, ERO1B, ERAD pathway gene, MAN1B1, mannosidase 1, SYN1, HRD1, STC1, STC2, SERCA1, SERCA2, RPGR, COD1, ISYNA1, INO1, SREBP1, SREBP2, SREBP1DC, SREBP2DC, PC, PYC, Sec61A1, Sec61A2, Sec61B, Sec61G, BCL2, BCL2L1, Bcl-2sp, Bcl-XL, BCL2L11, Bim, XRCC6, Ku70, VDAC2, BCAP31, BAP31, YWHAE, and 14-3-3.

27. A method for isolating a cell expressing a gene of interest comprising
(a) providing a mixed population of cells at a concentration from about 0.125 to about 20×10⁶ cells/mL in a medium wherein at least a portion of the mixed population of cells expresses the gene of interest;
(b) exposing the mixed population of cells to about 0.01625 nM to 27.778 uM of a fluorescent signaling probe;
(c) detecting fluoresce of cells within the mixed population of cells; and
(d) isolating a cell that fluoresces above background, wherein fluorescence above background indicates that the cell expresses the gene of interest.

28. The method of claim 27 further wherein the mixed population of cells is exposed to the fluorescent signaling probe is provided at a concentration from about 0.25 uM to about 50 uM in a second medium in step (b).

29. The method of claim 27 or claim 28, wherein the signaling probe hybridizes with to the gene of interest.

30. The method of any one of claims 27-29, wherein the fluorescent signaling probe further comprises a quencher.

31. The method of any one of claims 27-30, wherein the cell expresses a second gene of interest.

32. The method of claim 31 wherein step (b) further comprises exposing the mixed population of cells to a second fluorescent signaling probe.

33. The method of claim 32, wherein the first and second fluorescent signaling probes comprise different fluorophores.

34. The method of any one of claims 27-33 wherein step (b) is performed using a transfection reagent.

35. The method of claim 34, wherein the transfection reagent is Lipofectamine or jetPEI.

36. The method of claim 34 or 35, wherein the transfection agent is provided at a concentration from about 1.875 μM to 13.636 uM.

37. The method of any one of claims 37-33, wherein step (b) is performed using electroporation.

38. The method of any one of claims 27-37, wherein the medium in step (a) is a serum-free medium.

39. The method of any one of claims 28-38 wherein the second medium in step (b) is a serum-free medium.

40. The method of claim 39, wherein the media in step (a) and step (b) are selected from the group consisting of Dulbecco's Modified Eagle Medium (DMEM), Opi-MEM Reduced Serum Media and Ham's F12 Nutrient Mixture media.

41. The method of any one of claims 27-40, wherein the fluorescent singling probe comprises the nucleotide sequence as set forth in SEQ ID NO: 21, 22 or 23.

42. The method of any one of claims 27-41, wherein the gene of interest comprises a detection tag comprising the nucleotide sequence set forth in SEQ ID NO: 30, 31, or 32.

43. The method of any one of claims 27-42, wherein isolating a cell that fluoresces above background, is performed using flow cytometry.

44. The method of any one of claims 27-43 wherein the cell is a eukaryotic or prokaryotic cell.

45. The method any one of claims 27-43, wherein the cell is a mammalian cell.

46. The method of claim 45, wherein the mammalian cell is selected from NS0, CHO, COS, Perc6, HEK-293, HEK 293T, HUVECs, 3T3 and HeLa cells.

47. The method of claim 45 wherein the mammalian cell is selected from CHO, HEK-293 and HEK 293T cells.

48. The method of any one of claims 27-43, wherein the cell is a yeast, insect, fungus, or plant, human, primate, bovine, porcine, feline, marsupial, or murine cell.

49. The method of any one of claims 27-48 wherein the cell is selected from primary, transformed, oncogenically transformed, virally transformed, immortalized, conditionally transformed, explants, or a cell from a tissue section.

50. The method of any one of claims 27-43 wherein, the cell is selected from the group consisting of Chinese hamster ovary (CHO) cells, human embryonic kidney (HEK) cells, cells of a cell line, established neuronal cell lines, pheochromocytomas, neuroblastomas fibroblasts, rhabdomyosarcomas, dorsal root ganglion cells, NSO cells, CV-1 (ATCC CCL 70), COS-1 (ATCC CRL 1650), COS-7 (ATCC CRL 1651), CHO/dhFr- (ATCC CCR-9096), CHO 1-15 (ATCC CRL 9609), 16.4 CHO (ATCC CRL-12023), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), L-cells, HEK-293 (ATCC CRL1573), HEK-293 (ECACC 85120602), HEK-293T (ECACC 12022001), 293-Hektor (ECACC 05030204), 293 N3S (ECACC 92052131), 293 GTP-AC-free (ECACC 05011003), 293TGPRT+R1 (ECACC 04072121), 293TGPRT+R1-A (ECACC 04072120), PC12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), CHO-KI (ECACC 85051005), Vero (ATCC CCL 81), Caco-2 (ATCC HTB 37), K562 (ATCC CCL 243), Jurkat (ATCC TIB-152), Per.C6, 1-Huvec (ATCC Human Primary PCS 100-010, Mouse CRL 2514, CRL 2515, CRL 2516), HuH-7D12 (ECA(CC 01042712), 293 (ATCC CRL 10852), A549 (ATCC CCL 185), IMR-90 (ATCC CCL 186), MCF-7 (ATC HTB-22), U-2 OS (ATCC HTB-96), and T84 (ATCC CCL 248), any established cell line (polarized or nonpolarized), any ATCC and any ECACCcell line.

51. The method of any one of claims 27-50, wherein the gene of interest is a human sensory gene, a taste gene, or an odorant receptor gene.

52. The method of claim 51, wherein the human sensory gene, taste gene, or odorant receptor gene is selected form the group consisting of bitter, salt, sour, umami, hot, pepper, cool, mint, fat, fatty acid, kokumi, mouth feel, touch, touch sensation, mechano sensation, pressure sensing and tingle receptor genes.

53. The method of any one of claims 27-50, wherein the gene of interest is selected from the group consisting of a DNA virus gene, an RNA virus gene, a retrovirus gene, an adeno-associated virus gene, an adenovirus, a lentivirus gene, a herpes virus gene, a HIV gene and a coronavirus gene.

54. The method of any one of claims 27-50, wherein the gene of interest is selected from the group consisting of a SARS gene, a MERS gene, and a SARS-CoV-2 gene.

55. The method of any one of claims 27-50, wherein the gene of interest a viral gene used to produce viral particles for use in gene or cell therapy.

56. The method of any one of claims 27-50, wherein the gene of interest is selected from the group consisting of a gene encoding a cellular protein, a gene encoding a membrane protein, a gene encoding a cytosolic protein, a gene encoding a nuclear protein, a gene encoding a multi-subunit protein, and a gene encoding a secreted protein.

57. The method of any one of claims 27-50, wherein the gene of interest is a drug target or a gene encoding a biologic.

58. The method of any one of claims 27-50, wherein the gene of interest is selected from the group consisting of a gene involved in unfolded protein response, a gene involved in cell viability, a gene involved in protein production, a gene involved in protein folding, a gene involved in protein assembly, a gene involved in protein modification, a gene involved in glycosylation, a gene involved in proteolysis, a gene involved in secretion, a gene involved in cell membrane integration, and a gene involved in cell surface presentation.

59. The method of any one of claims 27-50, wherein the gene of interest is selected from the group consisting of ATF, ATF6, ATF6-Alpha, ATF6A, ATF6B, IRE1 alpha, IRE1α, IRE1 beta, IRE1P, PERCDC, ATF4, YY1, NF-YA, NF-YB, NF-YC, XBP1, XBP2, EDEM1, EDEM2, NRF2, HERP, XIAP, GADD34, PPIA, PPIB, PPIG, DNAJC3, DNAJC6, PRDM1, BLIMP-1, CRT, CALR, CNX, PDIA3, ERp57, HSPA5, BiP, SIL1, BAP, DNAJB11, ERdj3, CaBP1, HSP90B1, GRP-94, PDIA4, ERp72, cyclophilin B, SDF2, SDF2L1, ERO1A, ERO1B, ERAD pathway gene, MAN1B1, mannosidase 1, SYN1, HRD1, STC1, STC2, SERCA1, SERCA2, RPGR, COD1, ISYNA1, INO1, SREBP1, SREBP2, SREBP1DC, SREBP2DC, PC, PYC, Sec61A1, Sec61A2, Sec61B, Sec61G, BCL2, BCL2L1, Bcl-2sp, Bcl-XL, BCL2L11, Bim, XRCC6, Ku70, VDAC2, BCAP31, BAP31, YWHAE, and 14-3-3.

60. A method for preparing a cell comprising a sequence of interest, the method comprising:
(a) incubating the expression vector of any one of claims 1-26 comprising the sequence of interest with a cell;
(b) selecting a cell comprising an integrated expression vector.

61. The method of claim 60, wherein antibiotic resistance is used to select cells in step (b).

62. A cell comprising any one of the expression vectors according to claims 1-61.

63. A cell prepared by the method according to any one of claims 27-59.

64. The cell of claim 62 or claim 63, wherein the cell is a mammalian cell.

65. The cell of claim 64, wherein the mammalian cell is selected from the group consisting of HEK293T and CHO cells.

66. A composition comprising the cell of any one of claims 62-65.

67. A kit comprising an expression vector according to claims 1-26.

68. The kit of claim 67, further comprising a cell.

69. The kit of claim 68, wherein the cell is selected from the group consisting of a NS0, CHO, COS, Perc6, HEK-293, HEK 293T, HUVECs, 3T3 and a HeLa cell.

70. The kit any one of claims 67-69, further comprising a signaling probe.

71. The kit of claim 70, wherein the signaling probe comprises a fluorophore and a quencher.

72. The kit of claim 70 or 71, wherein the signaling probe comprises the nucleotide sequence set forth in SEQ ID NO: 21, 22 or 23.

73. The kit of any one of claims 66-71, further comprising a transfection agent.

74. The kit of claim 73, wherein the transfection agent is selected from Lipofectamine and jetPEI®.
